SEQUENCE LISTING (111 sequences)

SEQ ID NO: 1 - DNA, 20 bp, synthetic construct
  source 1..20: mol_type = other DNA
aattnnnagcgctnnnaatt

SEQ ID NO: 2 - DNA, 21 bp, synthetic construct
  source 1..21: mol_type = other DNA
catccagtttactttgcaggg

SEQ ID NO: 3 - DNA, 25 bp, synthetic construct
  source 1..25: mol_type = other DNA
gataacaaactagcaacaccagaac

SEQ ID NO: 4 - DNA, 26 bp, synthetic construct
  source 1..26: mol_type = other DNA
aaacgcaaagagggaactaaacactt

SEQ ID NO: 5 - DNA, 49 bp, synthetic construct
  source 1..49: mol_type = other DNA
aacttttcggatcaagctatgaaaaacgcaaagagggaactaaacactt

SEQ ID NO: 6 - DNA, 49 bp, synthetic construct
  source 1..49: mol_type = other DNA
ttagttcctcgttttctctcgttaaaagaagaagaaacgagaaactaaa

SEQ ID NO: 7 - DNA, 49 bp, synthetic construct
  source 1..49: mol_type = other DNA
aagtgtttagttccctctttgcgtttttcatagcttgatccgaaaagtt

SEQ ID NO: 8 - DNA, 49 bp, synthetic construct
  source 1..49: mol_type = other DNA
tttagtttctcgtttcttcttcttttaacgagagaaaacgaggaactaa

SEQ ID NO: 9 - DNA, 23 bp, synthetic construct
  source 1..23: mol_type = other DNA
cgacgtccaacatatggcaccgg

SEQ ID NO: 10 - DNA, 24 bp, synthetic construct
  source 1..24: mol_type = other DNA
ggaggctttcgtcttttgacagct

SEQ ID NO: 11 - DNA, 23 bp, synthetic construct
  source 1..23: mol_type = other DNA
ctgagtaggacaaatccgccgcc

SEQ ID NO: 12 - DNA, 24 bp, synthetic construct
  source 1..24: mol_type = other DNA
ctagacctagggtacgggttttgc

SEQ ID NO: 13 - DNA, 53 bp, synthetic construct
  source 1..53: mol_type = other DNA
atgccaacacaattaacatctcaatcaaggtaaatgctttttgctttttttgc

SEQ ID NO: 14 - DNA, 20 bp, synthetic construct
  source 1..20: mol_type = other DNA
tggcaattccgacgtccaac

SEQ ID NO: 15 - DNA, 31 bp, synthetic construct
  source 1..31: mol_type = other DNA
ggaggctttcgtcttttgacagctagctcag

SEQ ID NO: 16 - DNA, 51 bp, synthetic construct
  source 1..51: mol_type = other DNA
agcgagtttttatttcgtttatttcaattaaggtaactaaaaaactccttt

SEQ ID NO: 17 - DNA, 23 bp, synthetic construct
  source 1..23: mol_type = other DNA
acgctctcctgagtaggacaaat

SEQ ID NO: 18 - DNA, 26 bp, synthetic construct
  source 1..26: mol_type = other DNA
ctagacctagggtacgggttttgctg

SEQ ID NO: 19 - DNA, 62 bp, synthetic construct
  source 1..62: mol_type = other DNA
gtttgtaaaggagactgataatggcatgtacaactatactcgtcggtaaaaaggcatcttat

SEQ ID NO: 20 - DNA, 31 bp, synthetic construct
  source 1..31: mol_type = other DNA
aattccgacgtccaacatatggcaccgggtt

SEQ ID NO: 21 - DNA, 22 bp, synthetic construct
  source 1..22: mol_type = other DNA
ttaacggaggctttctcttttg

SEQ ID NO: 22 - DNA, 65 bp, synthetic construct
  source 1..65: mol_type = other DNA
atggataaaaaaatacagcgtttttcatgtacaactatactagttgtagtgcctaaataatgctt

SEQ ID NO: 23 - DNA, 32 bp, synthetic construct
  source 1..32: mol_type = other DNA
ctctcctgagtaggacaaatccgccgcctttt

SEQ ID NO: 24 - DNA, 24 bp, synthetic construct
  source 1..24: mol_type = other DNA
taactagacctagggtacgggttt

SEQ ID NO: 25 - DNA, 61 bp, synthetic construct
  source 1..61: mol_type = other DNA
gagcgccggatcagggagtggacggcctgggagcgctacacgctgtggctgcggtcggtgc

SEQ ID NO: 26 - DNA, 25 bp, synthetic construct
  source 1..25: mol_type = other DNA
tgcagggcttcccaaccttaccaga

SEQ ID NO: 27 - DNA, 26 bp, synthetic construct
  source 1..26: mol_type = other DNA
ttggaggctttcgtcttttgacagct

SEQ ID NO: 28 - DNA, 60 bp, synthetic construct
  source 1..60: mol_type = other DNA
ccctaatacgcaagtcgataactctcctgggagcgttgacaacttgcgcaccctgatctg

SEQ ID NO: 29 - DNA, 25 bp, synthetic construct
  source 1..25: mol_type = other DNA
tcctgagtaggacaaatccgccgcc

SEQ ID NO: 30 - DNA, 25 bp, synthetic construct
  source 1..25: mol_type = other DNA
ctagacctagggtacgggttttgct

SEQ ID NO: 31 - DNA, 65 bp, synthetic construct
  source 1..65: mol_type = other DNA
caatcatcagataactatggcggcacgtgcattaaccacggttgtatcccgtctaaagtactcgt

SEQ ID NO: 32 - DNA, 33 bp, synthetic construct
  source 1..33: mol_type = other DNA
gcaattccgacgtccaacatatggcaccgggtt

SEQ ID NO: 33 - DNA, 30 bp, synthetic construct
  source 1..30: mol_type = other DNA
ttaacggaggctttcgtcttttgacagcta

SEQ ID NO: 34 - DNA, 64 bp, synthetic construct
  source 1..64: mol_type = other DNA
ttaataaactatggaagtatgtacagtcttgcaatgttgagtgaacaaacttccataataaaat

SEQ ID NO: 35 - DNA, 30 bp, synthetic construct
  source 1..30: mol_type = other DNA
taactagacctagggtacgggttttgctgc

SEQ ID NO: 36 - DNA, 66 bp, synthetic construct
  source 1..66: mol_type = other DNA
gttcgtggtaactatgggtggtacaggtgccacattagttgtaccatttatgtttatgtggttaac

SEQ ID NO: 37 - DNA, 31 bp, synthetic construct
  source 1..31: mol_type = other DNA
ggcaattccgacgtccaacatatggcaccgg

SEQ ID NO: 38 - DNA, 33 bp, synthetic construct
  source 1..33: mol_type = other DNA
ggaggctttcgtcttttgacagctagctcagtc

SEQ ID NO: 39 - DNA, 65 bp, synthetic construct
  source 1..65: mol_type = other DNA
tttttgtatgttagttgtgtcactgggtagacctaaatagtgacacaactgctattaaaatttaa

SEQ ID NO: 40 - DNA, 30 bp, synthetic construct
  source 1..30: mol_type = other DNA
cgctctcctgagtaggacaaatccgccgcc

SEQ ID NO: 41 - DNA, 33 bp, synthetic construct
  source 1..33: mol_type = other DNA
ctagacctagggtacgggttttgctgcccgcaa

SEQ ID NO: 42 - DNA, 49 bp, synthetic construct
  source 1..49: mol_type = other DNA
cggccggcttgtcgacgacggcggtctccgtcgtcaggatcatccgggc

SEQ ID NO: 43 - DNA, 23 bp, synthetic construct
  source 1..23: mol_type = other DNA
ggcttcccaaccttaccagaggg

SEQ ID NO: 44 - DNA, 58 bp, synthetic construct
  source 1..58: mol_type = other DNA
gtcgtggtttgtctggtcaaccaccgcggtctcagtggtgtacggtacaaaccccgac

SEQ ID NO: 45 - DNA, 28 bp, synthetic construct
  source 1..28: mol_type = other DNA
ctctcctgagtaggacaaatccgccgcc

SEQ ID NO: 46 - DNA, 28 bp, synthetic construct
  source 1..28: mol_type = other DNA
ctgttctggtgttgctagtttgttatca

SEQ ID NO: 47 - DNA, 59 bp, synthetic construct
  source 1..59: mol_type = other DNA
ggacggcgcagaaggggagtagctcttcgccggaccgtcgacatactgctcagctcgtc

SEQ ID NO: 48 - DNA, 26 bp, synthetic construct
  source 1..26: mol_type = other DNA
cgacgtccaacatatggcaccgggtt

SEQ ID NO: 49 - DNA, 28 bp, synthetic construct
  source 1..28: mol_type = other DNA
ttaacggaggctttcgtcttttgacagc

SEQ ID NO: 50 - DNA, 63 bp, synthetic construct
  source 1..63: mol_type = other DNA
gctcatgtatgtgtctacgcgagattctcgcccgagaacttctgcaaggcactgctcttggct

SEQ ID NO: 51 - DNA, 27 bp, synthetic construct
  source 1..27: mol_type = other DNA
ctgagtaggacaaatccgccgcctttt

SEQ ID NO: 52 - DNA, 31 bp, synthetic construct
  source 1..31: mol_type = other DNA
taactagacctagggtacgggttttgctgcc

SEQ ID NO: 53 - DNA, 16 bp, synthetic construct
  source 1..16: mol_type = other DNA
ttttgagcgctcaaaa

SEQ ID NO: 54 - DNA, 49 bp, synthetic construct
  source 1..49: mol_type = other DNA
ttagttcctcgttttctctcgttaattttgagcgctcaaaaaaactaaa

SEQ ID NO: 55 - DNA, 49 bp, synthetic construct
  source 1..49: mol_type = other DNA
ttttgagcgctcaaaatctcgttaaaagaagaagaaacgagaaactaaa

SEQ ID NO: 56 - DNA, 49 bp, synthetic construct
  source 1..49: mol_type = other DNA
ttagttttttgagcgctcaaaataaaagaagaagaaacgagaaactaaa

SEQ ID NO: 57 - DNA, 49 bp, synthetic construct
  source 1..49: mol_type = other DNA
ttagttcctttttgagcgctcaaaaaagaagaagaaacgagaaactaaa

SEQ ID NO: 58 - DNA, 49 bp, synthetic construct
  source 1..49: mol_type = other DNA
ttagttcctcttttgagcgctcaaaaagaagaagaaacgagaaactaaa

SEQ ID NO: 59 - DNA, 49 bp, synthetic construct
  source 1..49: mol_type = other DNA
ttagttcctcgttttctctcgttaaaattttgagcgctcaaaaactaaa

SEQ ID NO: 60 - DNA, 49 bp, synthetic construct
  source 1..49: mol_type = other DNA
ttagttcctcgttttctctcgttaaaagttttgagcgctcaaaactaaa

SEQ ID NO: 61 - protein, 19 aa, synthetic construct
ATTXYZAGCGGATAACAAT

SEQ ID NO: 62 - protein, 21 aa, synthetic construct
AEYAGVSADTVSRVVNQASHV

SEQ ID NO: 63 - protein, 21 aa, synthetic construct
AATTXYZAGCGGATAACAATT

SEQ ID NO: 64 - DNA, 12 bp, synthetic construct
  source 1..12: mol_type = other DNA
aaagaggagaaa

SEQ ID NO: 65 - DNA, 49 bp, synthetic construct
  source 1..49: mol_type = other DNA
ttagttcctcgttttctctcgttaattcttagcgctaagaaaaactaaa

SEQ ID NO: 66 - DNA, 49 bp, synthetic construct
  source 1..49: mol_type = other DNA
ttagttcctcgttttctctcgttaattgacagcgctgtcaaaaactaaa

SEQ ID NO: 67 - DNA, 49 bp, synthetic construct
  source 1..49: mol_type = other DNA
ttagttcctcgttttctctcgttaattaggagcgctcctaaaaactaaa

SEQ ID NO: 68 - DNA, 49 bp, synthetic construct
  source 1..49: mol_type = other DNA
ttagttcctcgttttctctcgttaattttaagcgcttaaaaaaactaaa

SEQ ID NO: 69 - DNA, 49 bp, synthetic construct
  source 1..49: mol_type = other DNA
ttagttcctcgttttctctcgttaattgtgagcgctcacaaaaactaaa

SEQ ID NO: 70 - DNA, 19 bp, synthetic construct
  source 1..19: mol_type = other DNA
attgtgagcgctcacaatt

SEQ ID NO: 71 - protein, 21 aa, synthetic construct
AEYAGVSYQTVSRVVNQASHV

SEQ ID NO: 72 - DNA, 19 bp, synthetic construct
  source 1..19: mol_type = other DNA
attgacagcgctgtcaatt

SEQ ID NO: 73 - protein, 21 aa, synthetic construct
AEYAGVSGKTVSRVVNQASHV

SEQ ID NO: 74 - DNA, 19 bp, synthetic construct
  source 1..19: mol_type = other DNA
attttaagcgcttaaaatt

SEQ ID NO: 75 - protein, 21 aa, synthetic construct
AEYAGVSTATVSNVVNQASHV

SEQ ID NO: 76 - DNA, 19 bp, synthetic construct
  source 1..19: mol_type = other DNA
attttgagcgctcaaaatt

SEQ ID NO: 77 - protein, 21 aa, synthetic construct
AEYAGVSHQTVSNVVNQASHV

SEQ ID NO: 78 - DNA, 19 bp, synthetic construct
  source 1..19: mol_type = other DNA
attcttagcgctaagaatt

SEQ ID NO: 79 - protein, 21 aa, synthetic construct
AEYAGVSHTTVSKVVNQASHV

SEQ ID NO: 80 - DNA, 19 bp, synthetic construct
  source 1..19: mol_type = other DNA
attaggagcgctcctaatt

SEQ ID NO: 81 - protein, 21 aa, synthetic construct
AEYAGVSKSTVSLVVNQASHV

SEQ ID NO: 82 - DNA, 63 bp, synthetic construct
  source 1..63: mol_type = other DNA
gctcatgtatgtgtctacgcgagattctcgccttgtgagcgctcacaagcactgctcttggct

SEQ ID NO: 83 - DNA, 60 bp, synthetic construct
  source 1..60: mol_type = other DNA
ccctaatacgcaagtcgataactctcctgggagcgttttgagcgctcaaaacctgatctg

SEQ ID NO: 84 - DNA, 65 bp, synthetic construct
  source 1..65: mol_type = other DNA
tttttgtatgttagttgtgtcactgggtagacttgtgagcgctcacaactgctattaaaatttaa

SEQ ID NO: 85 - DNA, 58 bp, synthetic construct
  source 1..58: mol_type = other DNA
gtcgtggtttgtctggtcaaccaccgcggtttttgagcgctcaaaaacaaaccccgac

SEQ ID NO: 86 - DNA, 51 bp, synthetic construct
  source 1..51: mol_type = other DNA
agcgagtttttatttcgtttatttcttttgagcgctcaaaaaaactccttt

SEQ ID NO: 87 - DNA, 62 bp, synthetic construct
  source 1..62: mol_type = other DNA
gtttgtaaaggagactgataatggcatgtacaactatactttgtgagcgctcacaatcttat

SEQ ID NO: 88 - DNA, 65 bp, synthetic construct
  source 1..65: mol_type = other DNA
atggataaaaaaatacagcgtttttcatgtacaactatactttgtgagcgctcacaataatgctt

SEQ ID NO: 89 - DNA, 64 bp, synthetic construct
  source 1..64: mol_type = other DNA
ttaataaactatggaagtatgtacagtcttgcaattgtgagcgctcacaattccataataaaat

SEQ ID NO: 90 - DNA, 64 bp, synthetic construct
  source 1..64: mol_type = other DNA
ttaataaactatggaattgtgagcgctcacaaaatgttgagtgaacaaacttccataataaaat

SEQ ID NO: 91 - protein, 21 aa, synthetic construct
AEYAGVSFKTVSRVVNQASHV

SEQ ID NO: 92 - DNA, 51 bp, synthetic construct
  source 1..51: mol_type = other DNA
agttttgagcgctcaaaattatttcaattaaggtaactaaaaaactccttt

SEQ ID NO: 93 - DNA, 51 bp, synthetic construct
  source 1..51: mol_type = other DNA
agcgagtttttgagcgctcaaaatcaattaaggtaactaaaaaactccttt

SEQ ID NO: 94 - DNA, 51 bp, synthetic construct
  source 1..51: mol_type = other DNA
agcgagtttttatttcgtttatttcaattttgagcgctcaaaaactccttt

SEQ ID NO: 95 - DNA, 65 bp, synthetic construct
  source 1..65: mol_type = other DNA
atggataaaaaaatacagcgtttttcatgtacaactatactattttgagcgctcaaaaaatgctt

SEQ ID NO: 96 - DNA, 65 bp, synthetic construct
  source 1..65: mol_type = other DNA
atggataaaaaaatacagcgtttttcatgtacaactatactagttttgagcgctcaaaaatgctt

SEQ ID NO: 97 - DNA, 65 bp, synthetic construct
  source 1..65: mol_type = other DNA
atggataaaaaaatacagcgtttttcatgtacaactatactagttgttttgagcgctcaaaactt

SEQ ID NO: 98 - DNA, 65 bp, synthetic construct
  source 1..65: mol_type = other DNA
atggataaaaaaatacagcgtttttcatgtacaactatactagttgtttttgagcgctcaaaatt

SEQ ID NO: 99 - DNA, 60 bp, synthetic construct
  source 1..60: mol_type = other DNA
ccctattttgagcgctcaaaactctcctgggagcgttgacaacttgcgcaccctgatctg

SEQ ID NO: 100 - DNA, 65 bp, synthetic construct
  source 1..65: mol_type = other DNA
tttttgtatgttagttgtgtcactgggtagacctaaatagtgttttgagcgctcaaaaaatttaa

SEQ ID NO: 101 - DNA, 69 bp, synthetic construct
  source 1..69: mol_type = other DNA
ttagttcctcgttttctctcgttaatccctatcagtgatagagactaaactccctatcagtgatagaga

SEQ ID NO: 102 - DNA, 1083 bp, synthetic construct
  source 1..1083: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 103 - DNA, 1014 bp, synthetic construct
  source 1..1014: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatcaaagagctgggctacgtgccgaaccgcgcagcccgcaccctggtcacccgacgtaccgacaccgtagccctggtggtgtcggaaaacaaccagaagctcttcgccgaacccttctatgccgggatcgtgctcggcgtgggggttgctctgtccgaacggggattccagttcgtcctggccacgggccgctccgggatagagcatgagcggctgggcggctacctggccggacagcacgtcgacggggtcctcctgctgtcgctccaccgcgacgacccgctgccgcagatgctggacgaggccggggtgccgtacgtctacggcggccgtccgctcggcgtccccgaagaacaggtgtcctatgtcgatatcgacaacatcggcgggggacgccaggccacccagcggctgatcgagaccgggcaccggcggatcgctacgatcgcgggcccgcaggacatggtcgctggtgtggaacgcctccaggggtatcgcgaagcactgctcgccgcggggatggagtacgacgagacgctggtgagctacggtgacttcacctacgacagcggggtggccgcgatgcgggagctgctggatcgggcccccgacgtggacgccgtgttcgcggcctccgacttgatggggctggccgcgctgcgggtgctgcgtgcttcgggacgccgcgtgcccgaggatgtggcggtggtcggctacgacgactcgaccgtagccgagcacgccgaaccgccgatgaccagcgtcaaccagcccaccgagctgatgggccgggagatggcccggctgctcgtggaccggatcaccggggagaccaccgaaccggtgcggctggtgctggagacccatttgatggtgcgggaatccgggtga

SEQ ID NO: 104 - DNA, 999 bp, synthetic construct
  source 1..999: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcacaacaactggcgggcaaagcgtcgcatacaattggcatgttgatcactgccagtaccaatcctttctattcagaactggtgcgtggcgttgaacgcagctgcttcgaacgcggttatagtctcgtcctttgcaataccgaaggcgatgaacagcggatgaatcgcaatctggaaacgctgatgcaaaaacgcgttgatggcttgctgttactgtgcaccgaaacgcatcaaccttcgcgtgaaatcatgcaacgttatccgacagtgcctactgtgatgatggactgggctccgttcgatggcgacagcgatcttattcaggataactcgttgctgggcggagacttagcaacgcaatatctgatcgataaaggtcatacccgtatcgcctgtattaccggcccgctggataaaactccggcgcgcctgcggttggaaggttatcgggcggcgatgaaacgtgcgggtctcaacattcctgatggctatgaagtcactggtgattttgaatttaacggcgggtttgacgctatgcgccaactgctatcacatccgctgcgtcctcaggccgtctttaccggaaatgacgctatggctgttggcgtttaccaggcgttatatcaggcagagttacaggttccgcaggatatcgcggtgattggctatgacgatatcgaactggcaagctttatgacgccaccattaaccactatccaccaaccgaaagatgaactgggggagctggcgattgatgtactcatccatcggataacccagccgacccttcagcaacaacgattacaacttactccgattctgatggaacgcggttcggcttag

SEQ ID NO: 105 - DNA, 1038 bp, synthetic construct
  source 1..1038: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggagtctcttagctatcacccgaacgccaacgcccgtgcgctggcgcagcagaccactgaaacggtcggtctggtcgttggtgatgtttccgatccgtttttcggtgcaatggtgaaagcggtcgaacaggtggcttatcacaccggtaattttttattgattggcaacggttaccacaacgaacaaaaagagcgtcaggccattgagcaactgatccgccatcgctgtgctgcgttggtcgtccatgccaaaatgatcccggatgctgatttagcctcattaatgaaacaaatgcccggtatggtgctgatcaaccgtatcctgcctggctttgaaaaccgttgtattgctctggacgatcgttacggtgcctggctggcaacgcgtcatttaattcagcaaggtcatacccgcattggttatctgtgctctaaccactctatttctgacgccgaagatcgtctgcaagggtattacgatgcccttgctgaaagtggtattgcggccaatgaccggctggtgacatttggcgaaccagacgaaagcggcggcgaacaggcaatgaccgagcttttgggacgaggaagaaatttcactgcggtagcctgttataacgattcaatggcggcgggtgcgatgggcgttctcaatgataatggtattgatgtaccgggtgagatttcgttaattggctttgatgatgtgctggtgtcacgctatgtgcgtccgcgcctgaccaccgtgcgttacccaatcgtgacgatggcgacccaggctgccgaactggctttggcgctggcggataatcgccctctcccggaaatcactaatgtctttagtccgacgctggtacgtcgtcattcagtgtcaactccgtcgctggaggcaagtcatcatgcaaccagcgactaa

SEQ ID NO: 106 - DNA, 1005 bp, synthetic construct
  source 1..1005: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcacaacaactggcgggcaaaagtagccgttctattggtcttgtgatccccgatctggagaacaccagctatacccgcatcgctaactatcttgaacgccaggcgcggcaacggggttatcaactgctgattgcctgctcagaagatcagccagacaacgaaatgcggtgcattgagcaccttttacagcgtcaggttgatgccattattgtttcgacgtcgttgcctcctgagcatcctttttatcaacgctgggctaacgacccgttcccgattgtcgcgctggaccgcgccctcgatcgtgaacacttcaccagcgtggttggtgccgatcaggatgatgccgaaatgctggcggaagagttacgtaagtttcccgccgagacggtgctttatcttggtgcgctaccggagctttctgtcagcttcctgcgtgaacaaggtttccgtactgcctggaaagatgatccgcgcgaagtgcatttcctgtatgccaacagctatgagcgggaggcggctgcccagttattcgaaaaatggctggaaacgcatccgatgccgcaggcgctgttcacaacgtcgtttgcgttgttgcaaggagtgatggatgtcacgctgcgtcgcgacggcaaactgccttctgacctggcaattgccacctttggcgataacgaactgctcgacttcttacagtgtccggtgctggcagtggctcaacgtcaccgcgatgtcgcagagcgtgtgctggagattgtcctggcaagcctggacgaaccgcgtaagccaaaacctggtttaacgcgcattaaacgtaatctctatcgccgcggcgtgctcagccgtagctaa

SEQ ID NO: 107 - DNA, 999 bp, synthetic construct
  source 1..999: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcacaacaactggcgggcaaagcgtcgcataccattggcatgttgatcactgccagtaccaatcctttctattcagaactggtgcgtggcgttgaacgcagctgcttcgaacgcggttatagtctcgatctttgcaataccgaaggcgatgaacagcggatgaatcgcaatctggaaacgctgatgcaaaaacgcgttgatggcttgctgttactgtgcaccgaaacgcatcaaccttcgcgtgaaatcatgcaacgttatccgacagtgcctactgtgatgatggactgggctccgttcgatggcgacagcgatcttattcaggataactcgttgctgggcggagacttagcaacgcaatatctgatcgataaaggtcatacccgtatcgcctgtattaccggcccgctggataaaactccggcgcgcctgcggttggaaggttatcgggcggcgatgaaacgtgcgggtctcaacattcctgatggctatgaagtcactggtgattttgaatttaacggcgggtttgacgctatgcgccaactgctatcacatccgctgcgtcctcaggccgtctttaccggaaatgacgctatggctgttggcgtttaccaggcgttatatcaggcagagttacaggttccgcaggatatcgcggtgattggctatgacgatatcgaactggcaagctttatgacgccaccattaaccactatccaccaaccgaaagatgaactgggggagctggcgattgatgtactcatccatcggataacccagccgacccttcagcaacaacgattacaacttactccgattctgatggaacgcggttcggcttag

SEQ ID NO: 108 - DNA, 1005 bp, synthetic construct
  source 1..1005: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcacaacaactggcgggcaaaagttcgcgttctattggccttgtgatccccgatctggagaacaccagctattcccgcttcgctaactatctcgaacgccaggcgcggcaacggggttatcaactgaaaattgcctgctcagaagatcagccagacaacgaaatgcggtgcattgagcaccttttacagcgtcaggttgatgccattattgtttcgacgtcgttgcctcctgagcatcctttttatcaacgctgggctaacgacccgttcccgattgtcgcgctggaccgcgccctcgatcgtgaacacttcaccagcgtggttggtgccgatcaggatgatgccgaaatgctggcggaagagttacgtaagtttcccgccgagacggtgctttatcttggtgcgctaccggagctttctgtcagcttcctgcgtgaacaaggtttccgtactgcctggaaagatgatccgcgcgaagtgcatttcctgtatgccaacagctatgagcgggaggcggctgcccagttattcgaaaaatggctggaaacgcatccgatgccgcaggcgctgttcacaacgtcgtttgcgttgttgcaaggagtgatggatgtcacgctgcgtcgcgacggcaaactgccttctgacctggcaattgccacctttggccagaacgaactgctcgacttcttacagtgtccggtgctggcagtggctcaacgtcaccgcgatgtcgcagagcgtgtgctggagattgtcctggcaagcctggacgaaccgcgtaagccaaaacctggtttaacgcgcattaaacgtaatctctatcgccgcggcgtgctcagccgtagctaa

SEQ ID NO: 109 - DNA, 1047 bp, synthetic construct
  source 1..1047: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctcaattacattcccaaccgcgtggcagcccaactggcgggcaaacagtcggacaccataggcgtggtggtgatggacgttactgatgcgtttttcggcgcgctggtaaaagcggtggatcaggtcgctcagcagcatcagaaatacgtggcaatcggcaatagctatcatgaagcggaaaaagagcgtcacgccattgaggtgttaattcgccagcgttgtaatgcgttgattgttcactcaaaagcattgagtgacgatgaactggcgcaatttatggataacattcccggtatggtgttaatcaaccgcgttgtgccggggtacgcccatcgttgcgtttgtctggataatctcagcggtgcccgaatggcgacgcgcatgttgctgaataacggtcatcaacgtattggttatctttcttccagccacggcattgaagatgacgccatgcgtaaagcaggctggatgagtgcgttgaaagagcaggatattattccgccggaaagctggattggcgctggtacgccggacatgccgggcggtgaggcggcgttggttaaactgctggggcgcaatctacaacttaccgctgtatttgcttataacgacaatatggccgctggcgcactgacagcattaaaagataatggcattgcgattccgttacatctctcaatcatcgggttcgatgatattcccatcgcccgttacaccgacccgcaattaacgaccgtgcgttatcccattgcttcaatggcgaaattagccaccgaactggccttgcagggggcagcaggcaatattgatcctcgtgccagccactgttttatgccgacgttagtgcgtcgccattctgtcgcaacacgccagaatgcggcggcgatcactaactcaacaaatcaggcgatgtaa

SEQ ID NO: 110 - DNA, 1032 bp, synthetic construct
  source 1..1032: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctccactacattcctagcgcggtggcgcgtagcctggcgggcaaacagtcgaagtctatcggtttgctggcgaccagcagcgaagcggcctattttgccgagatcattgaagcagttgaaaaaaattgcttccagaaaggttacaccctgattctgggcaatgcgtggaacaatcttgagaaacagcgggcttatctgtcgatgatggcgcaaaaacgcgtcgatggtctgctggtgatgtgttctgagtacccagagccgttgctggcgatgctggaagagtatcgccatatcccaatggtggtcatggactggggtgaagcaaaagctgacttcaccgatgcggtcattgataacgcgttcgaaggcggctacatggccgggcgttatctgattgaacgcggtcaccgcgaaatcggcgtcatccccggcccgctggaacgtaacaccggcgcaggccgccttgccggttttatgaaggcgatggaagaagcgatgatcaaggtgccggaaagctggattgtgcagggtgactttgaacctgaatccggttatcgcgccatgcagcaaatcctgtcgcagccgcatcgccctactgccgtcttctgtggtggcgatatcatggcaatgggcgcactttgtgctgctgatgaaatgggcctgcgcgtcccgcaggatgtttcgctgatcggttatgataacgtgcgcaacgcgcgctattttacgccggcgctgaccacgatccatcagccaaaagattcgctgggtgaaacagcgttcaacatgctgttggatcgtatcgtcaacaaacgtgaagaaccgcagtctattgaagtgcatccgcgcttgattgaacgccgctccgtggctgacggcccgttccgcgactatcgtcgttaa

SEQ ID NO: 111 - DNA, 624 bp, synthetic construct
  source 1..624: mol_type = other DNA
atgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaa

(19) United States
(12) Patent Application Publication
(10) Pub. No.: US 2025/0043287 A1
(43) Pub. Date: Feb. 6, 2025

(54) SYNTHETIC MEMORY WITH INTERCEPTING RECOMBINASE FUNCTION

(71) Applicant: Georgia Tech Research Corporation, Atlanta, GA (US)

(72) Inventor: Corey J. Wilson, Atlanta, GA (US)

(21) Appl. No.: 18/790,573

(22) Filed: Jul. 31, 2024

Related U.S. Application Data
(60) Provisional application No. 63/516,677, filed on Jul. 31, 2023.

(51) Int. Cl. C12N 15/113; C12N 9/22; C12N 15/10

(52) U.S. Cl. CPC C12N 15/113; C12N 9/22; C12N 15/102

(57) ABSTRACT

Disclosed herein is a synthetic memory system, comprising: a recombinase; a modified transcription factor; and a first nucleic acid comprising a first attachment site, a second attachment site, and a target gene between said first attachment site and said second attachment site; wherein at least one of the first attachment site and the second attachment site comprises a modified DNA operator to which the modified transcription factor can reversibly bind; and wherein, when the modified transcription factor is bound to the modified DNA operator, the recombinase is blocked from binding to the attachment site comprising said modified DNA operator.

### CROSS REFERENCE TO RELATED APPLICATIONS

This application claims the benefit of priority to U.S. Provisional Application No. 63/516,677, filed Jul. 31, 2023, which is incorporated by reference herein in its entirety.

### STATEMENT OF GOVERNMENT SUPPORT

This invention was made with government support under Contract No. 2123855, Contract No. 1934836, Contract No. 1921061, Contract No. 1804639, Contract No. 1844289, and Contract No. 1747439, awarded by the National Science Foundation. The government has certain rights in the invention.

### REFERENCE TO SEQUENCE LISTING

The sequence listing submitted on Jul. 31, 2024, as an .XML file entitled “txt_10034-297US1.txt” created on Jul. 25, 2024, and having a file size of 110,380 bytes is hereby incorporated by reference pursuant to 37 C.F.R. § 1.52 (e)(5).

### BACKGROUND

Intelligent chassis cells have been developed as a synthetic biotic system capable of decision-making and memory operations. Synthetic memory has been demonstrated in myriad ways, e.g., bistable toggle switches, CRISPR-based editing, and recombinase facilitated DNA rearrangements.

In certain systems, the decision-making can employ one or more INPUT(s) mapped to an OUTPUT such that the system can be reset upon the removal of the INPUT(s). Synthetic memory, in contrast, does not reset upon the removal of cognate INPUT(s); that is, memory operations retain changes in the OUTPUT state upon the removal of the cognate INPUT(s).

Serine integrases are a class of enzymes that site-specifically bind and reconfigure sets of DNA elements, e.g., attachment sites attB and attP, to provide DNA deletion or DNA inversion, dependent on the orientation of the set of attachment sites. Serine integrases have been deployed for a broad range of purposes.

There is a benefit to improving synthetic memory.

### SUMMARY

An exemplary interception synthetic memory system and method are disclosed that employs engineered repressors (e.g., CelR, LacI, RbsR, GalR, and FruR), engineered anti-repressors (e.g., anti-LacI, anti-RbsR, anti-FruR, anti-GalS, and PurR), recombinases (e.g., A118, TP901, Bxb1, Int2, Int3, Int5, Int8, and Int12) and cognate attachment sites. The interception synthetic memory can be employed to facilitate: (i) a programmed loss-of-function (LOF) via post-translationally induced deletion, (ii) a programmed gain-of-function (GOF) by way of post-translationally regulated inversion, and (iii) a programmed synthetic interception memory with nested Boolean logical operations.

Interception can provide a significantly faster response than canonical recombinase-based memory and can support multiple synthetic transcription factors in addition to natural transcription factors. Interception synthetic memory can additionally facilitate the development of biology circuits for myriad applications in biological security, living therapeutics, biomanufacturing, and the like.

Examples are provided using the iteration of GOF and LOF defined by Huang et al. [1], among others, which utilizes an inert reporter OUTPUT (e.g., green fluorescent protein) as a proxy for function, though it is contemplated that other program functions may be employed. A study was conducted that demonstrated that interception synthetic memory capacity was expanded via the re-design of the central conserved region of a given set of attachment sites, allowing multiple orthogonal interception synthetic memory events via a single recombinase. The study also illustrated that interception-regulated synthetic memory is significantly faster than previous iterations of recombinase-based memory.

The exemplary synthetic biological memory can be expandable. For example, for 6-orthogonal attachment sites that correspond to A118 and 5 orthogonal synthetic transcription factors, the exemplary synthetic biological memory can have the capacity for a single recombinase to be expanded at least 5-fold. This can support concurrent multiple aligned and anti-aligned attachment site orientations and exceed the current state of the art.

In an aspect, provided is a synthetic memory system, including: a recombinase; a modified transcription factor; and a first nucleic acid including a first attachment site, a second attachment site, and a target gene between said first attachment site and said second attachment site; wherein at least one of the first attachment site and the second attachment site includes a modified DNA operator to which the modified transcription factor can reversibly bind; and wherein, when the modified transcription factor is bound to the modified DNA operator, the recombinase is blocked from binding to the attachment site including said modified DNA operator.

In another aspect, provided is a method of executing a memory operation, the method including: a) providing a synthetic memory system including a recombinase; a modified transcription factor; and a first nucleic acid including a first attachment site, a second attachment site, and a target gene between said first attachment site and said second attachment site; wherein at least one of the first attachment site and the second attachment site includes a modified DNA operator to which the modified transcription factor can reversibly bind; and wherein, when the modified transcription factor is bound to the modified DNA operator, the recombinase is blocked from binding to the attachment site including said modified DNA operator; and b) adding or removing an input; wherein the modified transcription factor responds to the addition or removal of said input by modifying binding of said transcription factor and triggering the memory operation.

### BRIEF DESCRIPTION OF DRAWINGS

FIGS. 1A-1G depict a schematic representation of interception systems. FIG. 1A shows the decision-making rule as bidirectional between STATE 1 and STATE 2. The representative logical operations in decision-making are BUFFER and NOT logical operations. When the INPUT is absent, the OUTPUT is OFF in BUFFER and ON in NOT operation (STATE 1). When the INPUT is present, the OUTPUT is ON in BUFFER and OFF in NOT operation (State 2). Once the INPUT is removed, the OUTPUT state reverts to State 1. FIG. 1B shows the synthetic memory rule as unidirectional between STATE 1 and STATE 2. The representative synthetic memory operations are Gain of Function (GOF) and Loss of Function (LOF). The INPUT and OUTPUT state in synthetic memories are the same as decision-making, however, when the INPUT is removed, the OUTPUT state does not go back to STATE 1. FIGS. 1C-1D show two types of recombination events. When attB and attP are aligned, recombination results in deletion of the DNA element between attB and attP (FIG. 1C). When attB and attP are anti-aligned, recombination results in inversion of the DNA element between attB and attP (FIG. 1D). Note: The icon for the recombinase is given as a monomer. FIG. 1E shows two types of transcription factors used for interception are shown. The blue box shows the repressor mechanism, and the purple box shows the anti-repressor mechanism. The right panel illustrates the regulatory protein template. This system consists of three parts: a dimeric regulatory core domain (RCD or anti-RCD), alternate DNA recognition (ADR), and DNA operator (O^{NNN}). The RCD can be abbreviated as I, G, S, R, E, or F and the superscript+ or A represents the repressor or anti-repressor phenotype, respectively. Each RCD has a cognate inducer shown as a colored hexagon. The ADRs are named via the mutation of amino acid positions 17, 18, and 22. DNA operators are named via nucleotide substitutions at positions 6, 5, and 4 relative to the left half-site of the operator (abbreviated as O^{N6,N5,N4}). Each ADR binds a cognate DNA operator shown color-coded in the bottom-right box. FIG. 1F is a schematic showing the seven steps that must be completed following a type-I memory circuit's induction for recombination to occur. FIG. 1G is a schematic showing the four steps that must be completed following a type-II memory circuit's induction for recombination to occur. Sequences in FIGS. 1A-1G: 5′-AATT N°N5N4 AGCGCT N′N′N′ AATT-3′ (SEQ ID NO: 1).

FIGS. 2A-2B depict illustrations and iconography for deletion and inversion synthetic memory. FIG. 2A shows a granular description of the set if A118 recombinase attachment sites in the aligned (deletion) configuration (left) and the iconography for the two aligned orientations that result in deletion (right). FIG. 2B shows a detailed description of anti-aligned (inversion) attachment sites for the A118 recombinase (top), and below is the iconography for the two anti-aligned orientations that result in inversion. Note: The icon for the recombinase is given as a monomer. Sequences in FIGS. 2A-2B:

(SEQ ID NO: 6)
TTAGTTCCTCGTTTTCTCTCGTTAAAAGAAGAAGAAACGAGAAACTAAA,
(SEQ ID NO: 7)
3′-TTGAAAAGCCTAGTTCGATACTTTTTGCGTTTCTCCCTTGATTTGTG
AA-5′,
(SEQ ID NO: 8)
5′-TTTAGTTTCTCGTTTCTTCTTCTTTTAACGAGAGAAAACGAGGAACT
AA-3′.

FIGS. 3A-3H depict multiple input interception memory. FIG. 3A shows a schematic of the interception of a deletion circuit by two transcription factors (TFs). The regulatory core domains and DNA binding domains vary, and corresponding ligands and DNA operators are used in each case. A118 recombinase is constitutively expressed in all cases, and the variable operators are always placed at the P+1 position. The state diagram presents the relationship between repressor, ligand, operator, and recombinase with each set of ligands added. FIGS. 3B-3F show interception with two repressors. FIG. 3B shows E⁺_{YQR} and I⁺_{YQR} binding to O^{SYM}. FIG. 3C shows E⁺_{YQR} and R⁺_{YQR} binding to O^{SYM}. FIG. 3D shows E⁺_{HON} and I⁺_{HON} binding to Ong. FIG. 3E shows E⁺_{KSL}, and R⁺_{KSI}, binding to O^{agg}. FIG. 3F shows E⁺_{KSI}, and I⁺_{KSI}, binding to O^{agg}. FIG. 3G shows interception with two anti-repressors, R^{A}_{HQN} and I^{A}_{HQN} binding to O^{ttg}. The inset schematic shows the relationship between anti-repressors, ligand, and operator. FIG. 3H shows interception with one repressor, E⁺_{HQN}, and one anti-repressor, I^{A}_{HQN}, binding to O^{ttg}. Source data are provided as a Source Data file. Data in FIGS. 3B-3H represent the average of n=6 biological replicates. Error bars correspond to the SEM of these measurements.

FIGS. 4A-4D depict engineering interception att orthogonality and memory kinetics. FIG. 4A shows a recombination matrix for A118 attachment-site pairs with matched (along the diagonal) and mismatched (off-diagonal) central dinucleotides. Rows correspond to attB sites having the central dinucleotide listed at left, and columns correspond to attP sites having the central dinucleotide listed across the top. Each box displays GFP output for a deletion circuit having the corresponding attachment sites in the presence of constitutive A118 expression, as shown at the bottom. The relative expression level of GFP is shown inside each box. Controls for attachment site pairs with matched central dinucleotides and no A118 expression are shown to the right of the matrix. Scale bar reference for GFP output is scaled to each row's maximum GFP value. In FIG. 4B, at left, is shown a genetic schematic for a two-channel deletion circuit containing two fluorescent outputs (mKate and GFP). Below assay data is shown for this two-output circuit co-transformed with a constitutive I⁺_{KSL} and E⁺_{HON} expression plasmid and a constitutive A118-expression plasmid under the four different INPUT conditions shown at the bottom. FIG. 4C shows kinetic assay data over three days for the type-I memory circuit shown in FIG. 1F. On the plot at left, boxed in gray, is control data for cells containing only the GFP reporter plasmid (Reporter Control). The center three bars represent the circuit with no inducer (cellobiose) added and correspond to A118 transcription being repressed over three days. The three bars at right represent the circuit with the inducer added and correspond to A118 transcription being on for three days. In FIG. 4D, at right, kinetic assay data over three days is shown for type-II (interception) memory shown below, with the same inducer conditions described in FIG. 4C. Note: The icon for the recombinase is given as a monomer. Source data are provided as a Source Data file. Data in FIGS. 4A-4D represent the average of n=6 biological replicates. Error bars correspond to the SEM of these measurements.

FIGS. 5A-5C depict the qualitative genotype of 8 recombinases paired with cognate interception deletion circuits. FIG. 5A shows a schematic of an interception deletion circuit in which a constitutive promoter, ribozyme, RBS, and fluorescent protein GFP are nested between an aligned attB and attP pair (STATE 1). A recombinase matched to those att sites catalyzes recombination between attB and attP, resulting in the deletion of GFP expression (STATE 2). An Ong operator is included at position P+1. FIG. 5B shows PCR primers that bind outside the reporter circuit can be used with gel electrophoresis to differentiate between an intact GFP circuit (state 1:1,300 bp) and the recombined circuit (state 2:200 bp). FIG. 5C shows gel electrophoresis of colony PCR of the reporter circuit for cells transformed with only reporter and no recombinase (labeled “S1”) versus cells transformed with reporter and recombinase (shown as “+ recombinase symbol”).

FIGS. 6A-6C depict the gating method used for flow cytometer analysis. FIG. 6A shows cells were first gated by forward scatter (FSC)-area and side scatter (SSC)-area represented as P1 (left). Then the gated cells were further gated again by SSC-area and SSC-height represented as P2 (middle). Then this P2 population was gated by the FITC-height value (right). The population having a higher than 3E3 FITC-H value is considered as GFP ON, and that lower than 3E3 is considered as GFP OFF. FIG. 6B shows the gating method used for GFP and mKate. Similarly, the population higher than 3E3 FITC-H value is considered as GFP ON, and that lower than 3E3 is considered as GFP OFF. The population higher than 2E3 ECH-H value is considered as mKate ON, and that lower than 2E3 is considered as mKate OFF. FIG. 6C shows the 2-D plot of GFP mKate circuit to see how much population is OFF in both inducer states. The gain of the flow cytometer setting is followed: FSC (105), SSC (139), FITC (20), and ECD (1000).

FIGS. 7A-7H depict the definition and analysis of recombinase attachment half-site omission. Each recombinase recognizes and recombines two attachment sites, attB, and attP. Each attachment site contains a central conserved region, shown in bold grey, such that attB and attP must be identical when aligned for deletion or complementary when aligned for inversion1 (also see FIGS. 2A-2B). Attachment site sequences are shown in blue, and central conserved regions are shown in bold grey. FIG. 7A shows symbols corresponding to recombinase half-attachment-site sequence omissions for A118. For example, A118 B1 corresponds to a truncated attachment site lacking the indicated sequence, i.e., AAACGCAAAGAGGGAACTAAACACTT (SEQ ID NO: 4). In other words, the truncated triangle symbol refers schematically to the segment of the attachment site sequence that has been omitted. The DNA sequence on the original reporter construct (pSK001) upstream of attB is now present in place of the half-site B1. At right, data corresponding to the half-site omission experiment described in FIGS. 8A-8L is shown. FIGS. 7B-7H The attachment site sequences and corresponding half-site sequence omissions are shown with the relevant assay data for the recombinases A118, TP901, Int2, Int3, Int5, Int8, Int12, and Bxb1. Source data are provided as a Source Data file. Data in FIGS. 7A-7H represent the average of n=6 biological replicates. Error bars correspond to the SEM of these measurements. Sequences in FIGS. 7A-7H are provided in TABLE 1.

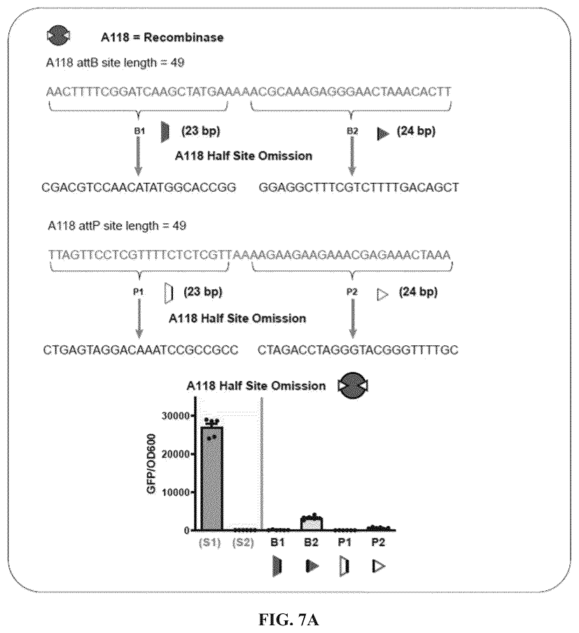

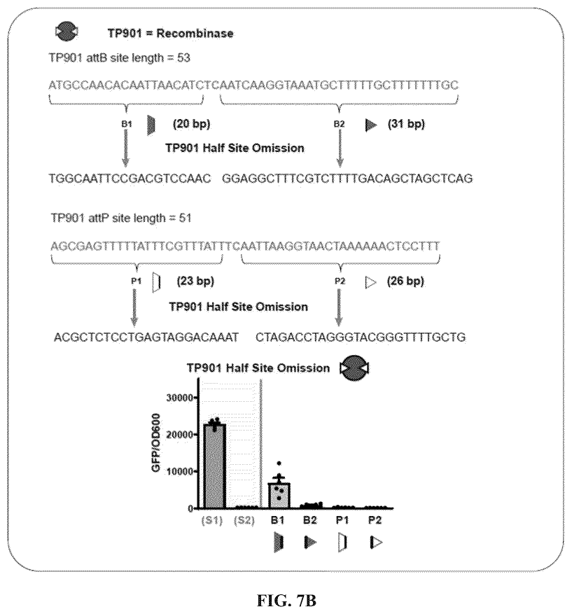

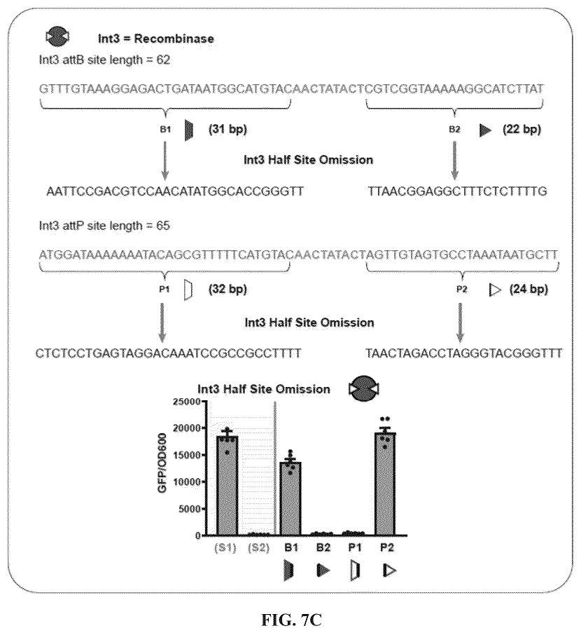

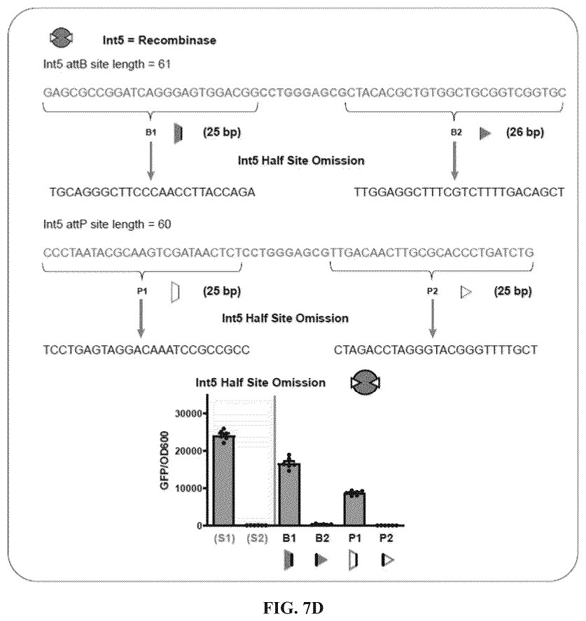

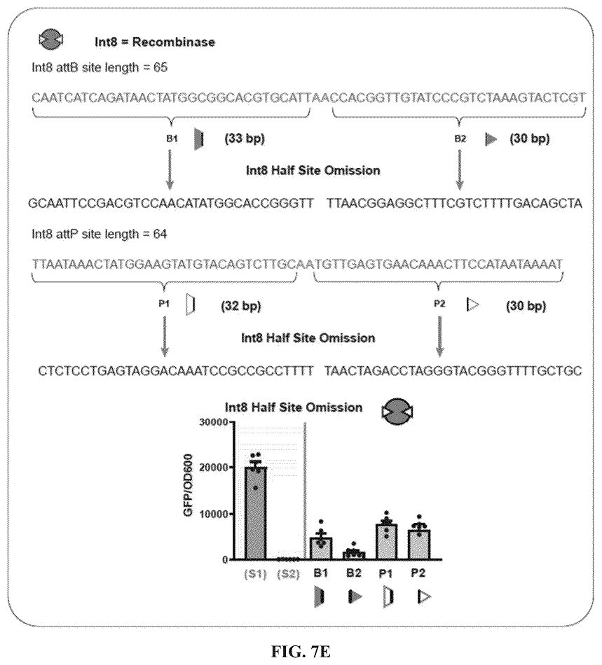

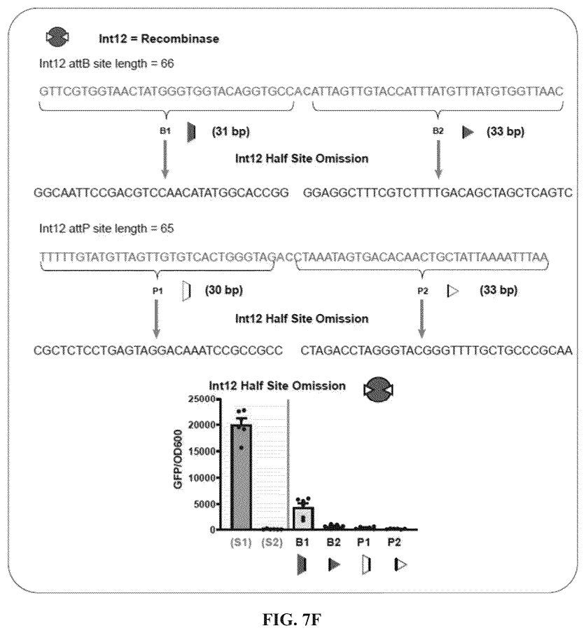

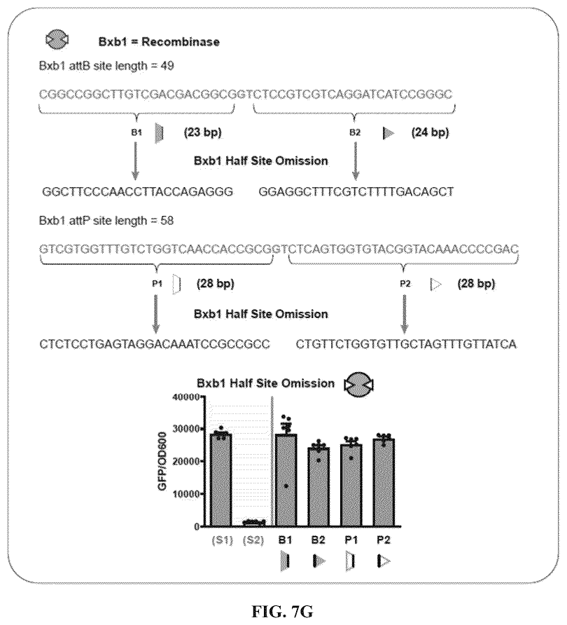

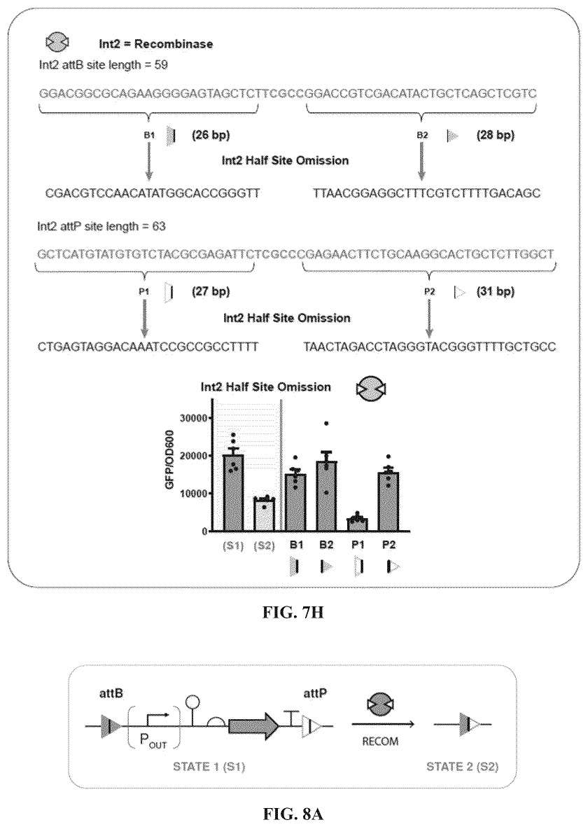

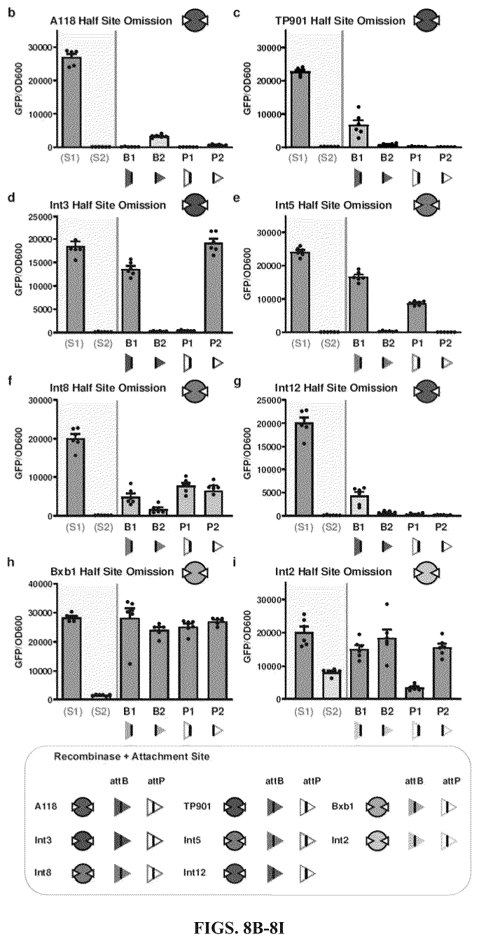

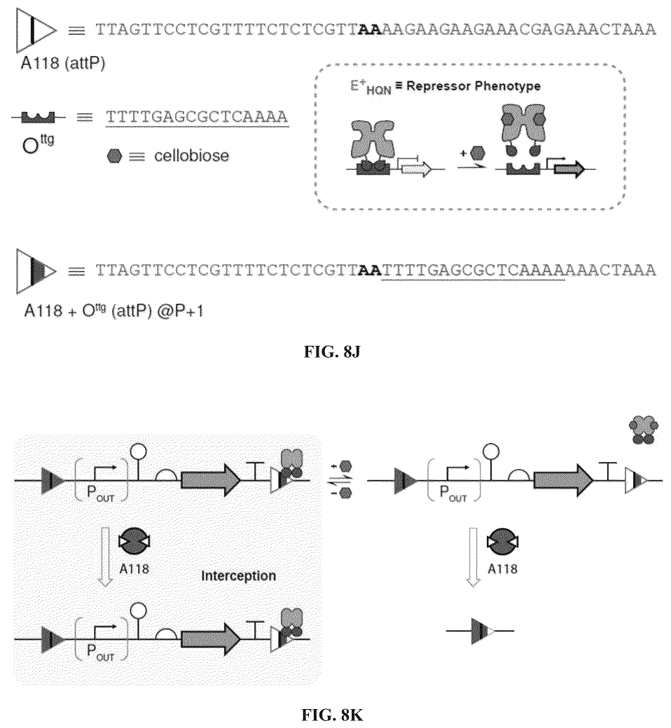

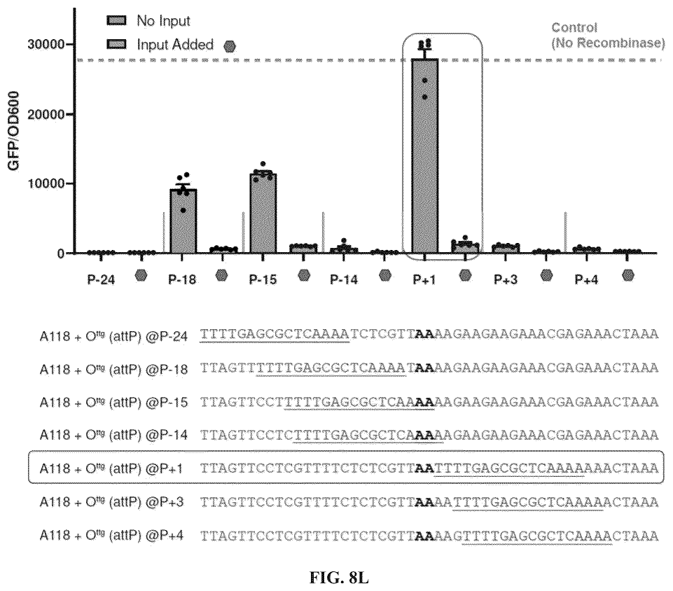

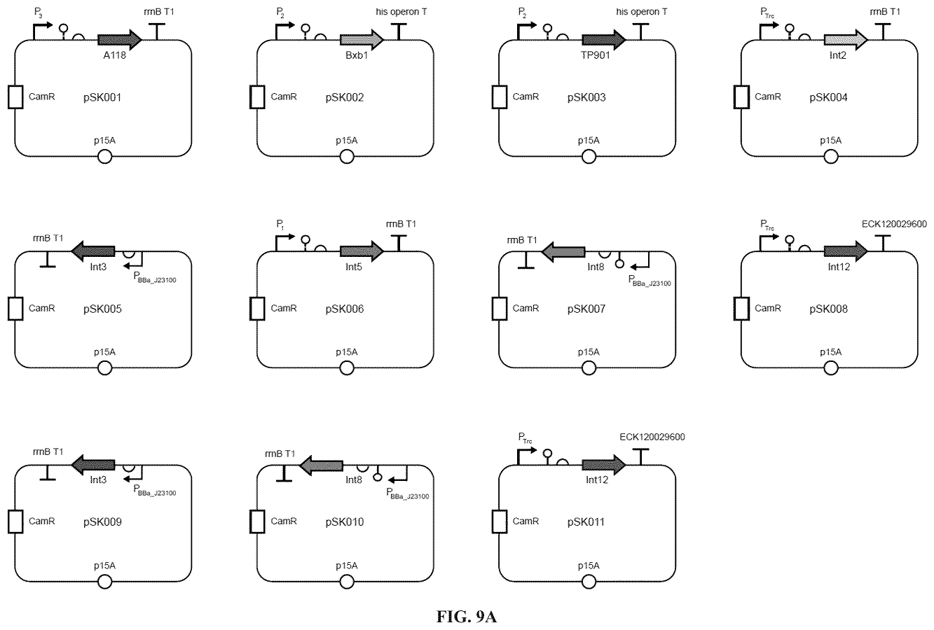

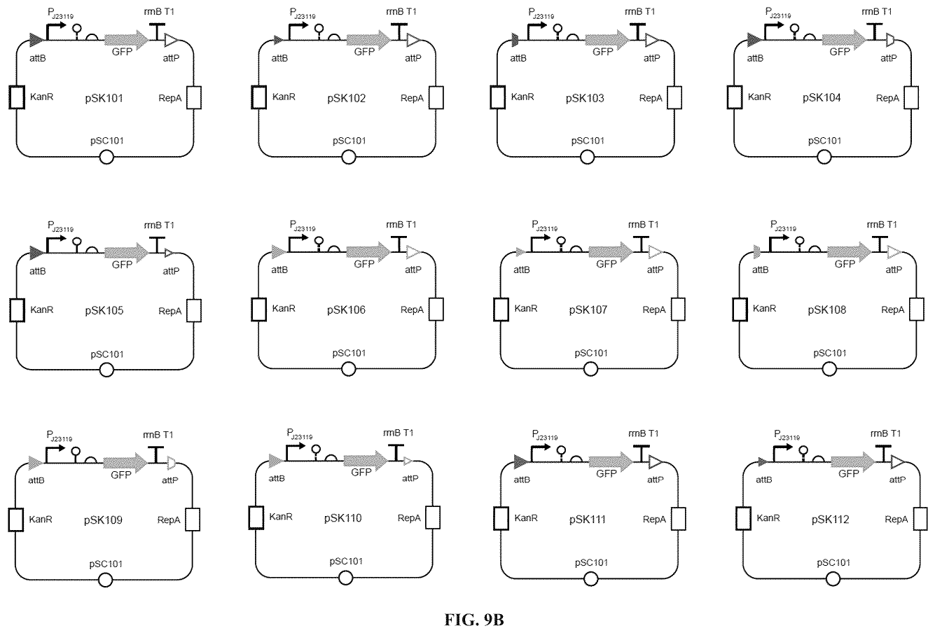

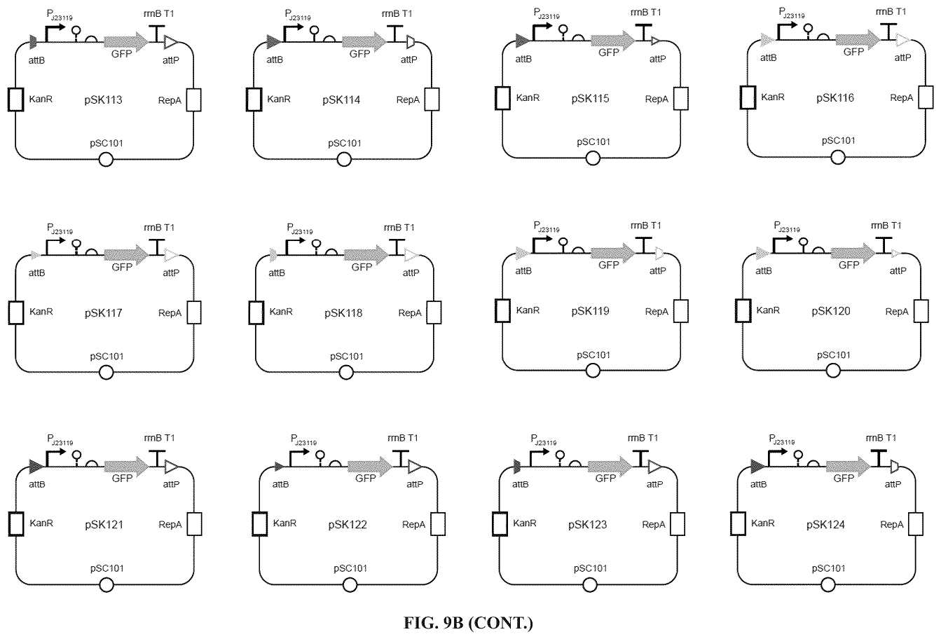

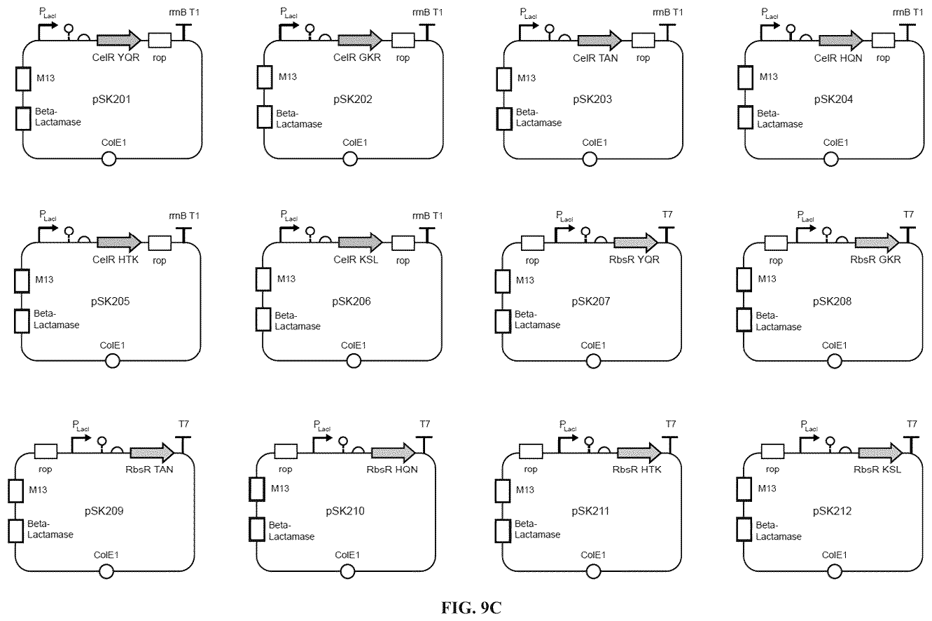

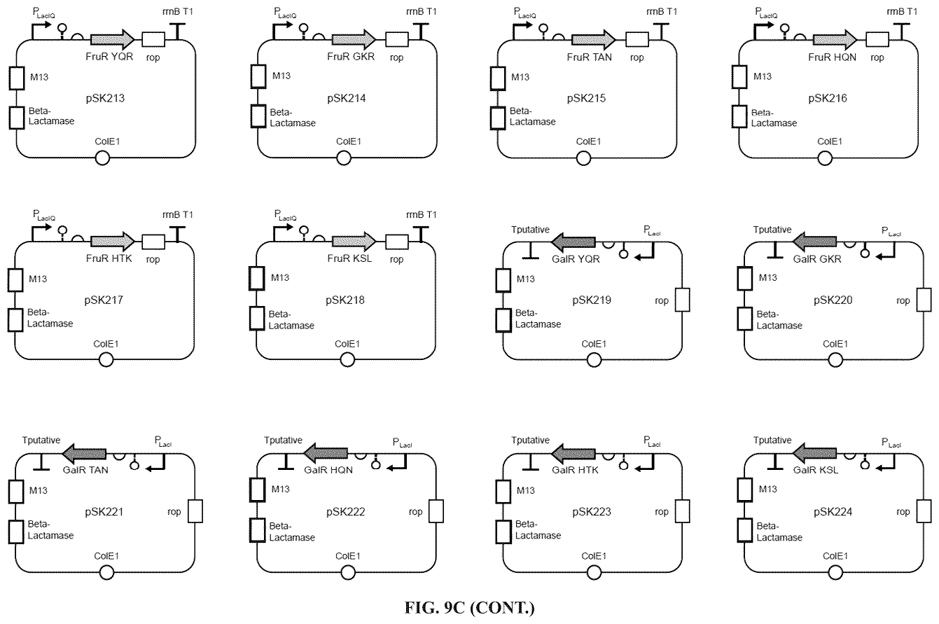

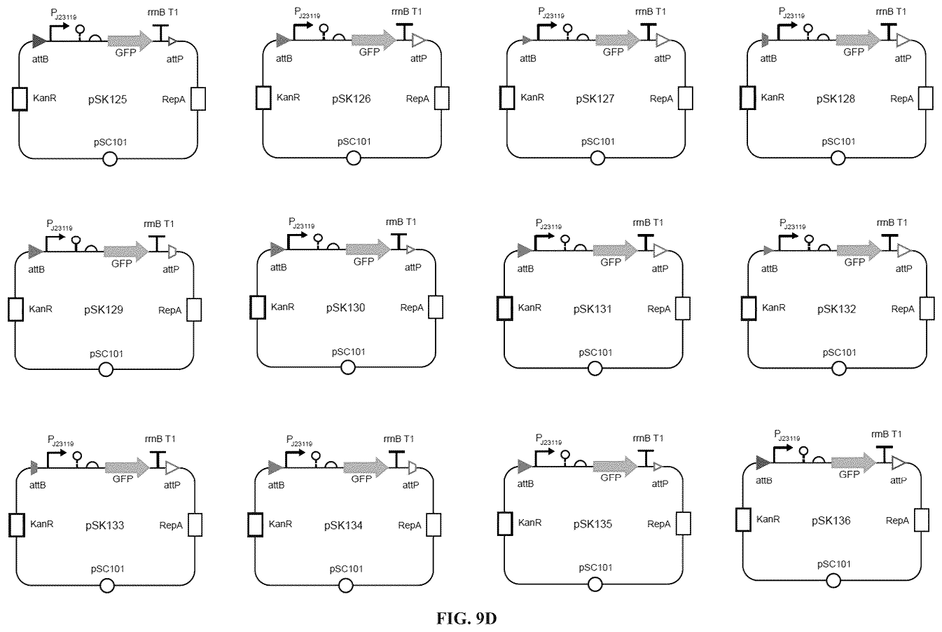

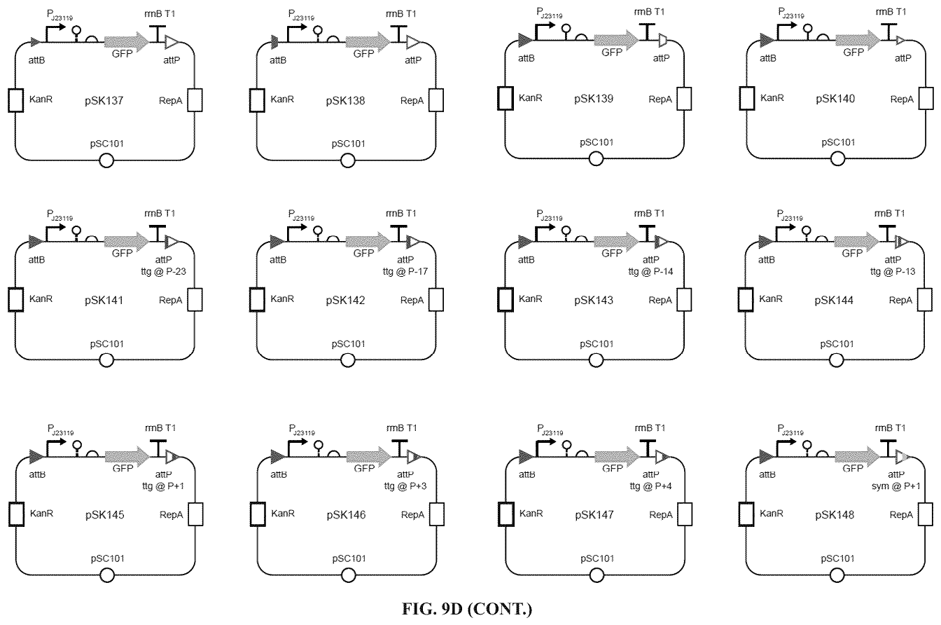

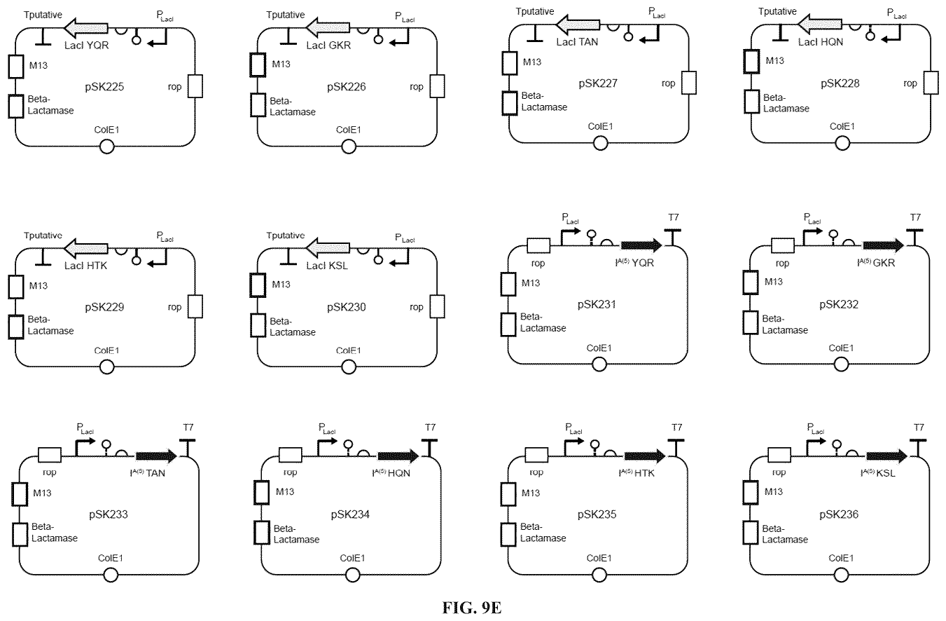

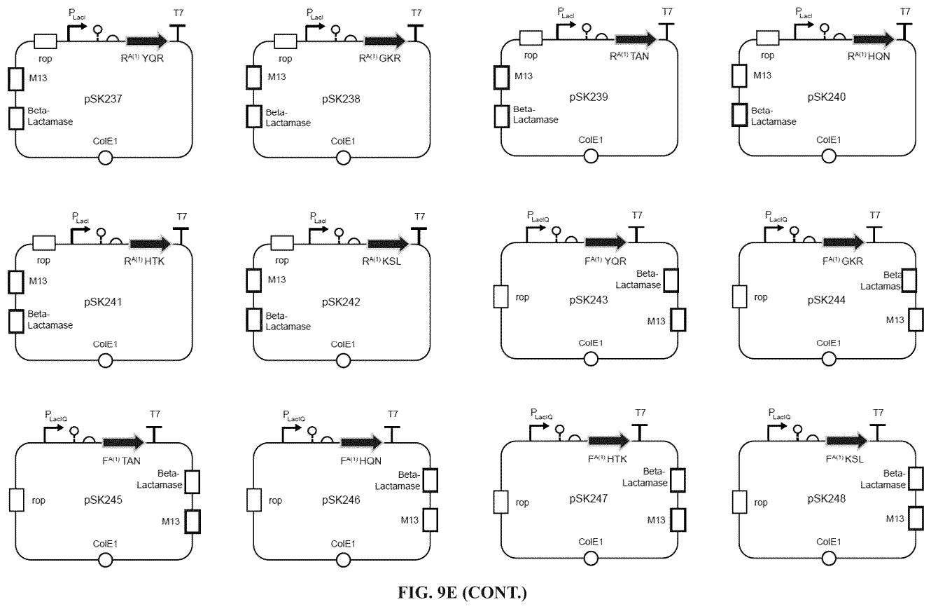

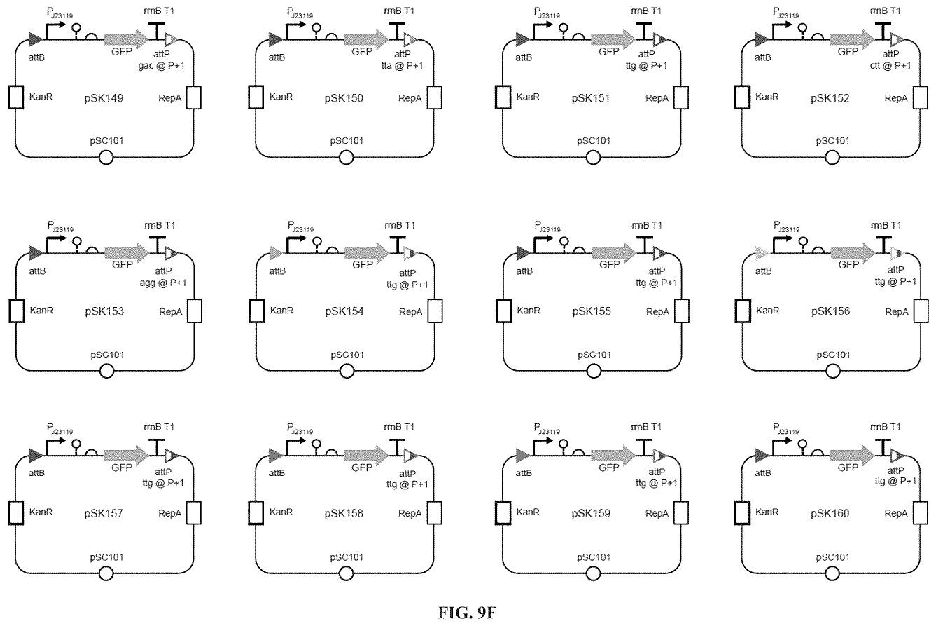

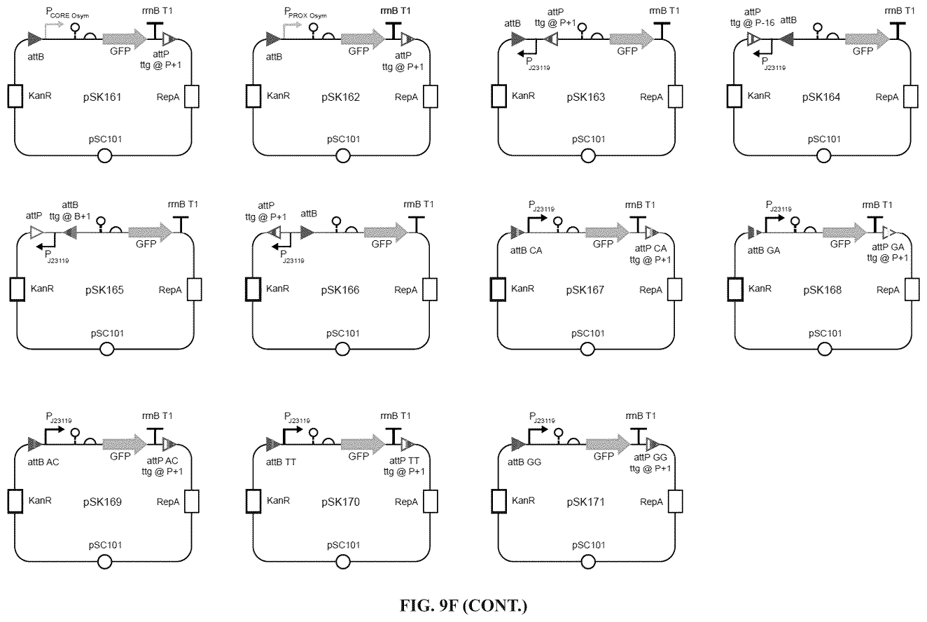

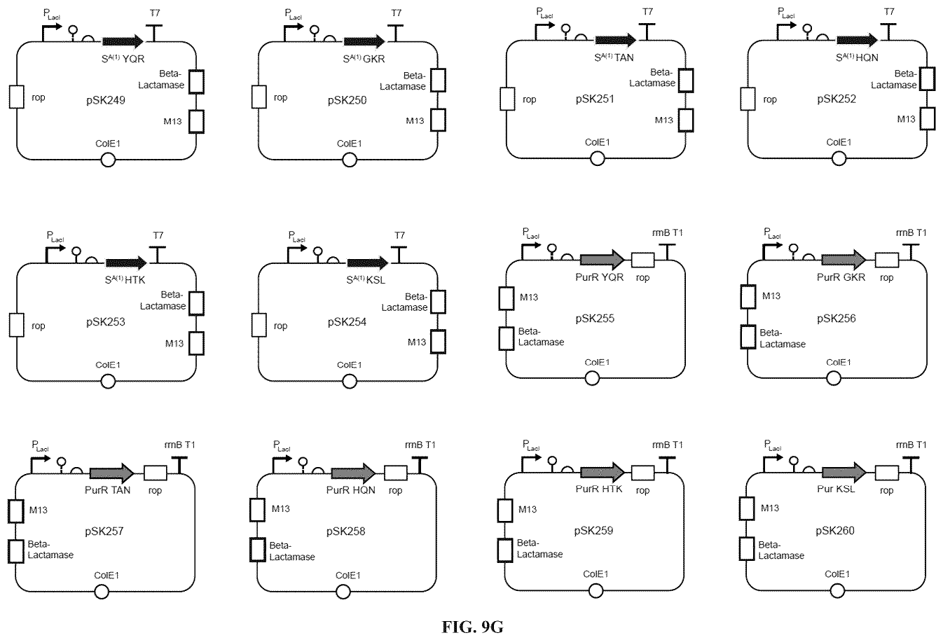

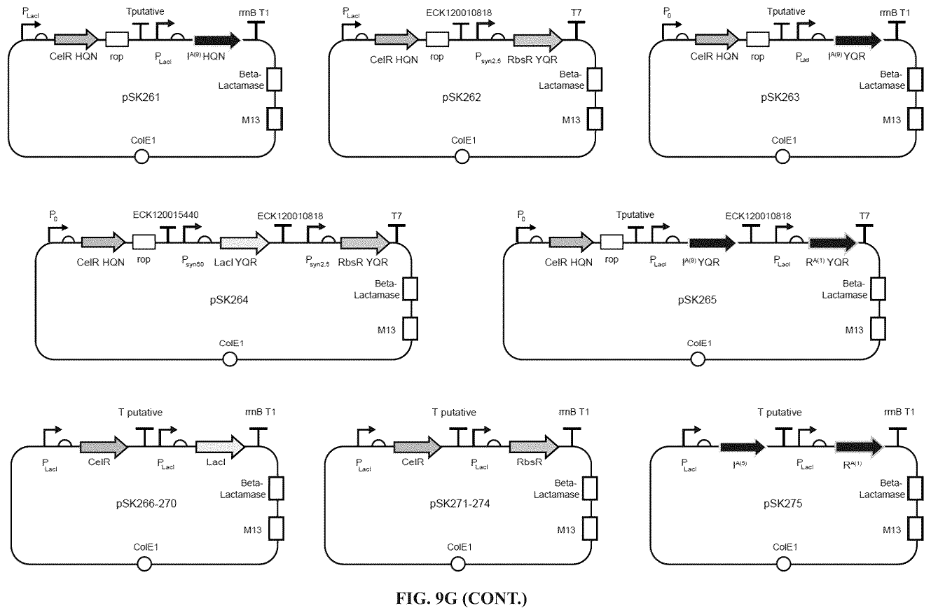

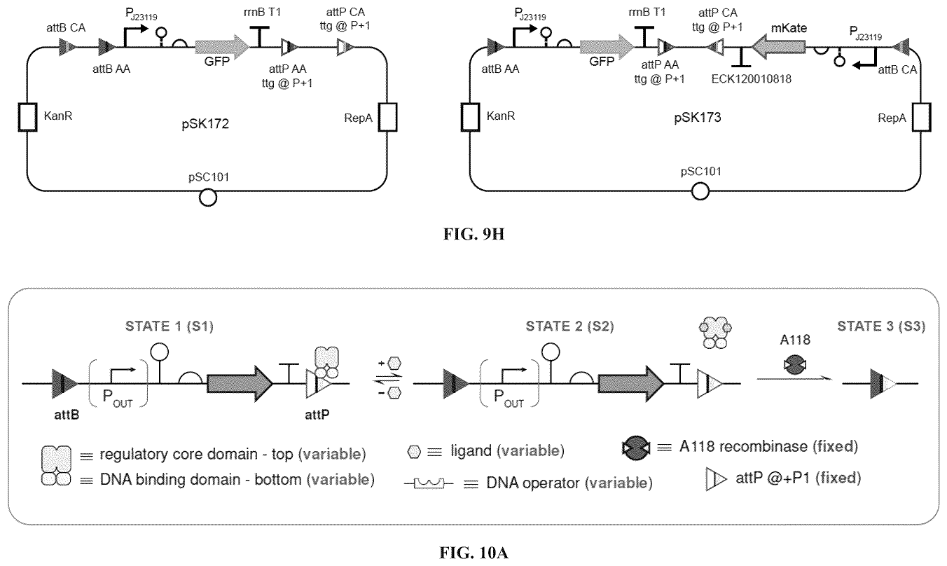

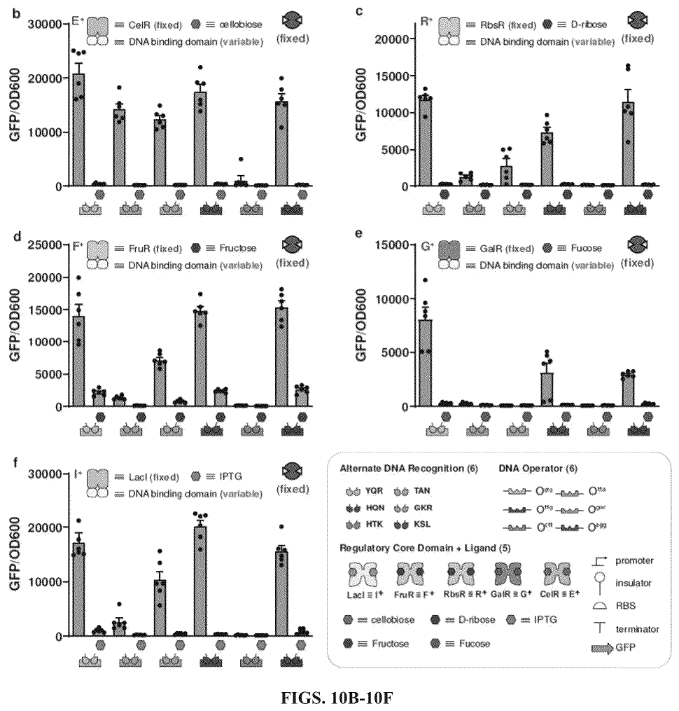

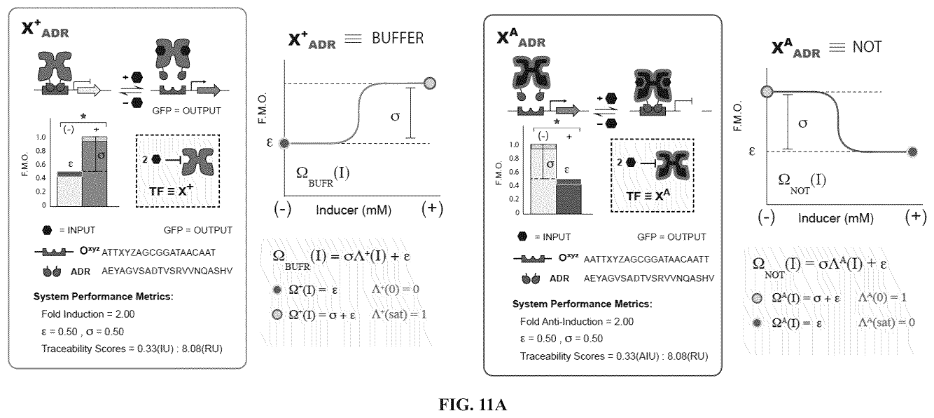

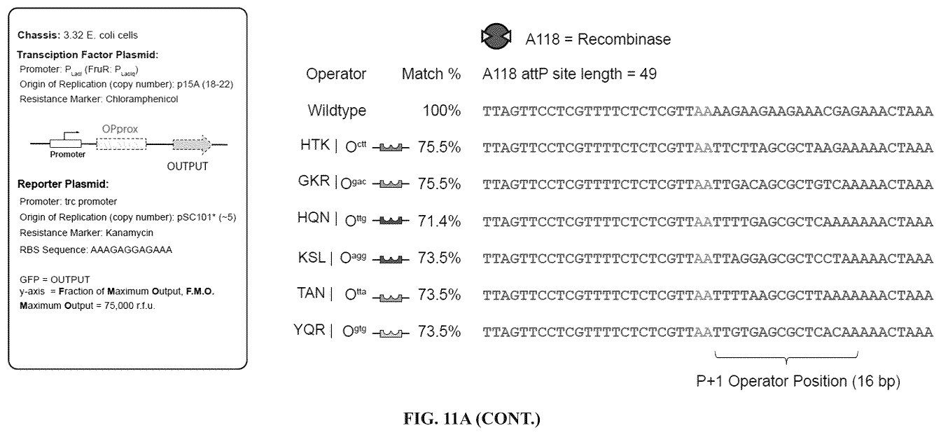

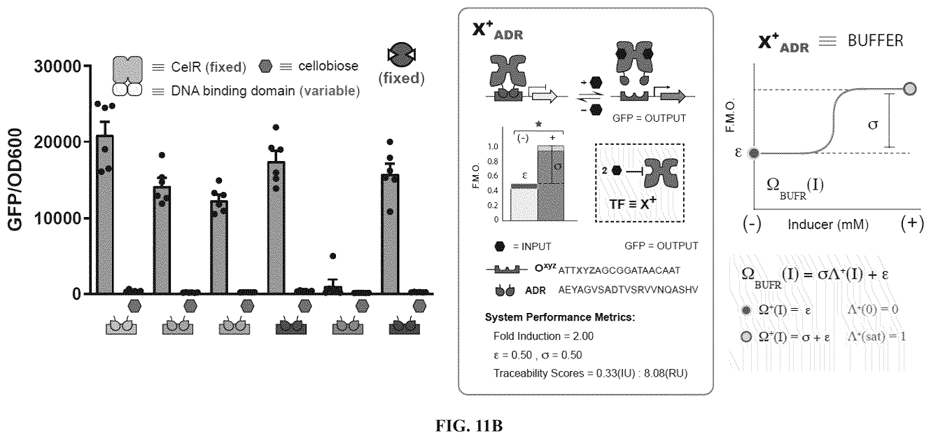

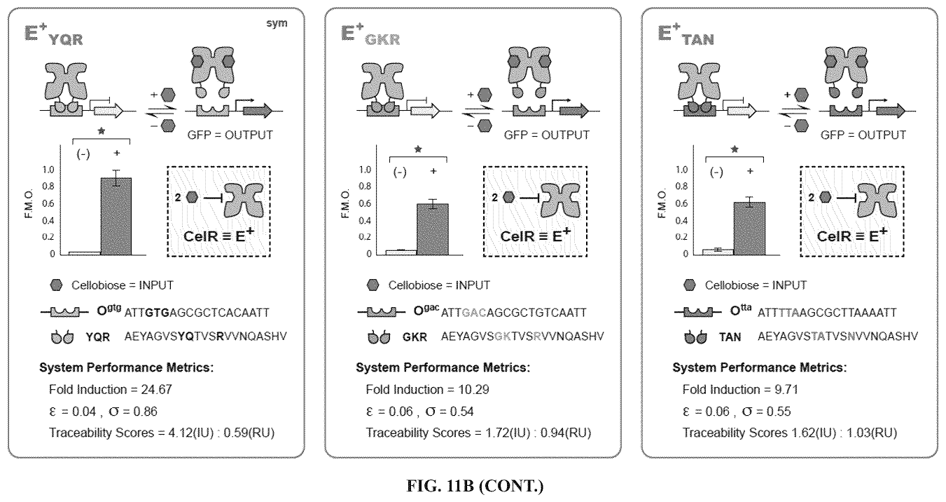

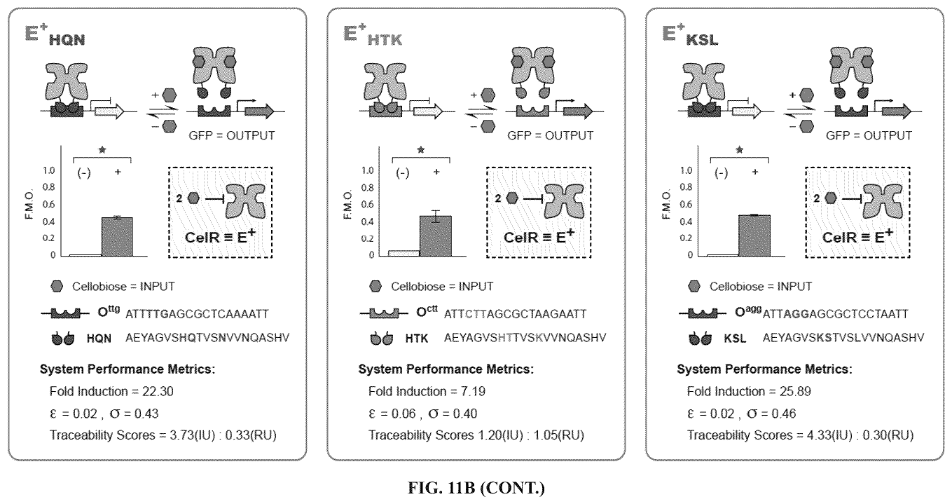

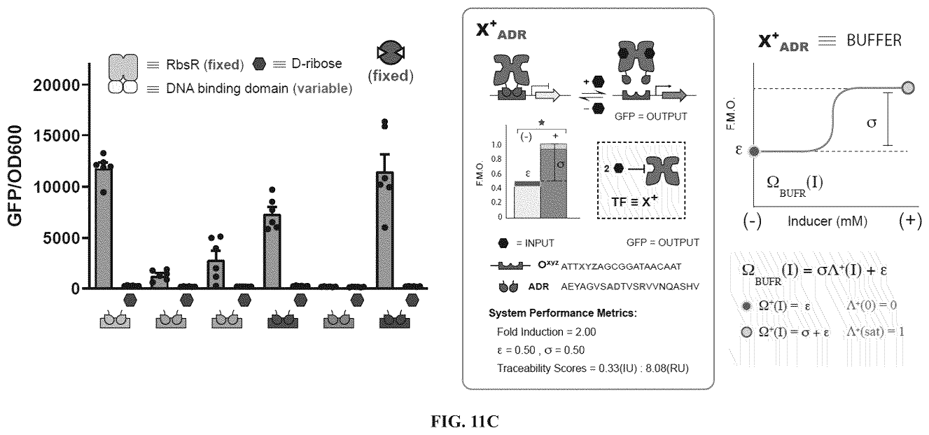

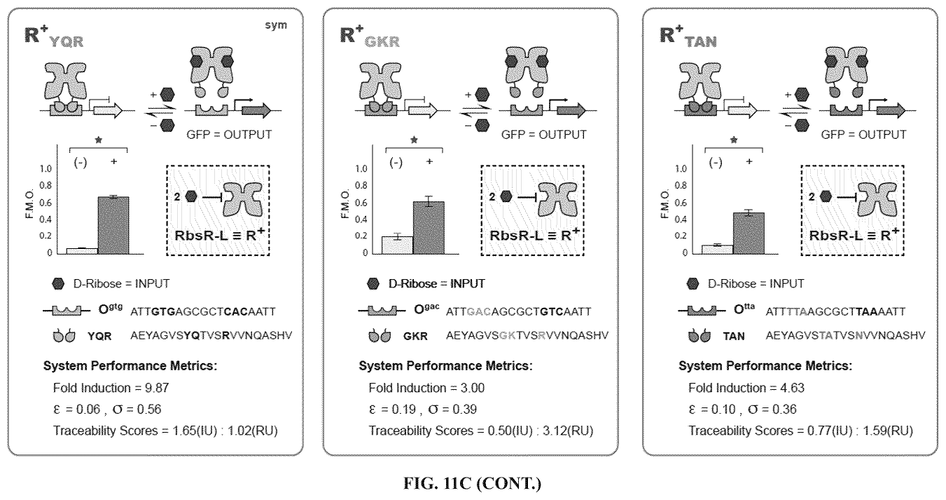

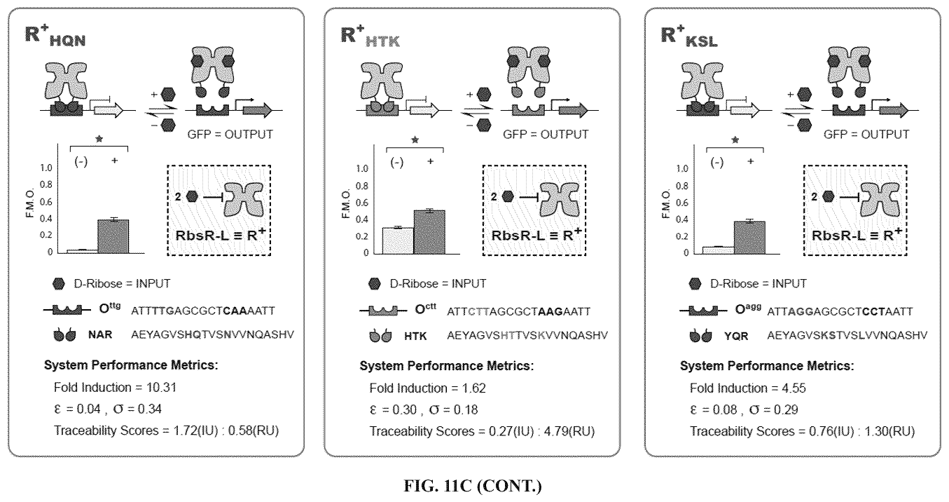

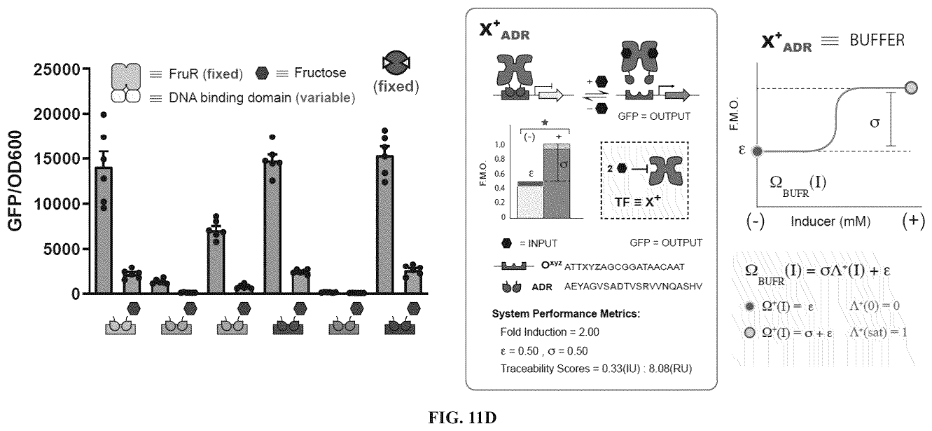

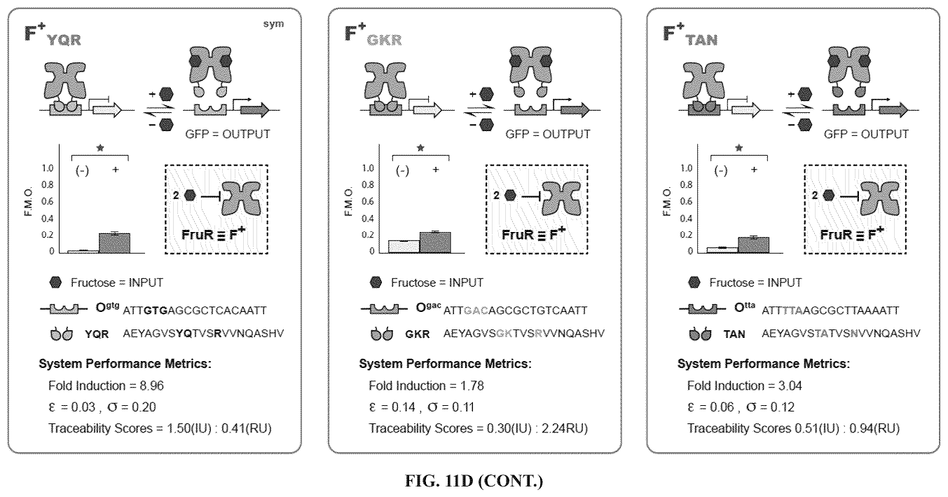

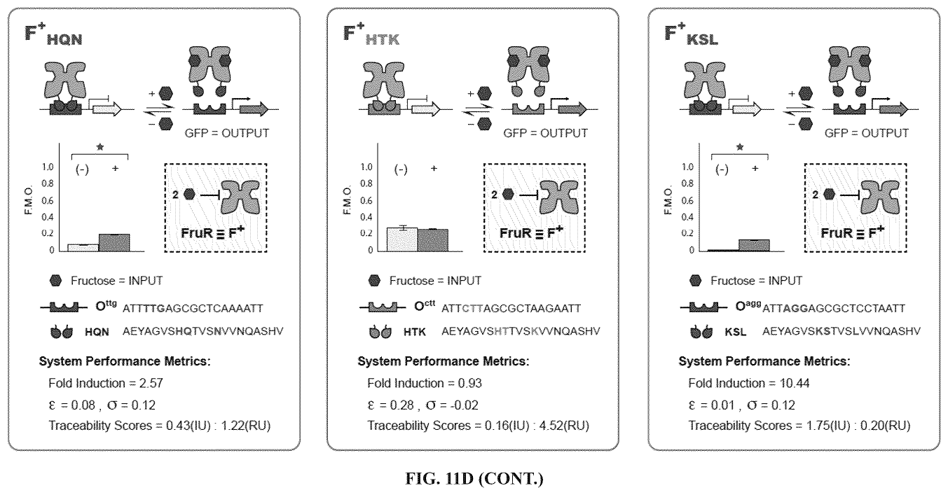

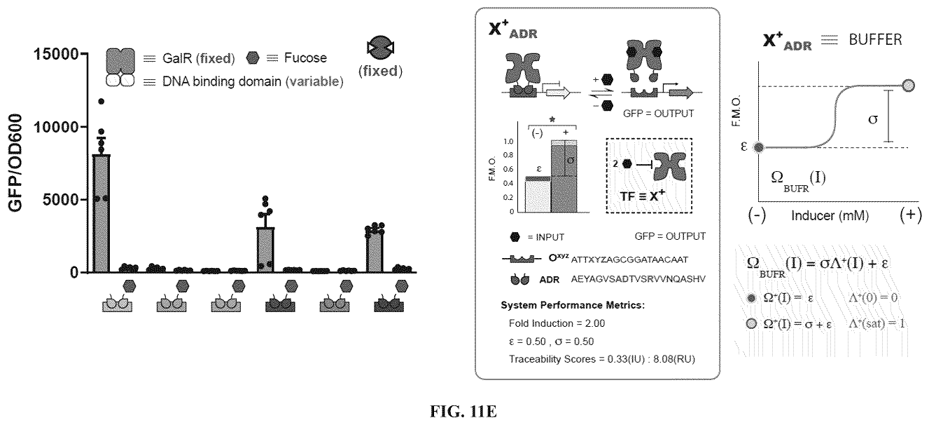

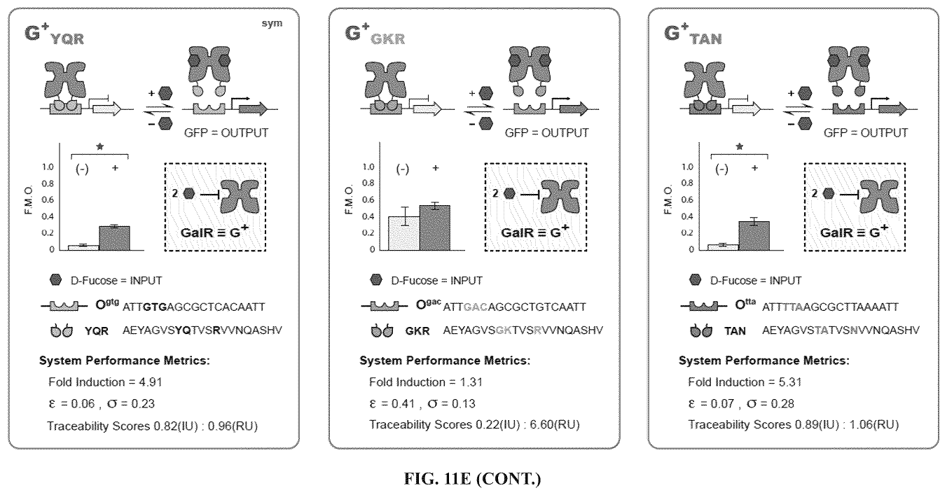

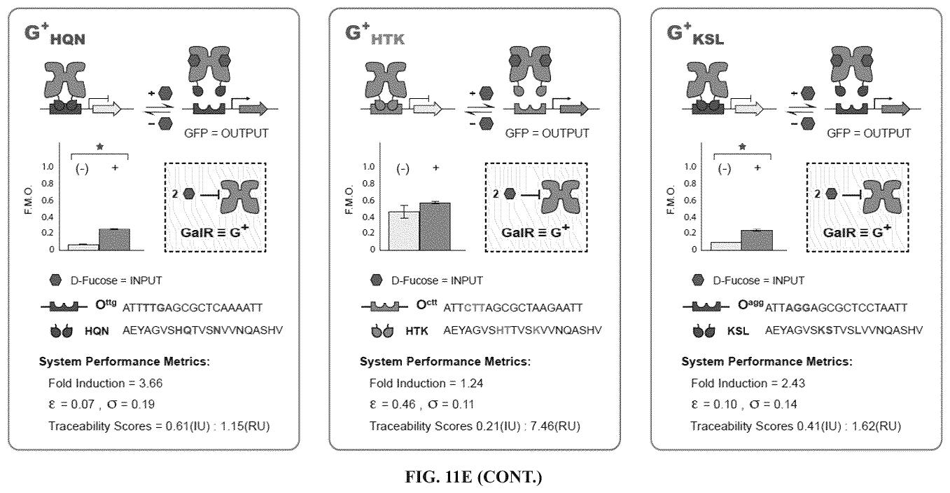

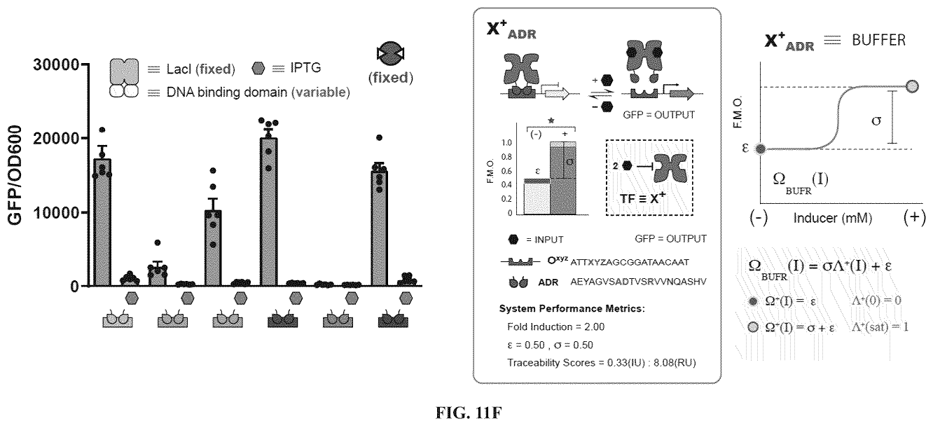

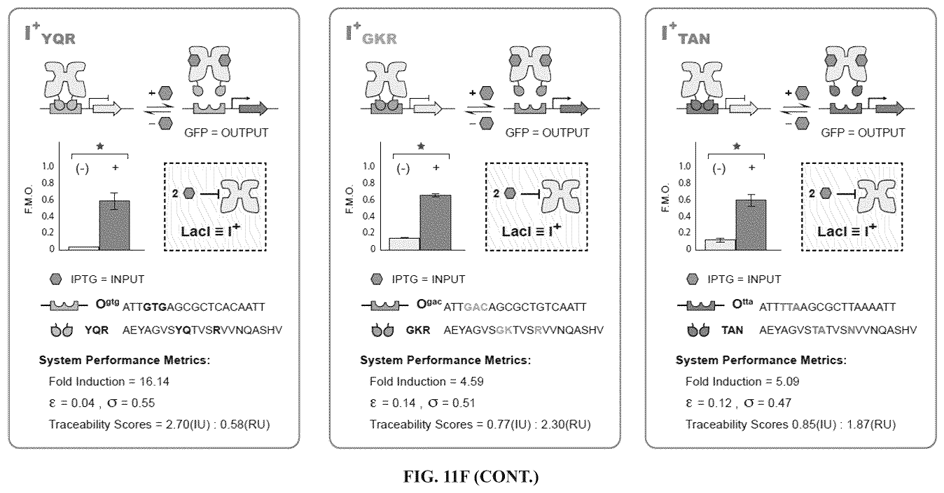

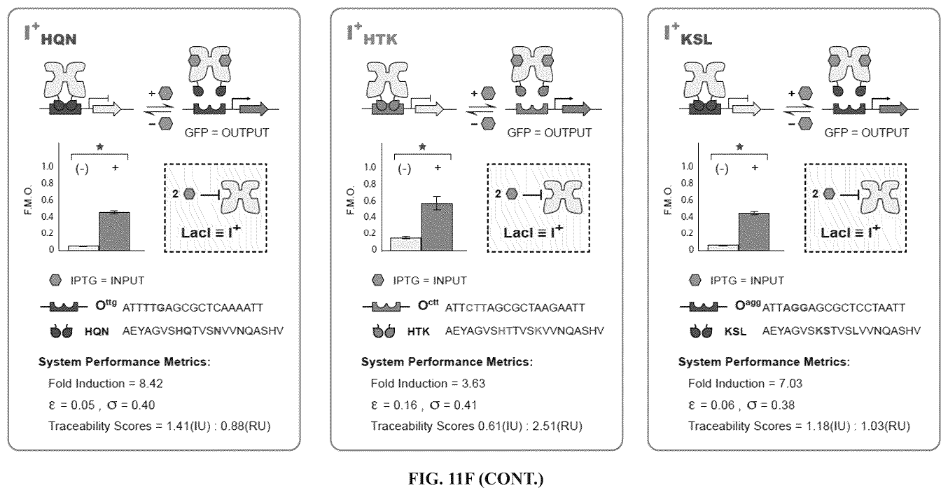

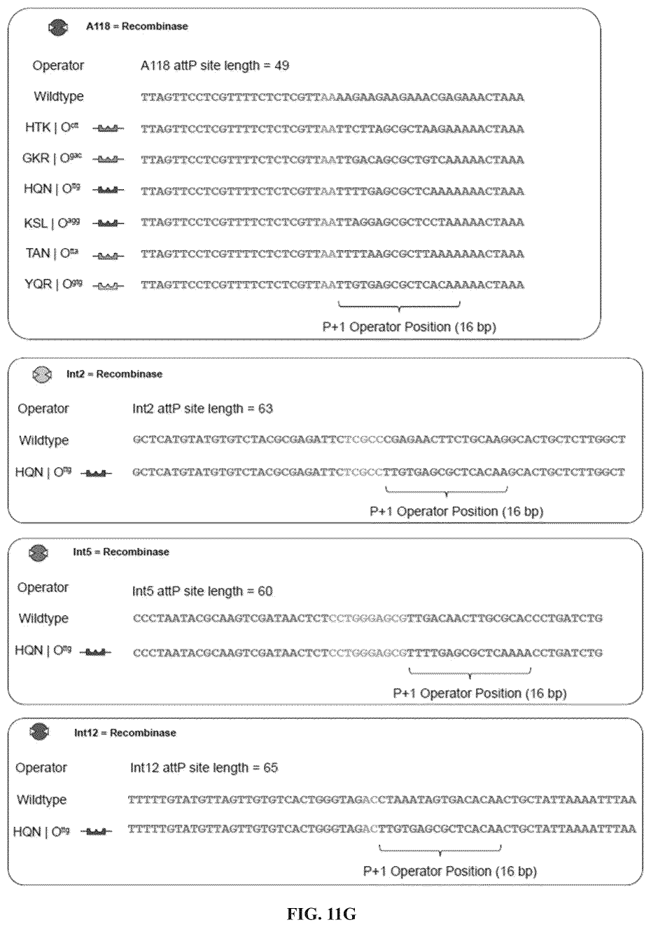

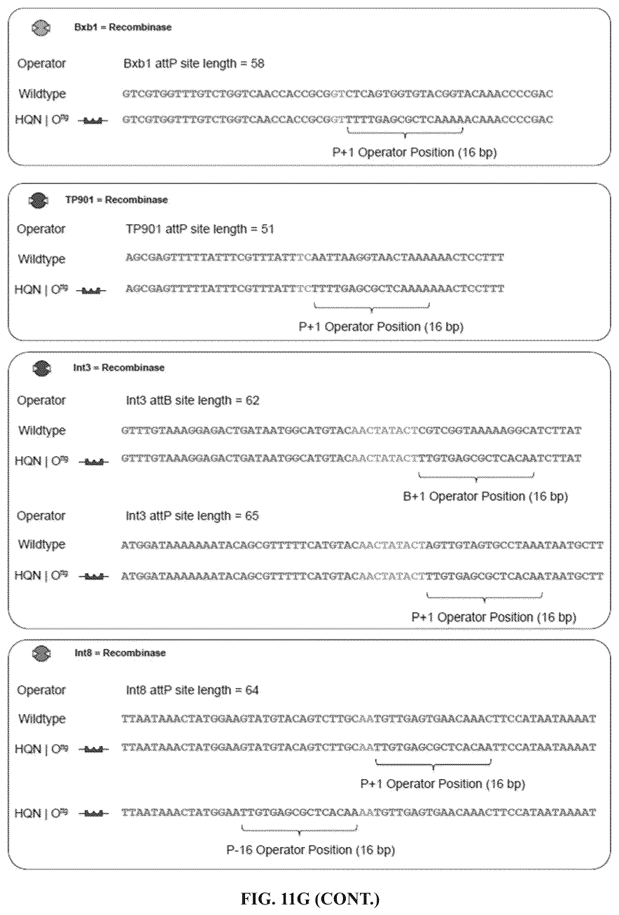

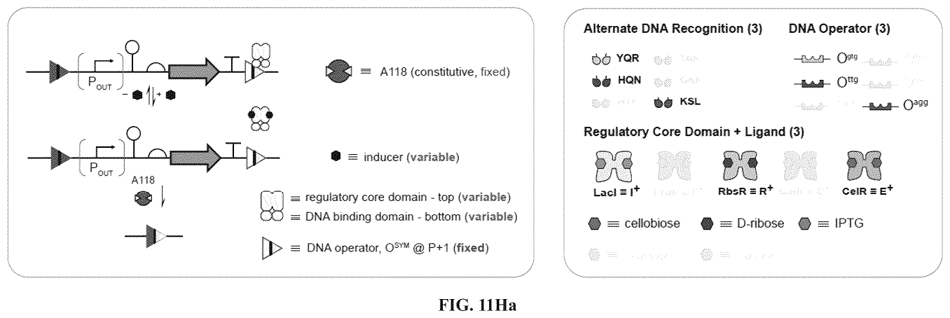

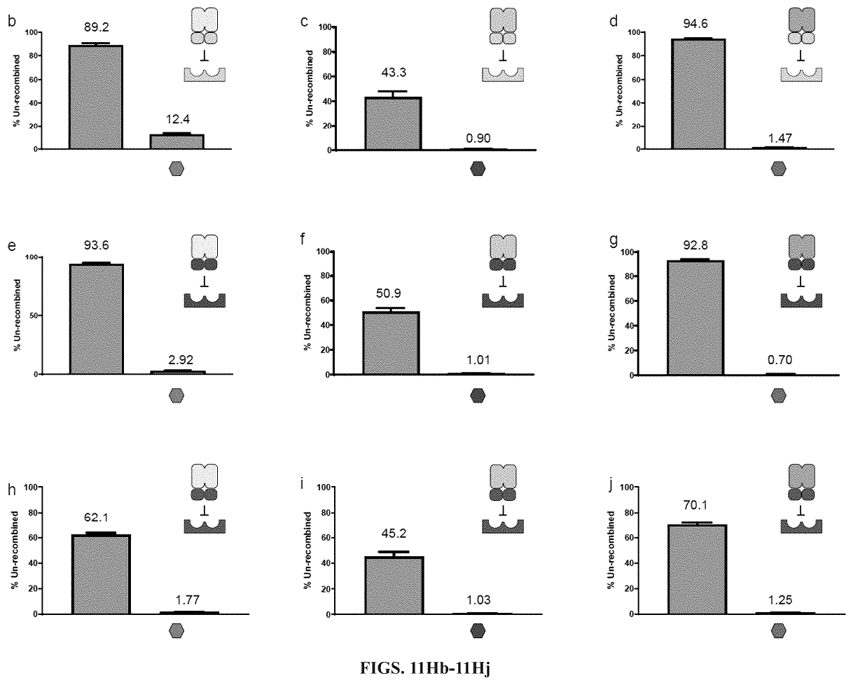

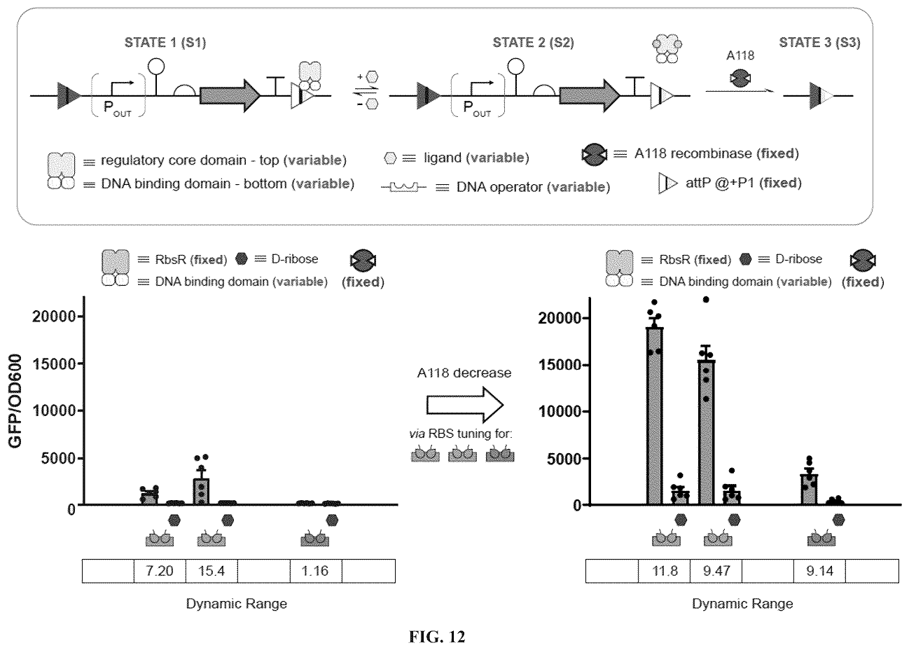

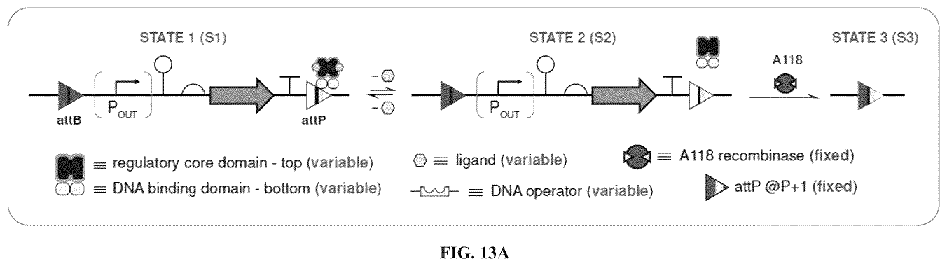

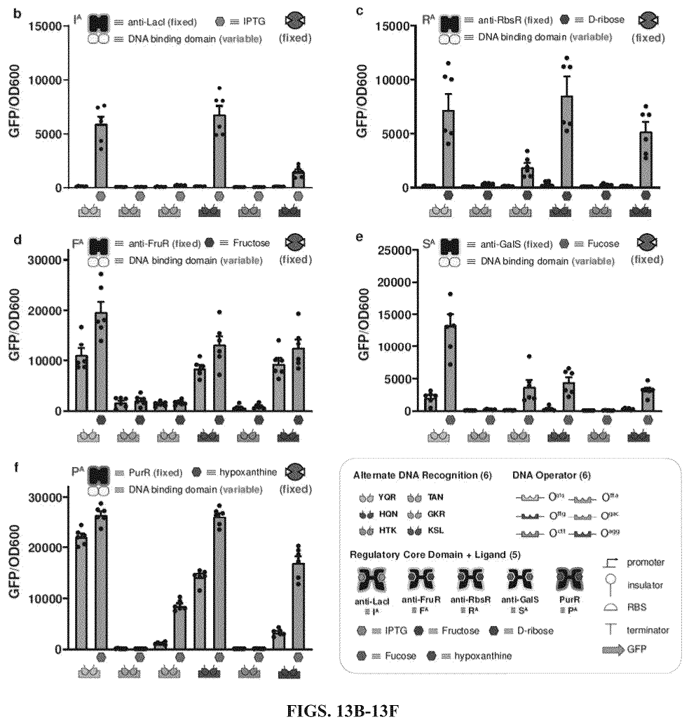

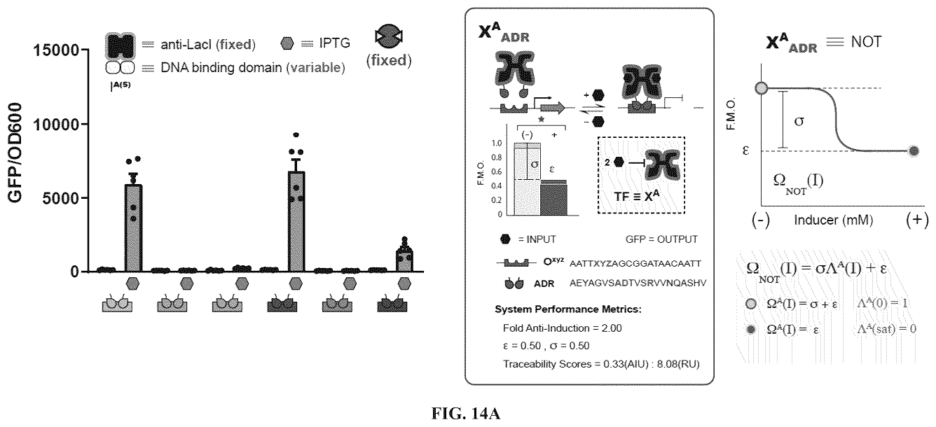

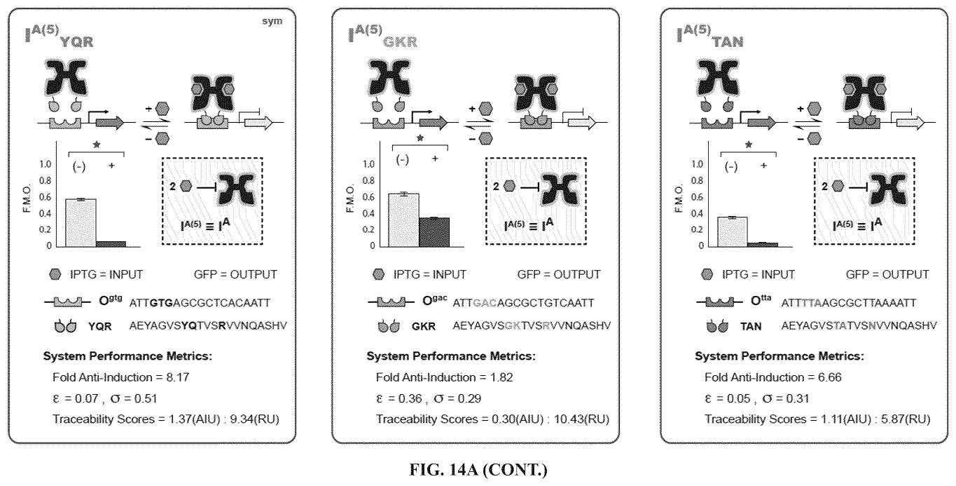

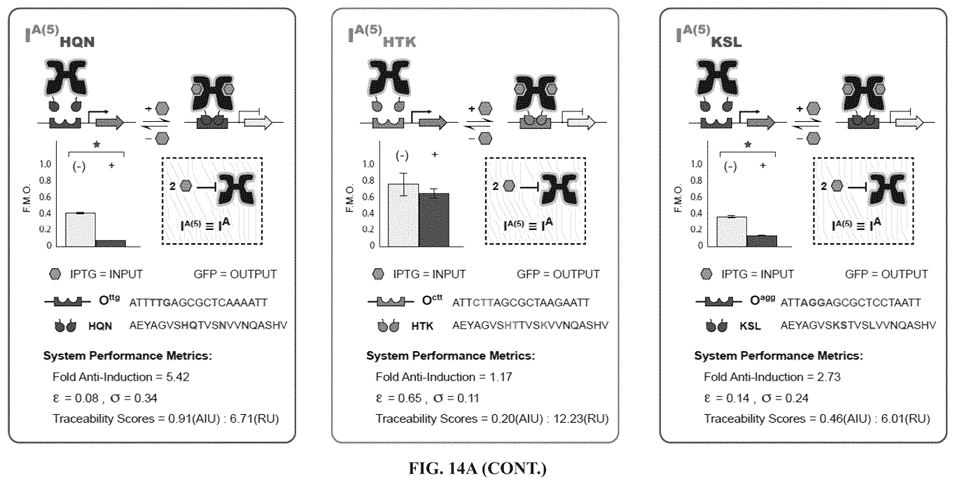

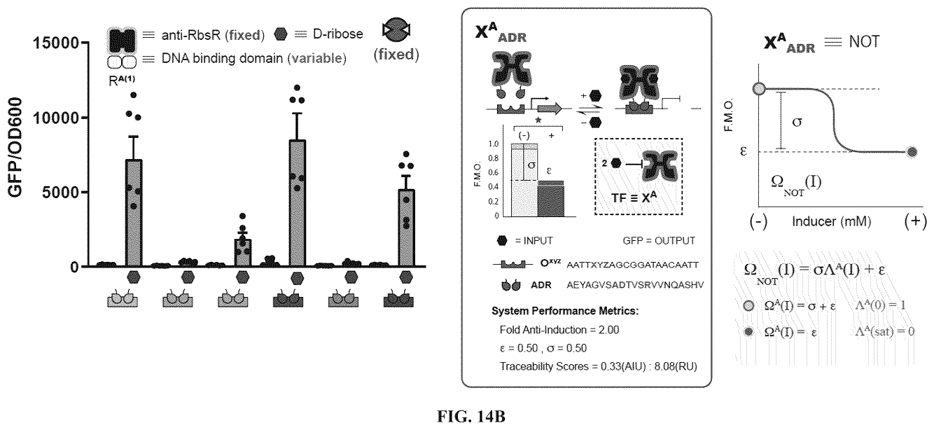

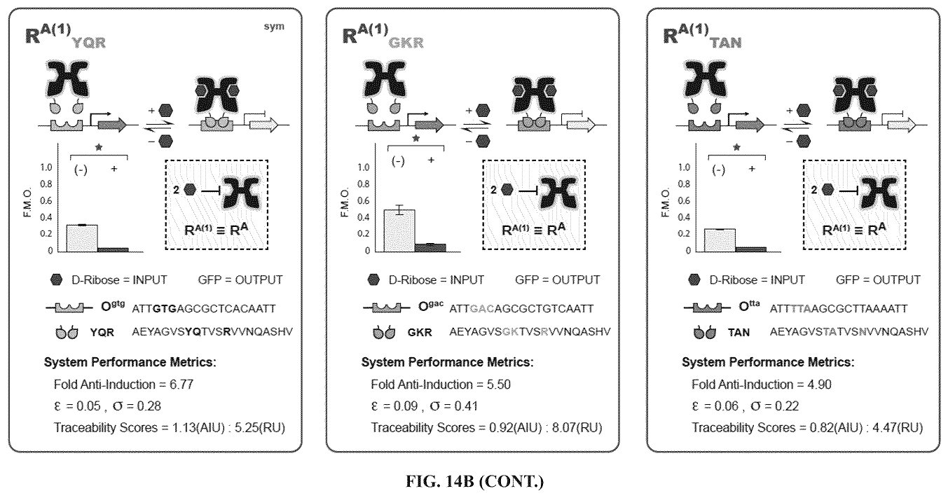

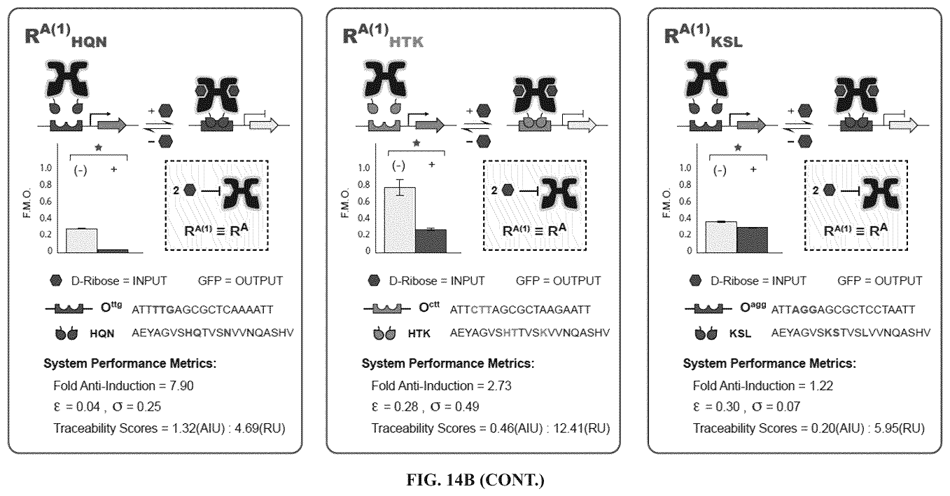

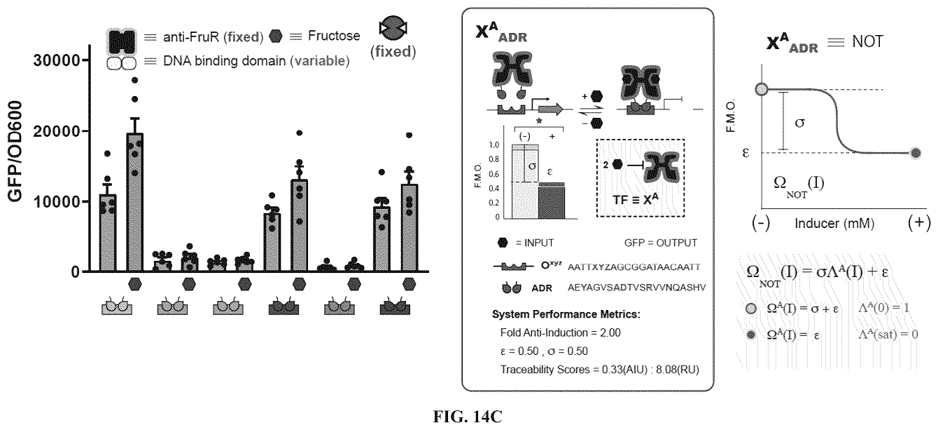

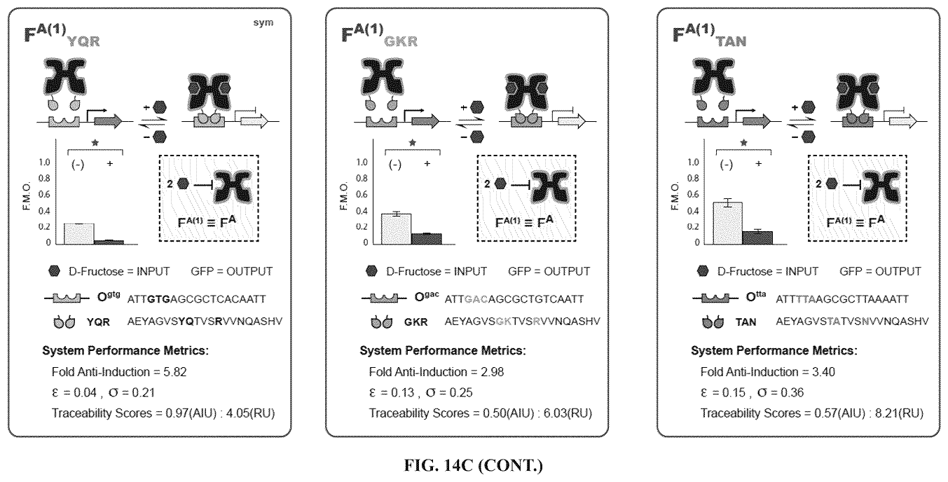

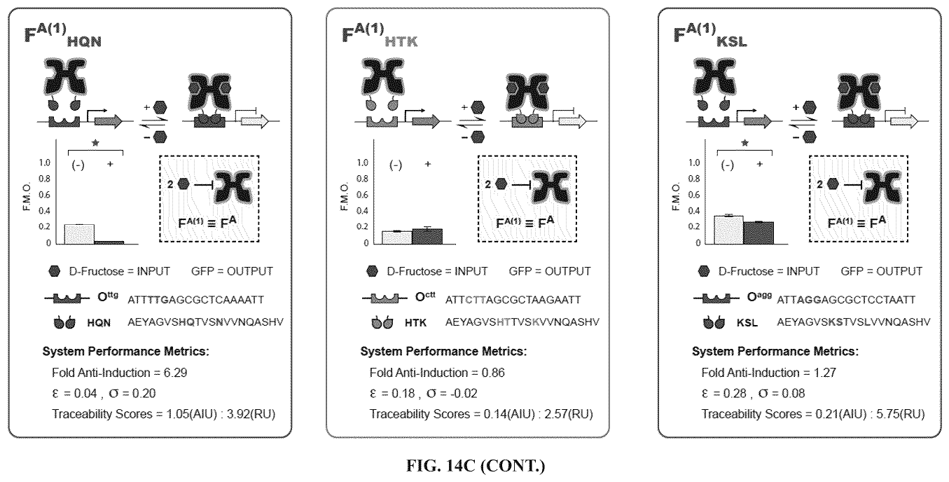

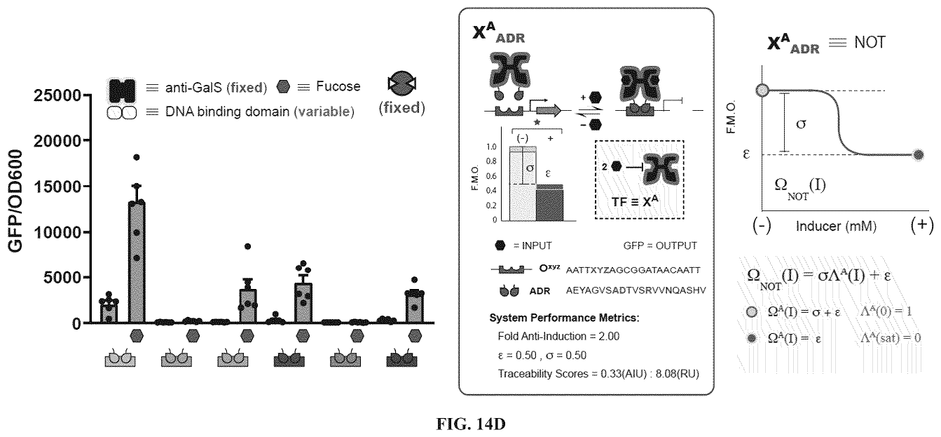

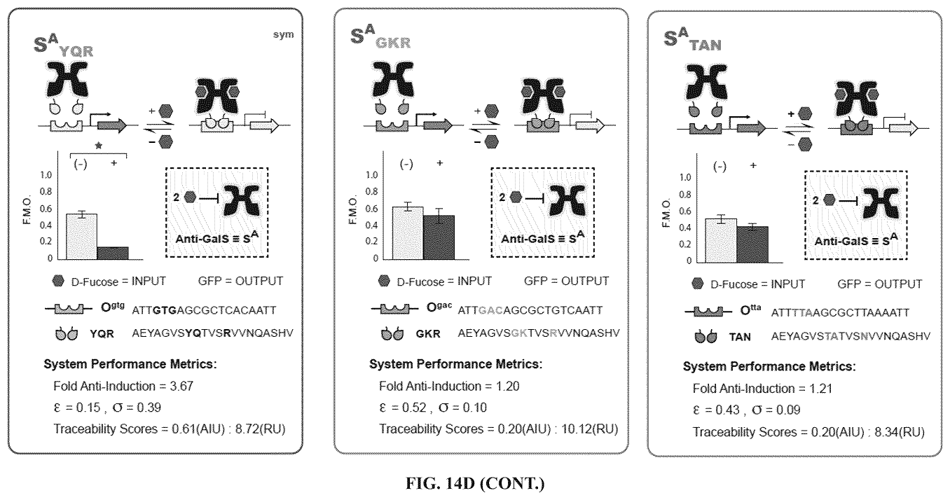

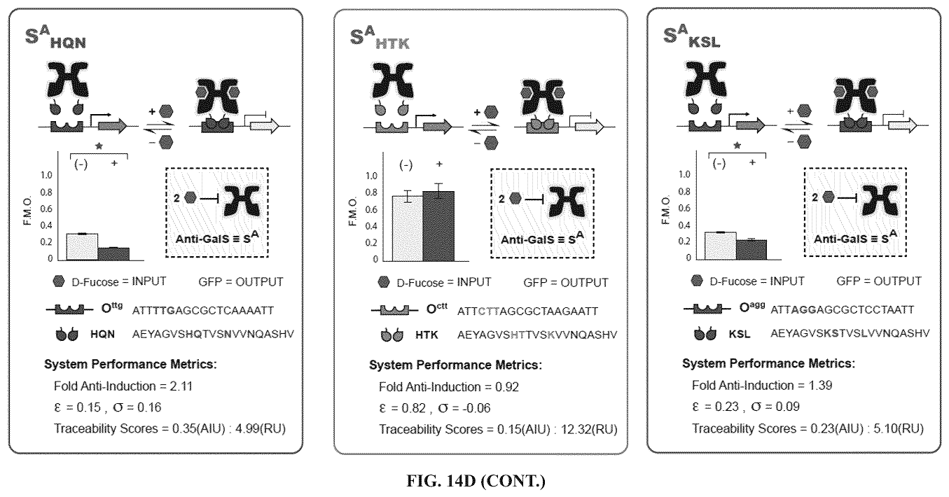

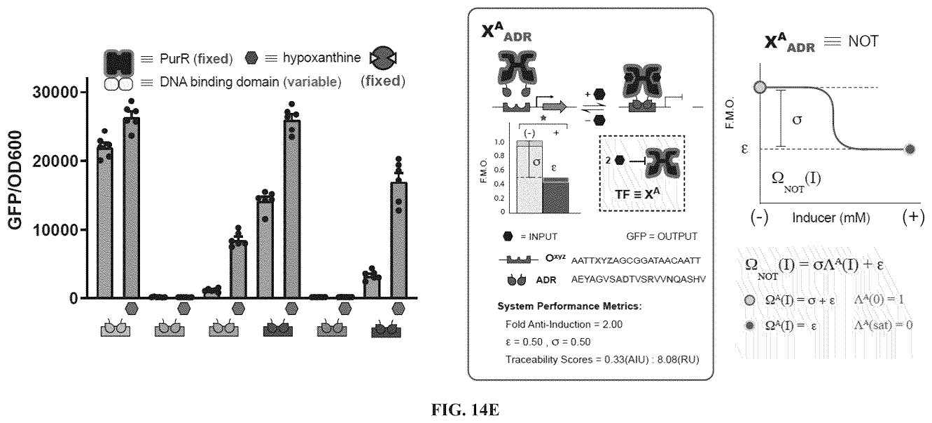

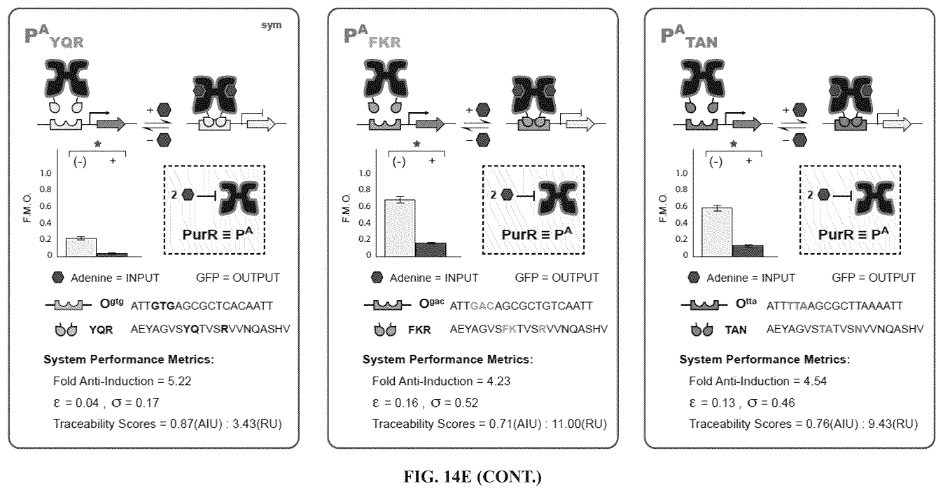

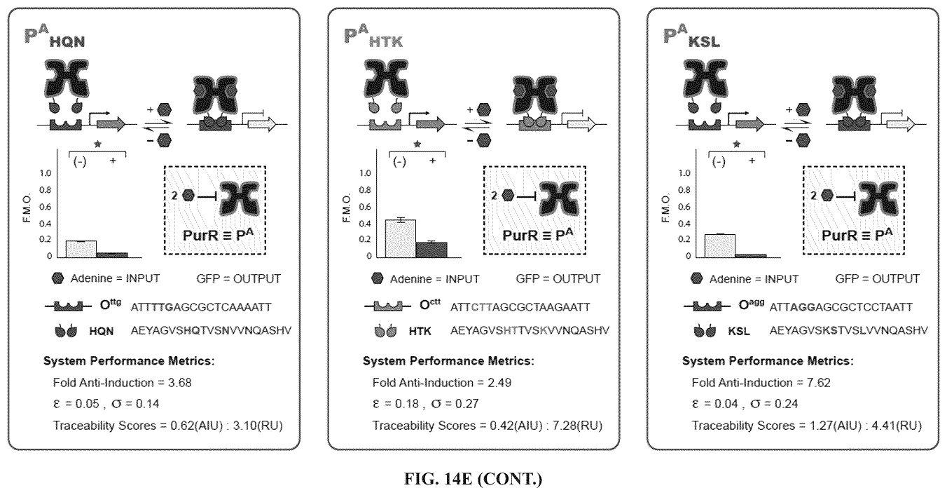

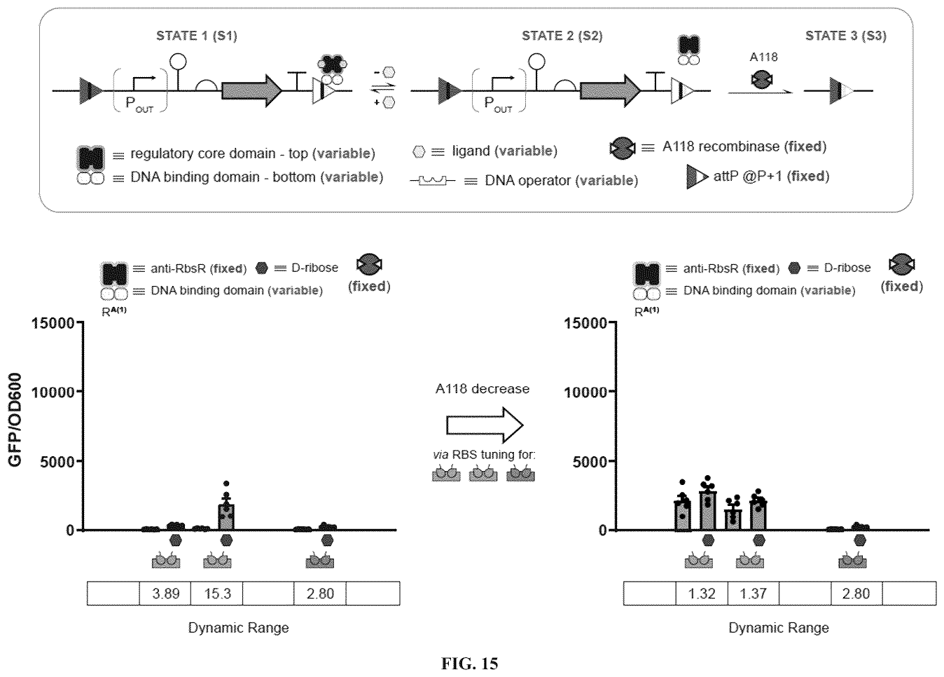

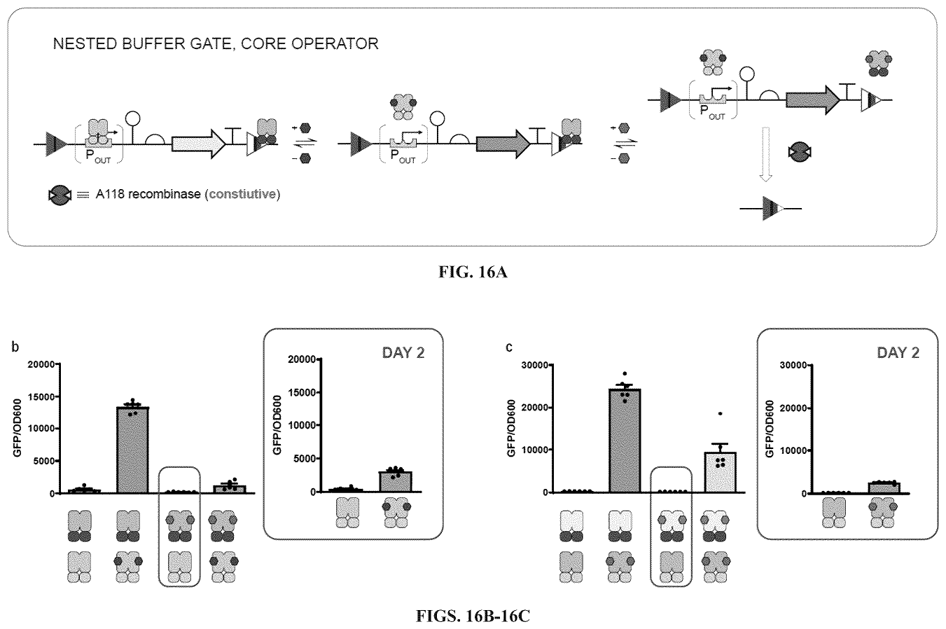

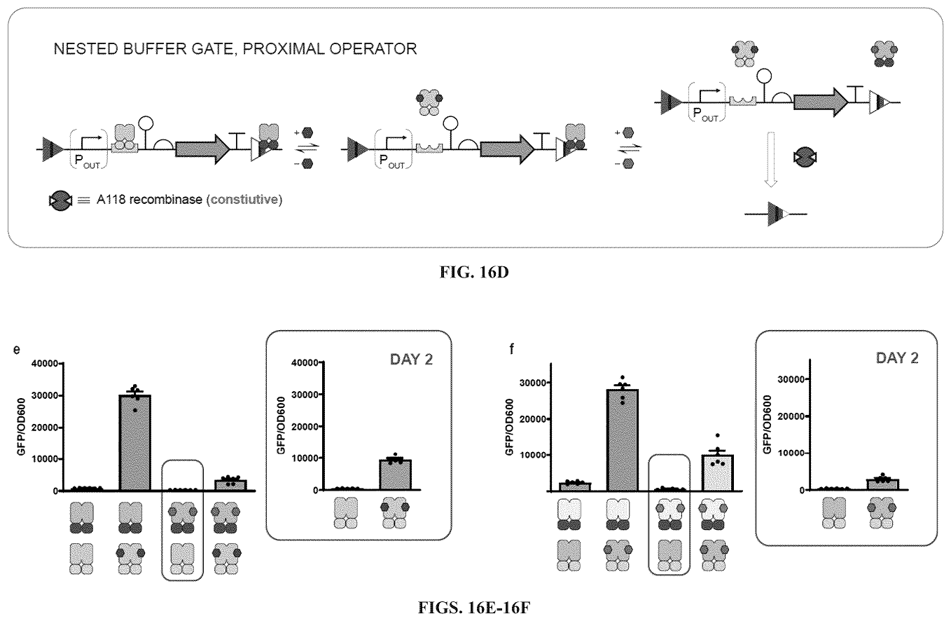

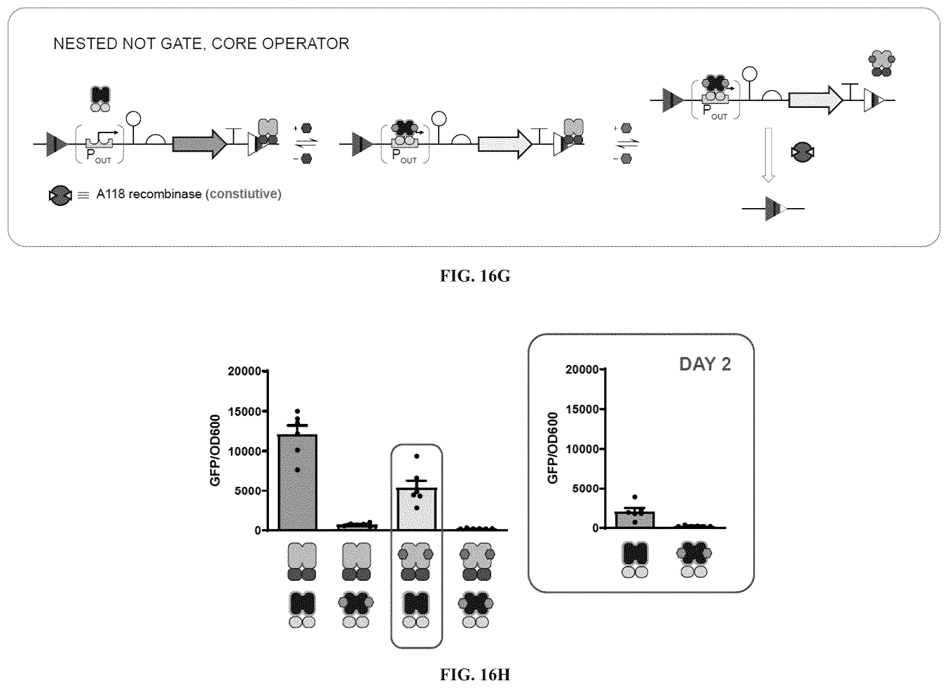

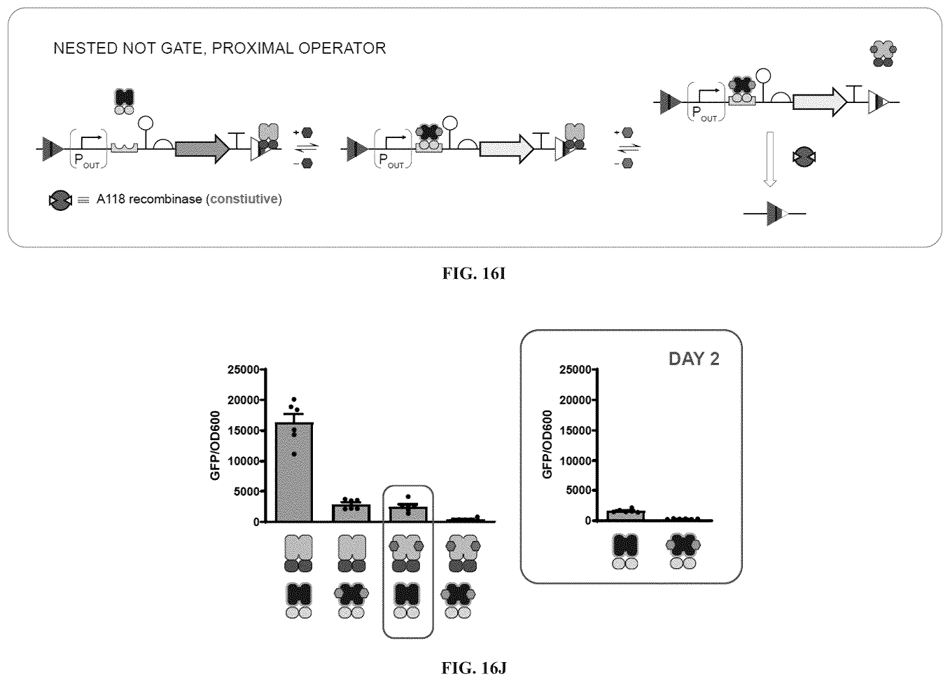

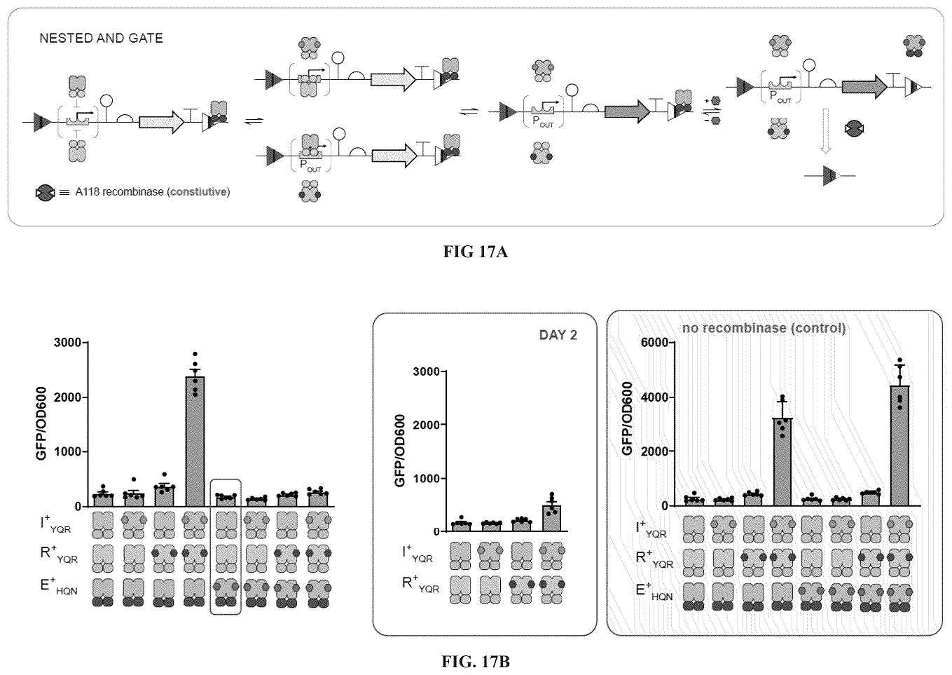

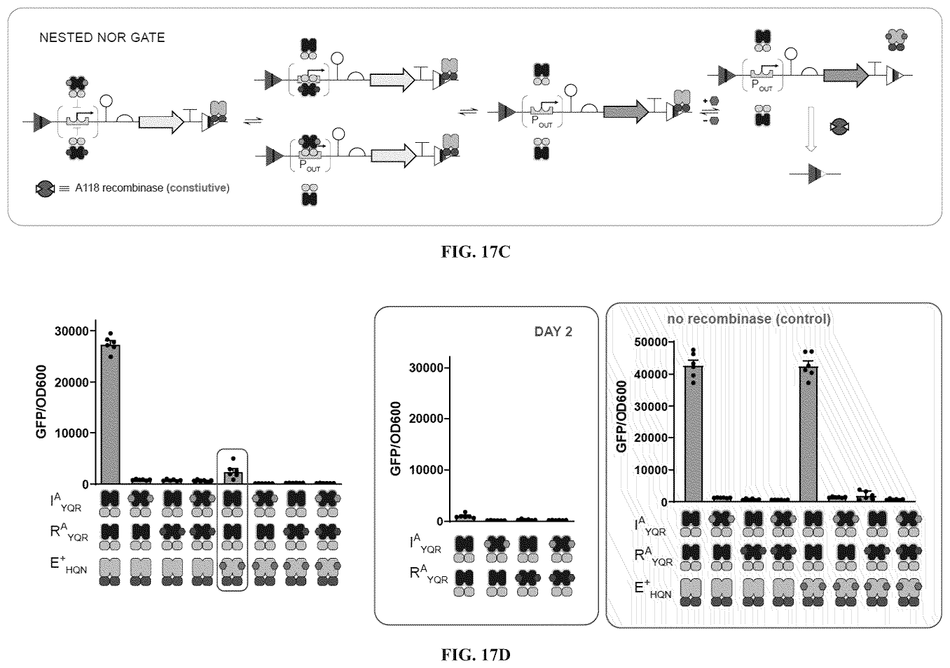

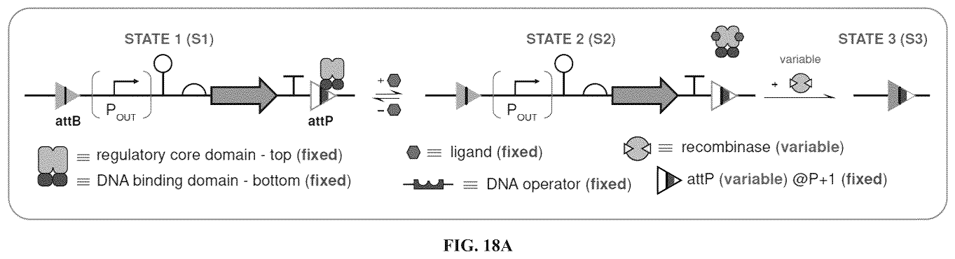

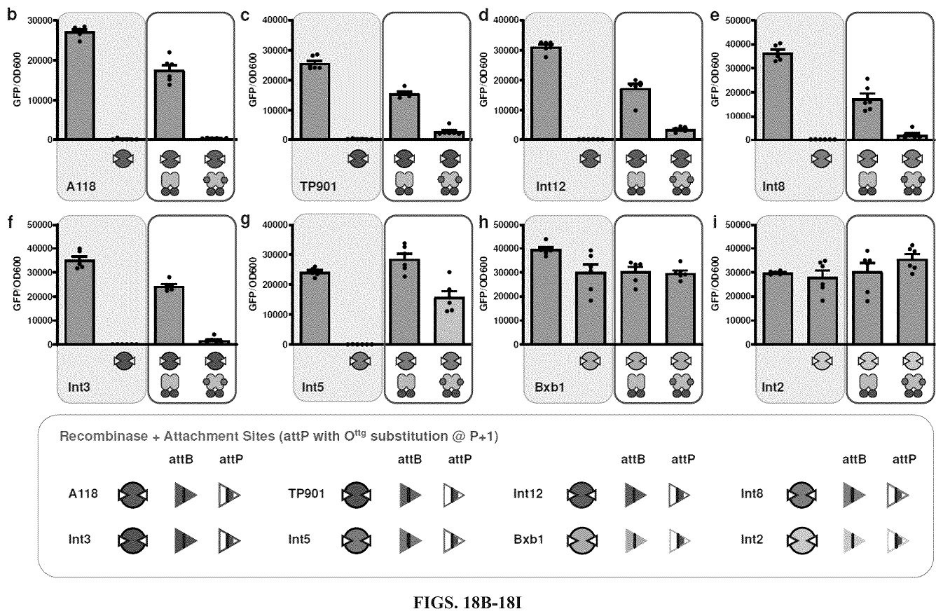

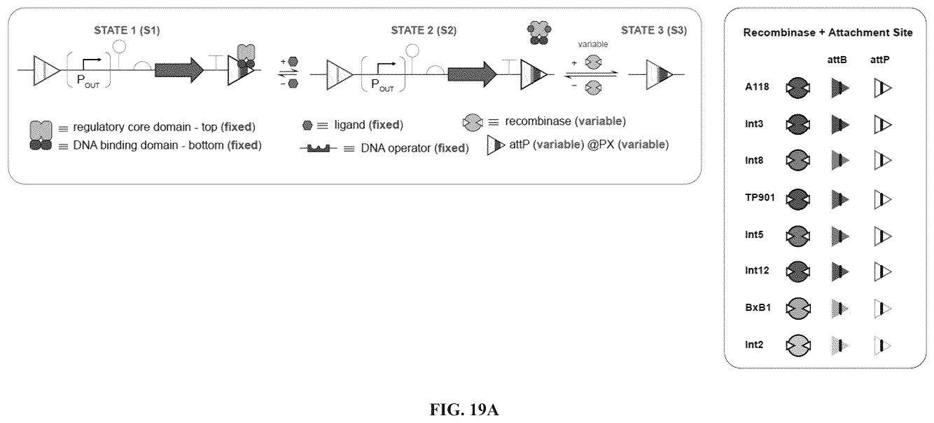

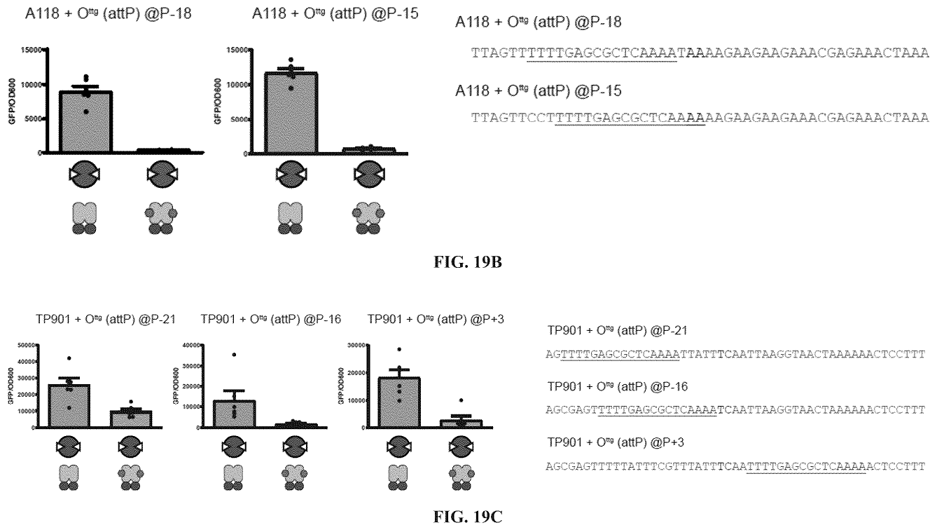

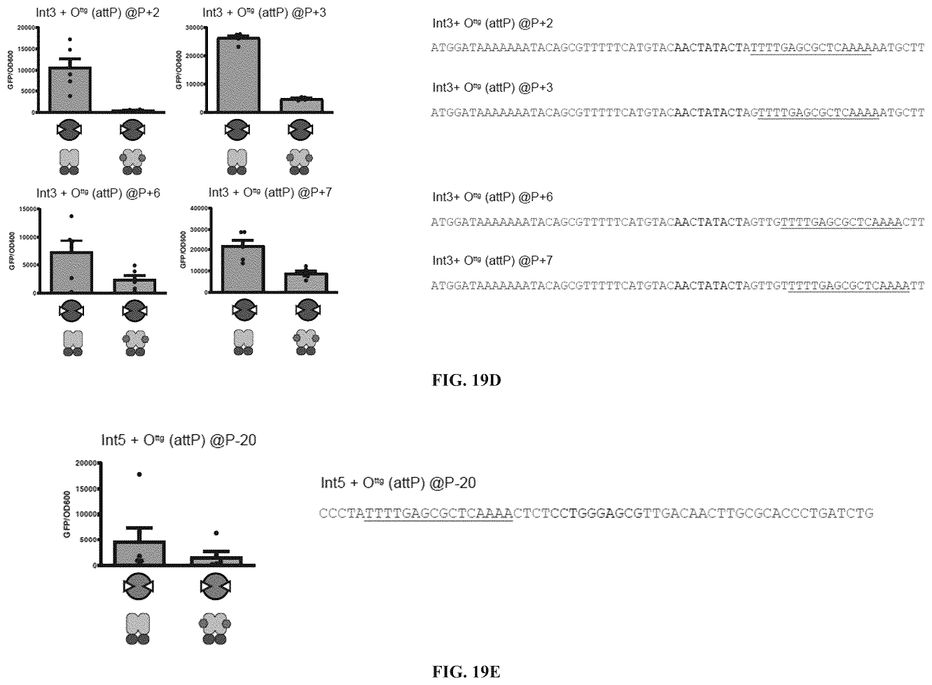

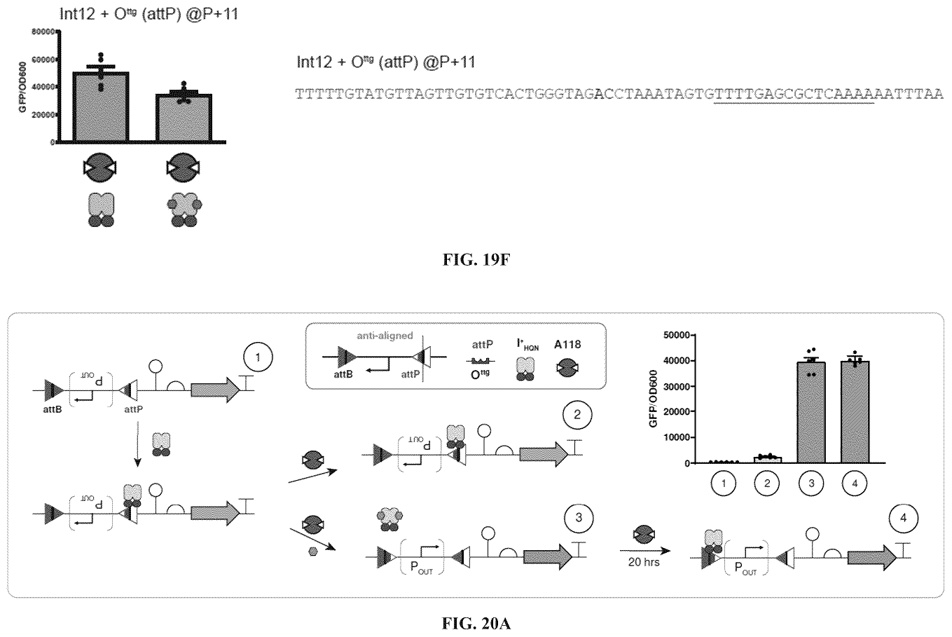

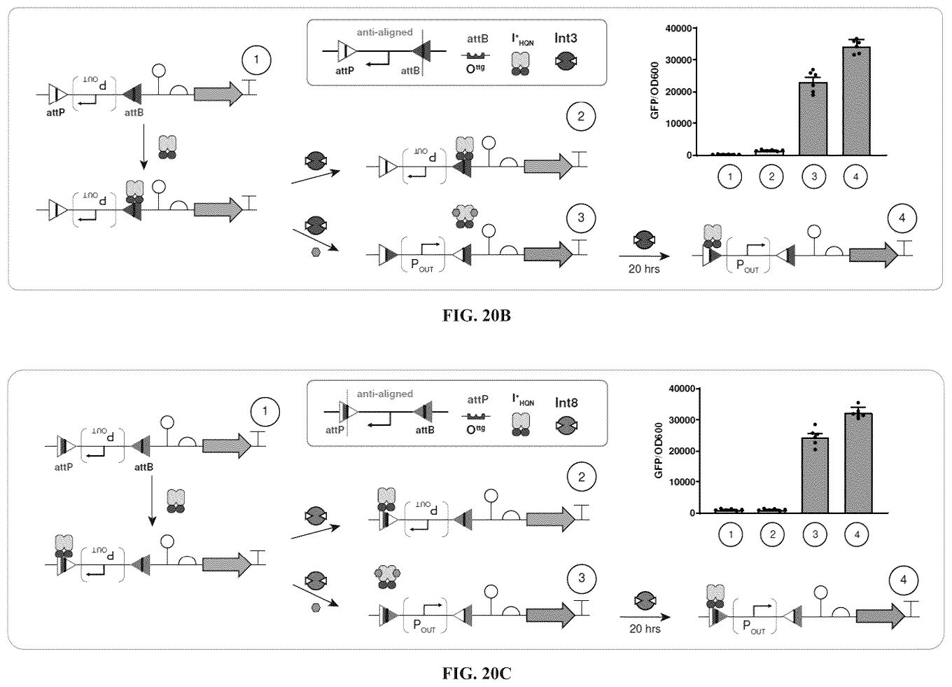

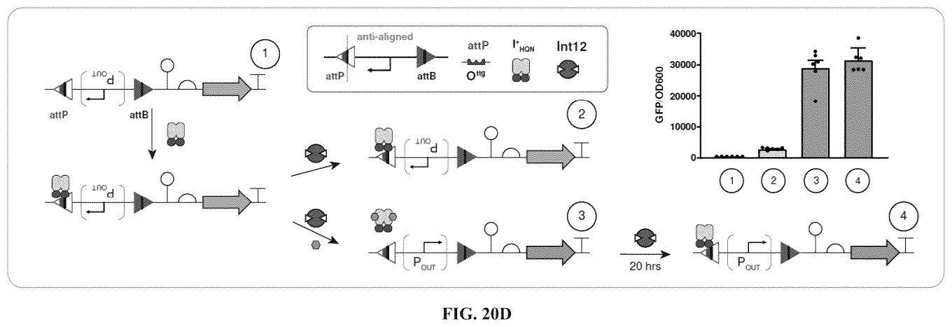

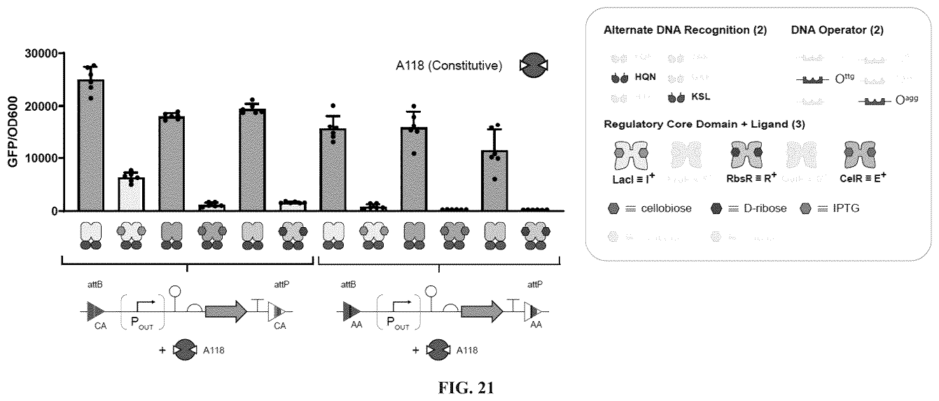

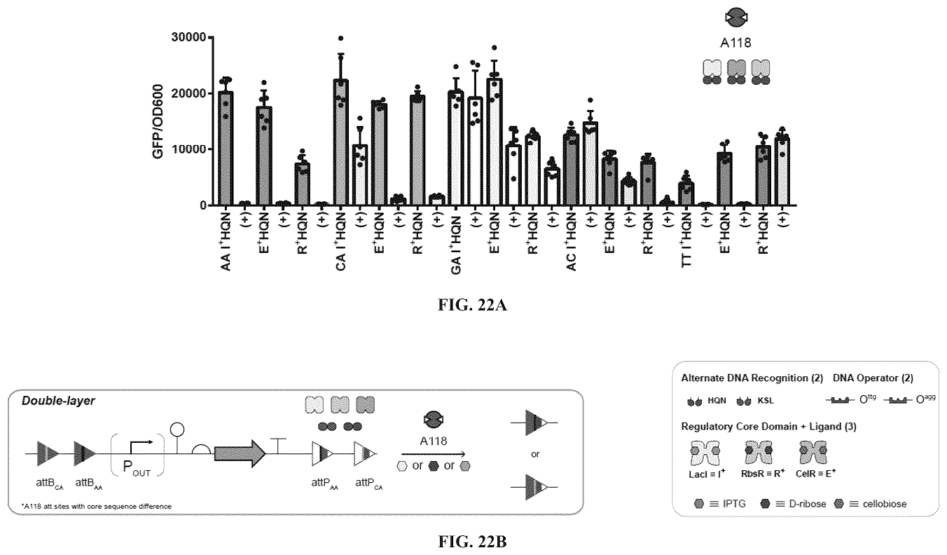

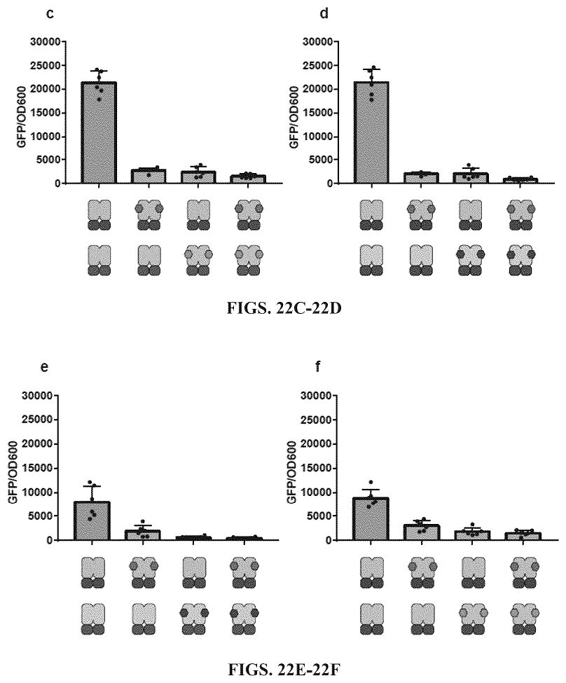

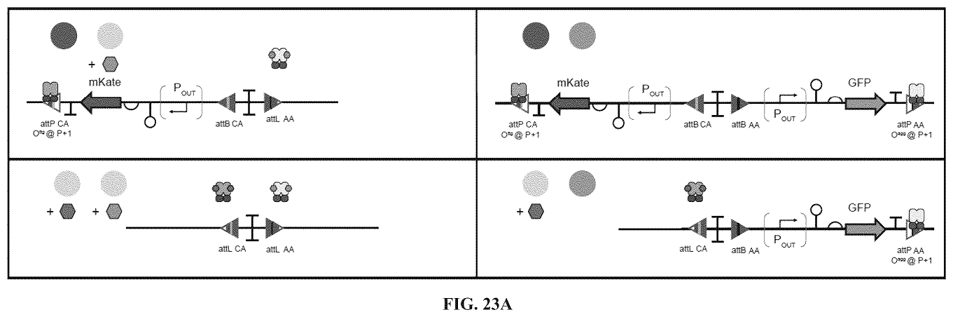

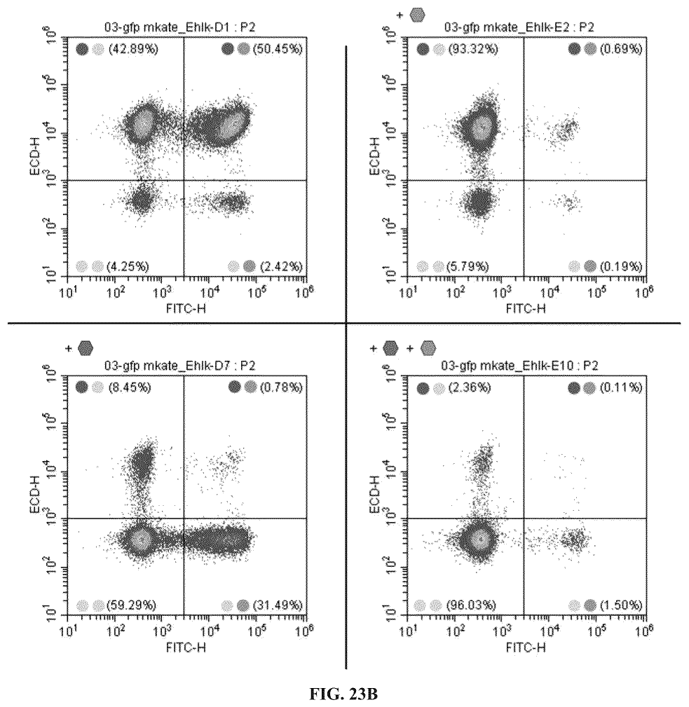

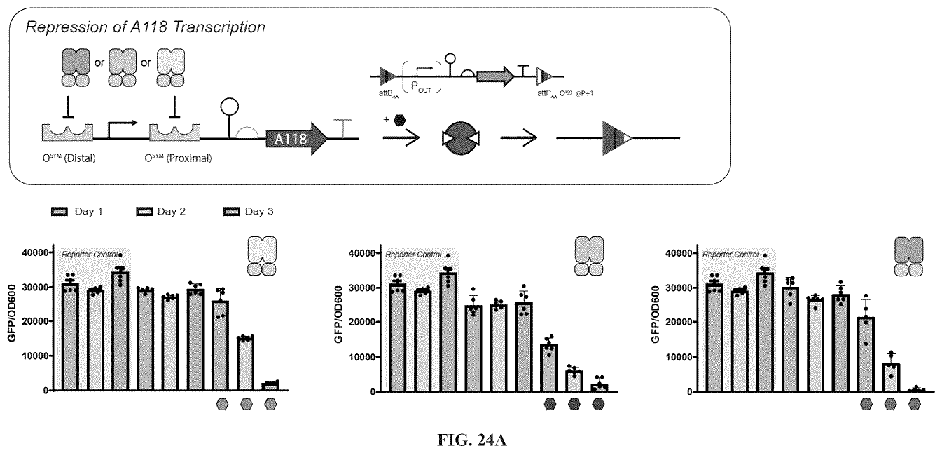

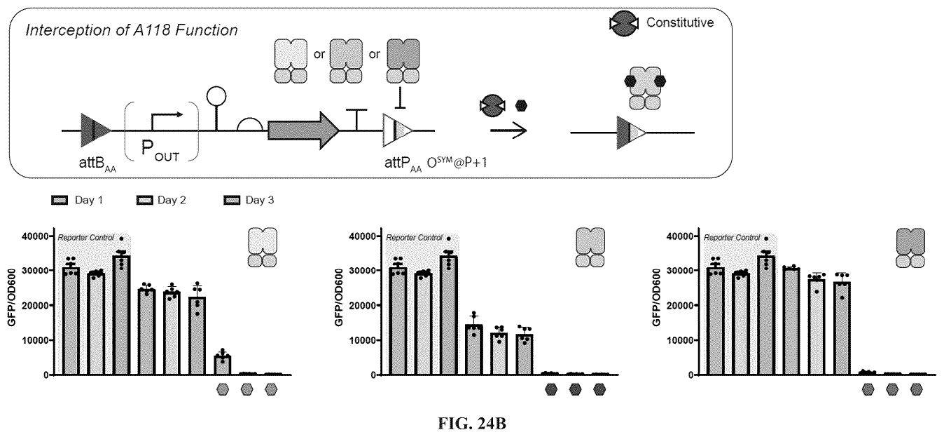

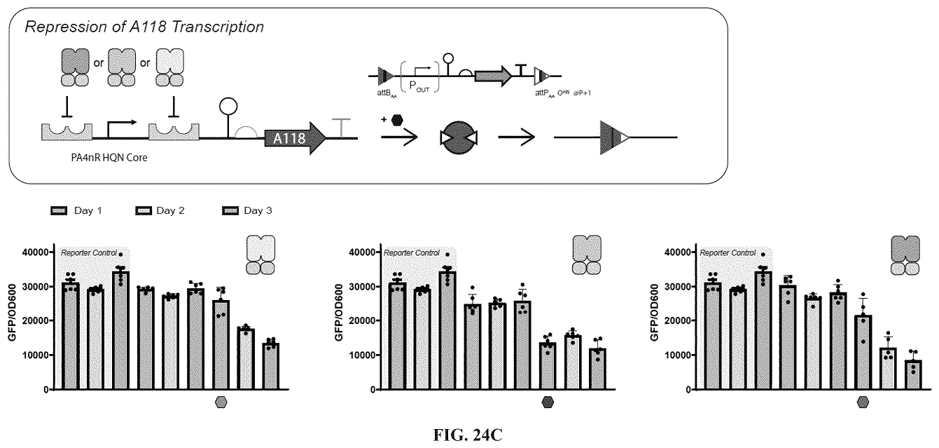

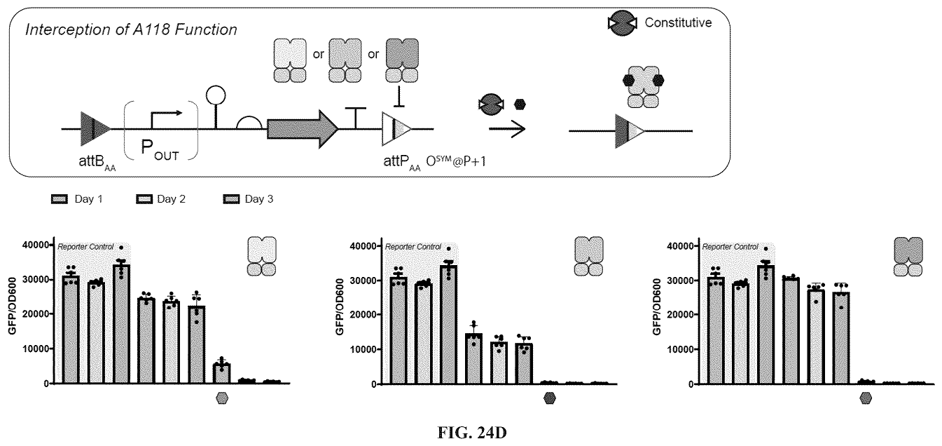

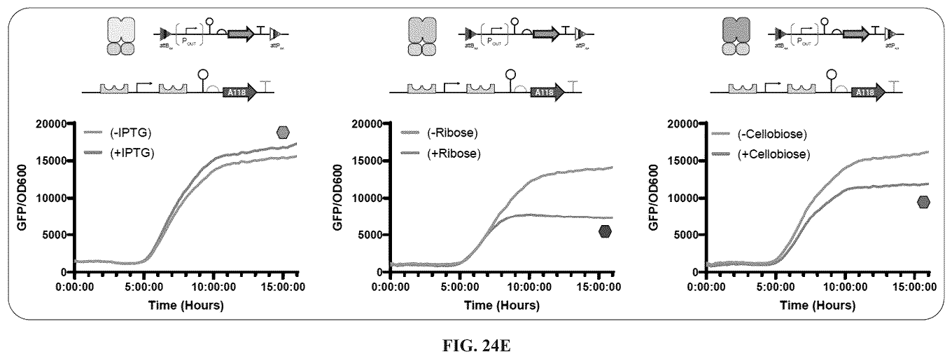

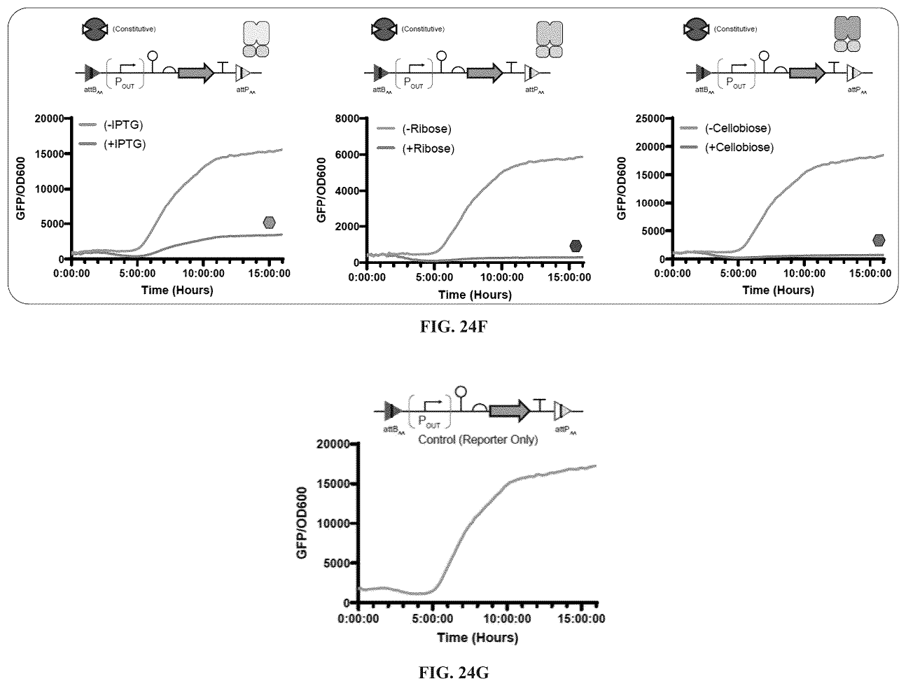

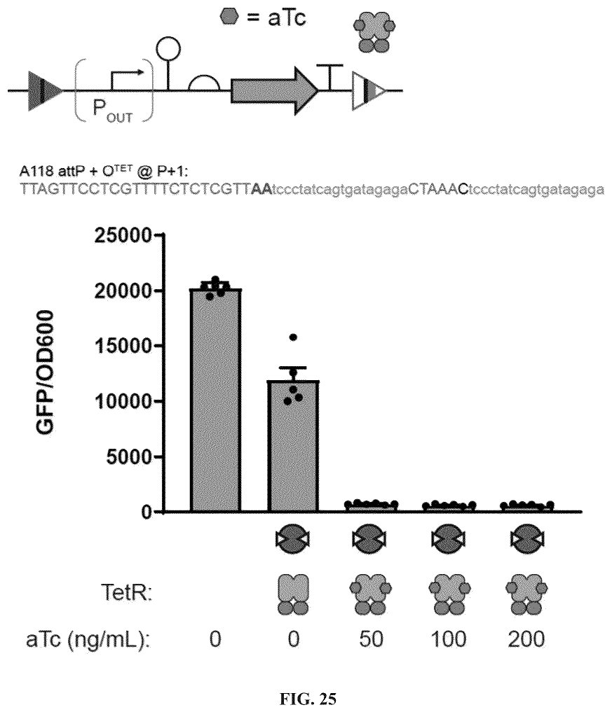

TABLE 1
Sequences in FIGS. 7A-7H.

| Sequence | SEQ ID NO |
|---|---|
| AACTTTTCGGATCAAGCTATGAAAAACGCAAAGAGGGAACTAAACACTT | SEQ ID NO: 5 |
| TTAGTTCCTCGTTTTCTCTCGTTAAAAGAAGAAGAAACGAGAAACTAAA | SEQ ID NO: 6 |
| CGACGTCCAACATATGGCACCGG | SEQ ID NO: 9 |
| GGAGGCTTTCGTCTTTTGACAGCT | SEQ ID NO: 10 |
| CTGAGTAGGACAAATCCGCCGCC | SEQ ID NO: 11 |
| CTAGACCTAGGGTACGGGTTTTGC | SEQ ID NO: 12 |
| ATGCCAACACAATTAACATCTCAATCAAGGTAAATGCTTTTTGCTTTTTT | SEQ ID NO: 13 |
| TGC | |
| TGGCAATTCCGACGTCCAAC | SEQ ID NO: 14 |
| GGAGGCTTTCGTCTTTTGACAGCTAGCTCAG | SEQ ID NO: 15 |
| AGCGAGTTTTTATTTCGTTTATTTCAATTAAGGTAACTAAAAAACTCCTT | SEQ ID NO: 16 |
| T | |
| ACGCTCTCCTGAGTAGGACAAAT | SEQ ID NO: 17 |
| CTAGACCTAGGGTACGGGTTTTGCTG | SEQ ID NO: 18 |
| GTTTGTAAAGGAGACTGATAATGGCATGTACAACTATACTCGTCGGTAA | SEQ ID NO: 19 |
| AAAGGCATCTTAT | |
| AATTCCGACGTCCAACATATGGCACCGGGTT | SEQ ID NO: 20 |
| TTAACGGAGGCTTTCTCTTTTG | SEQ ID NO: 21 |
| ATGGATAAAAAAATACAGCGTTTTTCATGTACAACTATACTAGTTGTAG | SEQ ID NO: 22 |
| TGCCTAAATAATGCTT | |
| CTCTCCTGAGTAGGACAAATCCGCCGCCTTTT | SEQ ID NO: 23 |
| TAACTAGACCTAGGGTACGGGTTT | SEQ ID NO: 24 |
| GAGCGCCGGATCAGGGAGTGGACGGCCTGGGAGCGCTACACGCTGTGG | SEQ ID NO: 25 |
| CTGCGGTCGGTGC | |
| TGCAGGGCTTCCCAACCTTACCAGA | SEQ ID NO: 26 |
| TTGGAGGCTTTCGTCTTTTGACAGCT | SEQ ID NO: 27 |
| CCCTAATACGCAAGTCGATAACTCTCCTGGGAGCGTTGACAACTTGCGC | SEQ ID NO: 28 |
| ACCCTGATCTG | |
| TCCTGAGTAGGACAAATCCGCCGCC | SEQ ID NO: 29 |
| CTAGACCTAGGGTACGGGTTTTGCT | SEQ ID NO: 30 |
| CAATCATCAGATAACTATGGCGGCACGTGCATTAACCACGGTTGTATCC | SEQ ID NO: 31 |
| CGTCTAAAGTACTCGT | |
| GCAATTCCGACGTCCAACATATGGCACCGGGTT | SEQ ID NO: 32 |
| TTAACGGAGGCTTTCGTCTTTTGACAGCTA | SEQ ID NO: 33 |
| TTAATAAACTATGGAAGTATGTACAGTCTTGCAATGTTGAGTGAACAAA | SEQ ID NO: 34 |
| CTTCCATAATAAAAT | |
| TAACTAGACCTAGGGTACGGGTTTTGCTGC | SEQ ID NO: 35 |
| GTTCGTGGTAACTATGGGTGGTACAGGTGCCACATTAGTTGTACCATTT | SEQ ID NO: 36 |
| ATGTTTATGTGGTTAAC | |
| GGCAATTCCGACGTCCAACATATGGCACCGG | SEQ ID NO: 37 |
| GGAGGCTTTCGTCTTTTGACAGCTAGCTCAGTC | SEQ ID NO: 38 |
| TTTTTGTATGTTAGTTGTGTCACTGGGTAGACCTAAATAGTGACACAACT | SEQ ID NO: 39 |
| GCTATTAAAATTTAA | |
| CGCTCTCCTGAGTAGGACAAATCCGCCGCC | SEQ ID NO: 40 |
| CTAGACCTAGGGTACGGGTTTTGCTGCCCGCAA | SEQ ID NO: 41 |
| CGGCCGGCTTGTCGACGACGGCGGTCTCCGTCGTCAGGATCATCCGGGC | SEQ ID NO: 42 |
| GGCTTCCCAACCTTACCAGAGGG | SEQ ID NO: 43 |
| GTCGTGGTTTGTCTGGTCAACCACCGCGGTCTCAGTGGTGTACGGTACA | SEQ ID NO: 44 |
| AACCCCGAC | |
| CTCTCCTGAGTAGGACAAATCCGCCGCC | SEQ ID NO: 45 |
| CTGTTCTGGTGTTGCTAGTTTGTTATCA | SEQ ID NO: 46 |
| GGACGGCGCAGAAGGGGAGTAGCTCTTCGCCGGACCGTCGACATACTG | SEQ ID NO: 47 |
| CTCAGCTCGTC | |
| CGACGTCCAACATATGGCACCGGGTT | SEQ ID NO: 48 |
| TTAACGGAGGCTTTCGTCTTTTGACAGC | SEQ ID NO: 49 |
| GCTCATGTATGTGTCTACGCGAGATTCTCGCCCGAGAACTTCTGCAAGG | SEQ ID NO: 50 |
| CACTGCTCTTGGCT | |
| CTGAGTAGGACAAATCCGCCGCCTTTT | SEQ ID NO: 51 |
| TAACTAGACCTAGGGTACGGGTTTTGCTGCC | SEQ ID NO: 52 |

FIGS. 8A-8L depict recombinase attachment half-site omission and design heuristics for engineering an interception synthetic memory circuit. FIG. 8A shows a schematic of the recombinase deletion circuit, in which a reporter circuit comprised of a constitutive promoter, ribozyme, RBS, and green fluorescent protein (GFP) reading frame flanked by an aligned attB and attP pair (STATE 1). Note: A genetic insulator (ribozyme) was used to catalyze the removal of the 5′ UTR of the transcript to normalize GFP expression. A recombinase (RECOM) matched to the given att sites catalyzes recombination between attB and attP, resulting in the deletion of the entire circuit (STATE 2). FIGS. 8B-8I show half-site omission tests with various recombinases are shown. For each plot, the two data bars shown in the shaded area represent controls; S1 measures the fluorescence of cells transformed with the reporter plasmid alone, and S2 measures the fluorescence of cells transformed with the reporter plasmid plus the corresponding constitutive recombinase expression plasmid. In the unshaded areas, half-site omissions are shown with the cognate recombinases present. B1 refers to a construct in which the first half of the attB site has been omitted, B2 refers to a construct where the second half of the attB site has been omitted, likewise, half-site omissions P1 and P2 correspond to positions in the attP site. Details for each recombinase are given in FIGS. 7A-7H. FIG. 8J shows a granular description of an attachment site substituted with a 16-base pair operator. In this example, the O^{ttg} operator is substituted within an attP site that corresponds to recombinase A118. The Ong DNA operator is cognate to the E⁺_{HON} transcription factor. FIG. 8K shows a schematic of the putative mechanism of interception; in the grey box at left, repressor binding at the O^{ttg} DNA operator (within the attP site) protects the circuit from deletion catalyzed by the A118 recombinase. To the right, when the repressor is induced, it unbinds from the att site, and A118 can recombine and delete the circuit. FIG. 8L shows a variety of operator positions were tested, identifying the P+1 location as the best candidate for controlling recombination with interception. Data in FIGS. 8B-8I and FIG. 8L represent the average of n=6 biological replicates. Error bars correspond to the SEM of these measurements. Sequences in FIGS. 8A-8L are provided in TABLE 2.

TABLE 2
Sequences in FIGS. 8A-8L.

| Sequence | SEQ ID NO |
|---|---|
| TTTTGAGCGCTCAAAA | SEQ ID NO: 53 |
| TTAGTTCCTCGTTTTCTCTCGTTAATTTTGAGCGCTCAAAAAAACTAAA | SEQ ID NO: 54 |
| TTTTGAGCGCTCAAAATCTCGTTAAAAGAAGAAGAAACGAGAAACTAAA | SEQ ID NO: 55 |
| TTAGTTTTTTGAGCGCTCAAAATAAAAGAAGAAGAAACGAGAAACTAAA | SEQ ID NO: 56 |
| TTAGTTCCTTTTTGAGCGCTCAAAAAAGAAGAAGAAACGAGAAACTAAA | SEQ ID NO: 57 |
| TTAGTTCCTCTTTTGAGCGCTCAAAAAGAAGAAGAAACGAGAAACTAAA | SEQ ID NO: 58 |
| TTAGTTCCTCGTTTTCTCTCGTTAAAATTTTGAGCGCTCAAAAACTAAA | SEQ ID NO: 59 |
| TTAGTTCCTCGTTTTCTCTCGTTAAAAGTTTTGAGCGCTCAAAACTAAA | SEQ ID NO: 60 |

FIGS. 9A-9H depict relevant plasmid maps used herein.

FIGS. 10A-10F depict interception synthetic memory with expanded information processing and operator variation. FIG. 10A shows a schematic summarizing the mechanism and genetic construct (deletion circuit) used to assess A118 recombinase interception with variable repressors directed at different operators placed in the P+1 position (described in FIG. 8J). The A118 recombinase and relevant repressor are constitutively expressed in all cases. The repressors used are comprised of two modular domains: (i) a regulatory core domain that allows the repressor to be induced by a different ligand, and (ii) a DNA-binding domain that allows the repressor to bind to a different operator. In STATE 1, repressor binding at the operator blocks recombinase function, protecting the circuit from deletion. Inducing the repressor brings the circuit to STATE 2, where the recombinase can access the attP site to recombine the circuit (bringing the circuit to STATE 3). FIG. 10B shows assay data for intercepted (minus ligand) circuits versus deprotected (induced) circuits using the repressor E⁺ across six different DNA-binding domain/operator pairs. FIGS. 10C-10F shows assay data using the same set of DNA-binding domain/operator pairs as b paired with different regulatory core domains as follows: FIG. 10C shows R⁺, FIG. 10D shows F⁺, FIG. 10E shows G⁺, and FIG. 10F shows I⁺. (inset) Symbols for the circuit parts and modular repressor components used, including six DNA-binding domains conferring alternate DNA recognition, the six DNA operators where those DNA-binding domains can bind (color-matched), and the five regulatory core domains sensitive to different ligands. Data in FIGS. 10B-10F represent the average of n=6 biological replicates. Error bars correspond to the SEM of these measurements.

FIGS. 11A-11H depict transcriptional repressor performance compared to repressor interception. In FIG. 11A at top left, the performance card for a general repressor (X+) and an abstraction of its performance metrics to a logical BUFFER operation is shown. At top right, the performance card of a general anti-repressor (X^{A}) and an abstraction of its performance metrics to a logical NOT operation is shown. The metrology for a given single-INPUT single-OUTPUT (SISO) operation, the induction profile can be modeled for an experimentally verified SISO BUFFER operation via a coarse-grained binding function defined as

$${Ω}^{+} ( I ) = σ {Λ}^{+} ( I ) + ε \qquad ( Eq . 1 )$$

where σ is a constant representing the maximum fluorescence-relative to basal expression of the OFF-state, Λ⁺ (I) is a coarse-grained Hill function that can assume a value of 0 or 1, and ε represents fluorescence in the absence of inducer—i.e., the OFF-state. To model the performance of a given SISO NOT gate an analogous coarse-grained binding function was used-though for anti-repression—defined as

$${Ω}^{A} ( I ) = σ {Λ}^{A} ( I ) + ε \qquad ( Eq . 2 )$$

where σ is a constant representing the maximum fluorescence, minus ligand-relative to basal expression of the OFF-state, Λ^{A}(I) is a coarse-grained antithetical Hill-function for anti-repression where 0 INPUT corresponds to the ON-state, and 1 INPUT corresponds to the OFF-state, and ε represents fluorescence in the presents of inducer—i.e., the OFF-state. This collection of models were used to study performance prediction in Milner et al.2 At bottom left, the front of a general performance card is shown detailing the plasmids, PROXIMAL operator position, and chassis used to measure the TF performance data. At bottom right, the wildtype A118 attP site shown in blue (with central conserved region shown in grey) is compared to the attP sites used for interception with operators substituted at position P+1 (see FIG. 8J). Nucleotides that are altered by the inclusion of the given operator are shown in red, highlighting the alteration to the attP site incurred by substituting each operator. The sequence similarity between each operator substituted attP site and wildtype is given as Match %, with lower scores indicating lower similarity.

FIG. 11B compares interception performance of E⁺ variants to the transcriptional repression performance of those same E⁺ variants. At the top left, assay data is shown for intercepted (minus ligand) circuits versus deprotected (induced) circuits using the repressor E⁺ across six different DNA-binding domain/operator pairs. At the top right, the performance card for a general repressor (X⁺) and an abstraction of its performance metrics to a logical BUFFER operation is shown. Below, transcriptional repression performance cards for each of the E⁺ variants tested for interception performance are given.

FIGS. 11C-11F show a similar comparison as given in FIG. 11B between repressor interception performance and transcriptional repression performance is provided for RCDs R⁺, F⁺, G⁺, and I⁺. Data in FIGS. 11B-11F represent the average of n=6 biological replicates. The detail sequences for each substitution is given in FIG. 11G. Error bars correspond to the SEM of these measurements.

FIG. 11Ha-11Hj shows flow cytometry of select interception circuits. FIG. 11Ha shows an inset at the left which is a schematic summarizing the genetic construct used to assess A118 recombinase interception with variable repressors directed at different operators placed in the P+1 position. Inset at right is the subset of modular TF components used for flow cytometry analysis of interception performance. FIGS. 11Hb-11Hj show flow cytometry analysis of different TFs intercepting A118 via different operators in the P+1 position. The distribution of cells in the two possible states of fluorescent protein expression after growth in a medium without inducers is shown. Data in FIGS. 11Hb-11Hj represent the average of n=6 biological replicates. Error bars correspond to the SEM of these measurements. Sequences in FIGS. 11A-11H are provided in TABLE 3.

TABLE 3
Sequences in FIGS. 11A-11H.

| Sequence | SEQ ID NO |
|---|---|
| TTAGTTCCTCGTTTTCTCTCGTTAAAAGAAGAAGAAACGAGAAACTAAA | SEQ ID NO: 6 |
| AGCGAGTTTTTATTTCGTTTATTTCAATTAAGGTAACTAAAAAACTCCTT | SEQ ID NO: 16 |
| T | |
| GTTTGTAAAGGAGACTGATAATGGCATGTACAACTATACTCGTCGGTAA | SEQ ID NO: 19 |
| AAAGGCATCTTAT | |
| ATGGATAAAAAAATACAGCGTTTTTCATGTACAACTATACTAGTTGTAG | SEQ ID NO: 22 |
| TGCCTAAATAATGCTT | |
| CCCTAATACGCAAGTCGATAACTCTCCTGGGAGCGTTGACAACTTGCGC | SEQ ID NO: 28 |
| ACCCTGATCTG | |
| TTAATAAACTATGGAAGTATGTACAGTCTTGCAATGTTGAGTGAACAAA | SEQ ID NO: 34 |
| CTTCCATAATAAAAT | |
| TTTTTGTATGTTAGTTGTGTCACTGGGTAGACCTAAATAGTGACACAACT | SEQ ID NO: 39 |
| GCTATTAAAATTTAA | |
| GTCGTGGTTTGTCTGGTCAACCACCGCGGTCTCAGTGGTGTACGGTACA | SEQ ID NO: 44 |
| AACCCCGAC | |
| GCTCATGTATGTGTCTACGCGAGATTCTCGCCCGAGAACTTCTGCAAGG | SEQ ID NO: 50 |
| CACTGCTCTTGGCT | |
| TTAGTTCCTCGTTTTCTCTCGTTAATTTTGAGCGCTCAAAAAAACTAAA | SEQ ID NO: 54 |
| ATTXYZAGCGGATAACAAT | SEQ ID NO: 61 |
| AEYAGVSADTVSRVVNQASHV | SEQ ID NO: 62 |
| AATTXYZAGCGGATAACAATT | SEQ ID NO: 63 |
| AAAGAGGAGAAA | SEQ ID NO: 64 |
| TTAGTTCCTCGTTTTCTCTCGTTAATTCTTAGCGCTAAGAAAAACTAAA | SEQ ID NO: 65 |
| TTAGTTCCTCGTTTTCTCTCGTTAATTGACAGCGCTGTCAAAAACTAAA | SEQ ID NO: 66 |
| TTAGTTCCTCGTTTTCTCTCGTTAATTAGGAGCGCTCCTAAAAACTAAA | SEQ ID NO: 67 |
| TTAGTTCCTCGTTTTCTCTCGTTAATTTTAAGCGCTTAAAAAAACTAAA | SEQ ID NO: 68 |
| TTAGTTCCTCGTTTTCTCTCGTTAATTGTGAGCGCTCACAAAAACTAAA | SEQ ID NO: 69 |
| ATTGTGAGCGCTCACAATT | SEQ ID NO: 70 |
| AEYAGVSYQTVSRVVNQASHV | SEQ ID NO: 71 |
| ATTGACAGCGCTGTCAATT | SEQ ID NO: 72 |
| AEYAGVSGKTVSRVVNQASHV | SEQ ID NO: 73 |
| ATTTTAAGCGCTTAAAATT | SEQ ID NO: 74 |
| AEYAGVSTATVSNVVNQASHV | SEQ ID NO: 75 |
| ATTTTGAGCGCTCAAAATT | SEQ ID NO: 76 |
| AEYAGVSHQTVSNVVNQASHV | SEQ ID NO: 77 |
| ATTCTTAGCGCTAAGAATT | SEQ ID NO: 78 |
| AEYAGVSHTTVSKVVNQASHV | SEQ ID NO: 79 |
| ATTAGGAGCGCTCCTAATT | SEQ ID NO: 80 |
| AEYAGVSKSTVSLVVNQASHV | SEQ ID NO: 81 |
| GCTCATGTATGTGTCTACGCGAGATTCTCGCCTTGTGAGCGCTCACAAG | SEQ ID NO: 82 |
| CACTGCTCTTGGCT | |
| CCCTAATACGCAAGTCGATAACTCTCCTGGGAGCGTTTTGAGCGCTCAA | SEQ ID NO: 83 |
| AACCTGATCTG | |
| TTTTTGTATGTTAGTTGTGTCACTGGGTAGACTTGTGAGCGCTCACAACT | SEQ ID NO: 84 |
| GCTATTAAAATTTAA | |
| GTCGTGGTTTGTCTGGTCAACCACCGCGGTTTTTGAGCGCTCAAAAACA | SEQ ID NO: 85 |
| AACCCCGAC | |
| AGCGAGTTTTTATTTCGTTTATTTCTTTTGAGCGCTCAAAAAAACTCCTT | SEQ ID NO: 86 |
| T | |
| GTTTGTAAAGGAGACTGATAATGGCATGTACAACTATACTTTGTGAGCG | SEQ ID NO: 87 |
| CTCACAATCTTAT | |
| ATGGATAAAAAAATACAGCGTTTTTCATGTACAACTATACTTTGTGAGC | SEQ ID NO: 88 |
| GCTCACAATAATGCTT | |
| TTAATAAACTATGGAAGTATGTACAGTCTTGCAATTGTGAGCGCTCACA | SEQ ID NO: 89 |
| ATTCCATAATAAAAT | |
| TTAATAAACTATGGAATTGTGAGCGCTCACAAAATGTTGAGTGAACAAA | SEQ ID NO: 90 |
| CTTCCATAATAAAAT | |

FIG. 12 depicts the ribosome binding site (RBS) tuning of A118 for three R⁺ ADRs. (top) A schematic summarizing the mechanism and genetic construct (deletion circuit) used to assess A118 recombinase interception with variable repressors directed at different operators placed in the P+1 position. (bottom left) Assay data collected under moderate conditions for R⁺, also given in FIGS. 10A-10F. (bottom right) Assay data for three ADR/operator pairs, GKR/O^{gac}, TAN/O^{tta}, and HTK/O^{ctt} following A118 expression modification via a prescribed RBS library. Data represents the average of n=6 biological replicates. Error bars correspond to the SEM of these measurements.

FIGS. 13A-13F depict permissive interception synthetic memory via anti-repression. FIG. 13A shows a schematic summarizing the mechanism and genetic construct (deletion circuit) used to assess A118 recombinase interception for different antirepressors directed at different operators placed in the P+1 position (described in FIG. 8J). Anti-repressors have the opposite induction/DNA binding relationship to repressors; anti-repressors bind DNA when induced and do not bind DNA when not induced. The A118 recombinase and relevant anti-repressor are constitutively expressed in all cases. As in FIGS. 10A-10F, the anti-repressor used are comprised of two modular domains: (1) a regulatory core domain that allows the anti-repressor to be induced by a different ligand, and (2) a DNA-binding domain that allows the anti-repressor to bind to a different operator. In STATE 1, induced anti-repressor binding at the operator blocks the recombinase function, protecting the circuit from deletion. In the absence of ligand, the anti-repressor cannot bind to the operator, bringing the circuit to STATE 2, where the recombinase can access the attP site to recombine the circuit (bringing the circuit to STATE 3). FIG. 13B shows assay data for deprotected (minus ligand) circuits versus intercepted (induced) circuits using I^{A(5)} across six different DNA-binding domain/operator pairs. FIGS. 13C-13F show assay data using the same set of DNA-binding domain/operator pairs as FIG. 13B paired with different regulatory core domains as follows: FIG. 13C shows R^{A(I)}, FIG. 13D shows F^{A(I)} FIG. 13E shows S^{A(I)}, and FIG. 13F shows P^{A}. (inset) Symbols for the circuit parts and modular anti-repressor components used, including 6 DNA-binding domains conferring alternate DNA recognition, the six DNA operators where those DNA-binding domains can bind (color-matched), and five regulatory core domains sensitive to different ligands. Data in FIGS. 13B-13F represent the average of n=6 biological replicates. Error bars correspond to the SEM of these measurements.

FIGS. 14A-14E depict transcriptional repressor performance compared to anti-repressor interception. FIG. 14A compares interception performance of I^{A(5)} variants to transcriptional anti-repression performance of those same I^{A(5)} variants. At top left, assay data is shown for intercepted (induced) circuits versus deprotected (minus ligand) circuits using the anti-repressor I^{A(5)} across six different DNA-binding domain/operator pairs. At top right, the performance card for a general anti-repressor (X^{A}) and an abstraction of its performance metrics to a logical NOT operation is shown. Below, transcriptional anti-repression performance cards for each of the I^{A(5)} variants tested for interception performance are given. FIGS. 14B-14E show a similar comparison as given in FIG. 14A between anti-repressor interception performance and transcriptional anti-repression performance is provided for RCDs R^{A(1)}, F^{A(1)}, S^{A(1)}, and P^{A}. Data in FIGS. 14A-14E represent the average of n=6 biological replicates. Error bars correspond to the SEM of these measurements. Sequences in FIGS. 14A-14E are provided in TABLE 4.

TABLE 4
Sequences in FIGS. 14A-14E.

| Sequence | SEQ ID NO |
|---|---|
| AEYAGVSADTVSRVVNQASHV | SEQ ID NO: 62 |
| AATTXYZAGCGGATAACAATT | SEQ ID NO: 63 |
| ATTGTGAGCGCTCACAATT | SEQ ID NO: 70 |
| AEYAGVSYQTVSRVVNQASHV | SEQ ID NO: 71 |
| ATTGACAGCGCTGTCAATT | SEQ ID NO: 72 |
| AEYAGVSGKTVSRVVNQASHV | SEQ ID NO: 73 |
| ATTTTAAGCGCTTAAAATT | SEQ ID NO: 74 |
| AEYAGVSTATVSNVVNQASHV | SEQ ID NO: 75 |
| ATTTTGAGCGCTCAAAATT | SEQ ID NO: 76 |
| AEYAGVSHQTVSNVVNQASHV | SEQ ID NO: 77 |
| ATTCTTAGCGCTAAGAATT | SEQ ID NO: 78 |
| AEYAGVSHTTVSKVVNQASHV | SEQ ID NO: 79 |
| ATTAGGAGCGCTCCTAATT | SEQ ID NO: 80 |
| AEYAGVSKSTVSLVVNQASHV | SEQ ID NO: 81 |
| AEYAGVSFKTVSRVVNQASHV | SEQ ID NO: 91 |

FIG. 15 depicts the ribosome binding site (RBS) tuning of A118 for three R^{A(1)} ADRs. (top) Schematic summarizing the mechanism and genetic construct (deletion circuit) used to assess A118 recombinase interception for different anti-repressors directed at different operators placed in the P+1 position. (bottom left) Assay data collected under moderate conditions for R^{A(1)}, also given in FIGS. 11A-11H. (bottom right) Assay data for three ADR/operator pairs, GKR/O^{gac}, TAN/O^{tta}, and HTK/O^{ctt} following A118 expression modification via RBS library. Data represents the average of n=6 biological replicates. Error bars correspond to the SEM of these measurements.

FIGS. 16A-16J depict nested BUFFER and NOT logic paired with interception memory. FIG. 16A shows a schematic of a BUFFER logic gate consisting of GFP regulated by R⁺_{YQR} directed to the O^{gtg} operator in the CORE position nested within an A118-mediated deletion circuit, intercepted via E⁺_{HQN} and cognate operator O^{tgg} at position P+1. A118 is constitutively expressed in all assays. When R⁺_{YQR} is induced, GFP OUTPUT is expressed—i.e., the circuit operates as a simple BUFFER gate. When E⁺_{HQN} is induced, the circuit is deprotected and A118 recombines the att sites, deleting the nested BUFFER gate. FIG. 16B shows assay data for the given circuit controlled by R⁺_{YQR} and intercepted by E⁺_{HQN} under different inducer conditions. Inducing only R⁺_{YQR} results in the production of the GFP OUTPUT—i.e., the circuit functions as a BUFFER logic operation. Inducing E⁺_{HQN} enables A118-mediated deletion of the nested logic circuit, observed as mitigated GFP fluorescence. The assay condition with only cellobiose added, boxed in red, was diluted and grown in fresh minimal media for 20 additional hours with and without ribose. The data in the red box shows the resulting phenotypes, confirming deletion. FIG. 16C shows assay data for this circuit controlled by an alternate TF set—i.e., BUFFER logic controlled by E⁺_{YQR} and interception by I⁺_{HQN}. Inducing only E⁺_{YQR} results in GFP OUTPUT—i.e., functioning as a simple BUFFER logical operation. Inducing I⁺_{HQN} enables A118-mediated deletion of the nested logic circuit, resulting in mitigated GFP fluorescence. The assay condition with only IPTG added, boxed in red, was diluted and grown in fresh minimal media for 20 additional hours with and without cellobiose. The data in the red box shows the resulting phenotypes. FIGS. 16D-16F are additional nested logical operations via alternate transcription factors, i.e., with the BUFFER operator O^{gtg} in the PROXIMAL position. FIG. 16G shows a schematic of a NOT logic gate consisting of GFP regulated by I^{A(9)}_{YQR} directed to the O^{gtg} operator in the CORE position nested within an A118 deletion circuit that is intercepted by E⁺_{HQN} at the O^{ttg} operator placed at the P+1 position. FIG. 16H shows assay data for the given circuit controlled by I^{A(9)}_{YQR} and intercepted by E⁺_{HQN}. Inducing only I^{A(9)}_{YQR} resulted in mitigated GFP OUTPUT—i.e., the circuit objectively functions as a NOT logical operation. Inducing E⁺_{HQN} enables A118-mediated deletion of the nested circuit, resulting in abrogated NOT logic. The assay condition with only cellobiose added, boxed in red, was diluted and grown in fresh minimal media for 20 additional hours with and without IPTG. The data in the red box shows the resulting phenotypes, confirming circuit deletion. FIGS. 161-16J are similar to FIGS. 16G-16H, however with the NOT operator O^{gtg} in the PROXIMAL position. Data in FIGS. 16A-16H represent the average of n=6 biological replicates. Error bars correspond to the SEM of these measurements.

FIGS. 17A-17D depict interception synthetic memory with nested AND/NOR Boolean Logic. Interception memory circuits were constructed with nested 2-INPUT logic. Namely, a circuit was constructed with a nested AND gate (see FIGS. 17A-17B) and a separate circuit with a nested NOR gate (see FIGS. 17C-17D). In the first iteration of the 2-INPUT circuit, the nested AND logic with interception was functional, requiring 2-INPUTs to produce GFP—unless the attachment site was deprotected upon the addition of cellobiose see FIGS. 17A-17B. Moreover, in the absence of recombinase, the circuit performed as a simple AND gate—independent of the presence of cellobiose (see FIG. 17B inset blue box). Likewise, the nested NOR iteration of the interception circuit was also functional—with a synonymous control feature regulating the deletion memory operation (see FIGS. 17C-17D). Namely, the protected circuit only produced GFP in the absence of both IPTG and ribose. However, upon the addition of cellobiose, the circuit was deleted (see FIGS. 17C-17D). FIG. 17A shows a genetic schematic and mechanism of a transcriptional AND gate nested within a memory interception circuit (coded for deletion), cognate to the A118 recombinase. A118 and the regulating TFs (I⁺_{YQR}, R⁺_{YQR}, and E⁺_{HQN}) are expressed constitutively. I⁺_{YQR} and R⁺_{YQR} regulate GFP expression by binding to an O^{gtg} operator in the promoter's core position, and E⁺_{HQN} intercepts the A118 function at an Ottg promoter placed at the P+1 position. From left to right, the response to different inducers (INPUTs) is shown. FIG. 17B shows assay data for the circuit shown in FIG. 17A. Cells exposed to cellobiose (i.e., post deletion) are boxed in red. Post deletion, cells were diluted 1:200 and grown in fresh minimal media for 20 additional hours with and without the transcriptional logic INPUTS IPTG and ribose to demonstrate deletion memory. Data in the red box labeled DAY 2 shows the resulting phenotypes. To the right (of the red box), boxed in blue, is the control data for this circuit transformed with the TF-expression plasmid and without the recombinase expression plasmid. FIG. 17C shows a genetic schematic of a transcriptional NOR gate nested within a memory interception circuit (coded for deletion), cognate to the A118 recombinase. A118 and the regulating TFs (I^{A(9)}_{YQR}, R^{A}(2)_{YQR}, and E⁺_{HQN}) are expressed constitutively. I^{A(9)}_{YQR} and R^{A}(2)_{YQR} regulate GFP expression by binding to an O^{gtg} operator in the promoter's core position, and E⁺_{HQN} intercepts A118 function at an O^{ttg} promoter placed at the P+1 position. FIG. 17D shows assay data for the circuit shown in FIG. 17C. Cells exposed to cellobiose (i.e., post deletion) are boxed in red. Post deletion, cells were diluted 1:200 and grown in fresh minimal media for 20 additional hours with and without the transcriptional logic INPUTS IPTG and ribose to demonstrate deletion memory. Data in the red box shows the resulting phenotypes. To the right (of the red box), boxed in blue, is the control data for this circuit transformed with the TF-expression plasmid and without the recombinase expression plasmid. Data in FIG. 17B and FIG. 17D represent the average of n=6 biological replicates. Error bars correspond to the SEM of these measurements.

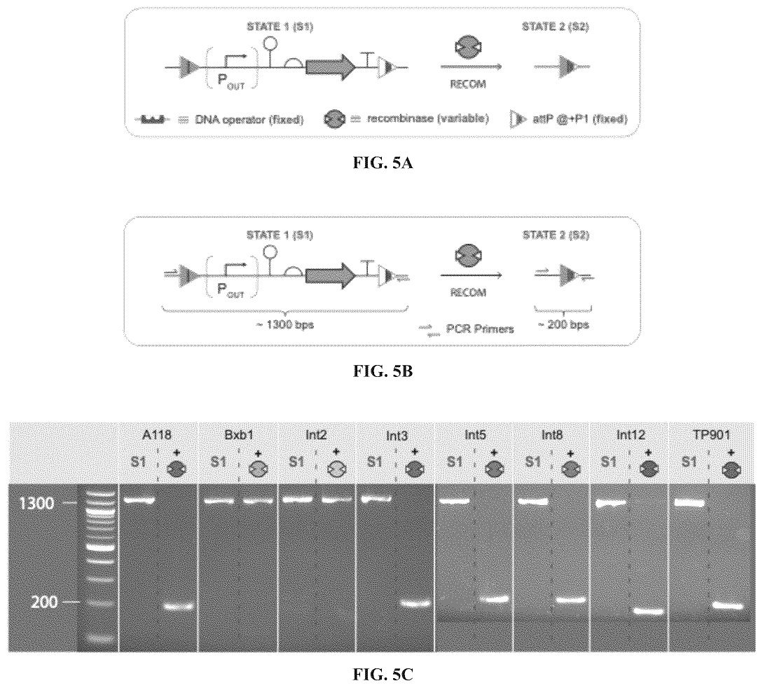

FIGS. 18A-18I depict interception via orthogonal recombinase functions. FIG. 18A shows a schematic summarizing the mechanism and genetic construct (deletion circuit) used to assess interception for eight different recombinases regulated by E⁺_{HQN} directed to the cognate operator substituted at the P+1 position for each recombinase (also see FIGS. 7A-7H and FIGS. 8A-8L). The relevant recombinase and E⁺_{HQN} are constitutively expressed in all cases. As in FIGS. 10A-10F, in STATE 1, E⁺_{HQN} binding at the operator blocks recombinase function, protecting the circuit from deletion. Inducing the repressor brings the circuit to STATE 2, where the recombinase can access the attP site to recombine the circuit (bringing the circuit to STATE 3). FIG. 18B shows data in the grey box which represents control data; the left bar displays data for E. coli cells containing the reporter (GFP) plasmid alone, representing maximum fluorescence. The right bar displays data for E. coli cells containing the reporter and recombinase expression plasmids. However, the E⁺_{HQN} repressor is not present; accordingly, interception is not possible; thus, the circuit is deprotected. Data in the red box displays the effect of interception on the circuit for E. coli containing all three plasmids (reporter, recombinase, and repressor); on the left is the intercepted (minus ligand) circuit, and on the right is the deprotected (induced) circuit. FIGS. 18C-18I show assay data following the same format as FIG. 18B for different recombinases as follows: FIG. 18C shows TP901, FIG. 18D shows Int12, FIG. 18E shows Int8, FIG. 18F shows Int3, FIG. 18G shows Int5, FIG. 18H shows Bxb1, and FIG. 18I shows Int2—also see FIGS. 5A-5C for qualitative genotype data. Inset at the bottom is a schematic defining the colors for different recombinases and their cognate attachment sites. Data in FIGS. 18B-18I represent the average of n=6 biological replicates. Error bars correspond to the SEM of these measurements.

FIGS. 19A-19F depict additional operator positions, interception via A118, TP901, Int3, Int5, and Int12. FIG. 19A shows a schematic summarizing the genetic construct used to assess the interception of five different recombinases by E⁺_{HQN} directed at O^{ttg} via variable operator positions. The relevant recombinase and E⁺_{HQN} are constitutively expressed in all cases. In STATE 1, E⁺_{HQN} binding at the operator blocks the recombinase function, protecting the circuit from deletion. Inducing the repressor brings the circuit to STATE 2, where the recombinase can access the attP site to recombine the circuit, bringing the circuit to STATE 3. FIG. 19B shows alternate operator positions that facilitate interception via the A118 recombinase. Inset text displays the genetic edits made to include O^{gtg} operators (shown in red and underlined) at those attP positions (shown in blue, with central conserved region shown in black). FIG. 19C shows alternate interception operator positions for the recombinase TP901. FIG. 19D shows alternate operator positions for the recombinase Int3. FIG. 19E shows alternate operator position for the recombinase Int5. FIG. 19F shows alternate operator position for the recombinase Int12. Data in FIGS. 19B-19F represent the average of n=6 biological replicates. Error bars correspond to the SEM of these measurements. Sequences in FIGS. 19A-19F are provided in TABLE 5.

TABLE 5
Sequences in FIGS. 19A-19F.

| Sequence | SEQ ID NO |
|---|---|
| TTAGTTTTTTGAGCGCTCAAAATAAAAGAAGAAGAAACGAGAAACTAA | SEQ ID NO: 56 |
| A | |
| TTAGTTCCTTTTTGAGCGCTCAAAAAAGAAGAAGAAACGAGAAACTAA | SEQ ID NO: 57 |
| A | |
| AGTTTTGAGCGCTCAAAATTATTTCAATTAAGGTAACTAAAAAACTCCT | SEQ ID NO: 92 |
| TT | |
| AGCGAGTTTTTGAGCGCTCAAAATCAATTAAGGTAACTAAAAAACTCC | SEQ ID NO: 93 |
| TTT | |
| AGCGAGTTTTTATTTCGTTTATTTCAATTTTGAGCGCTCAAAAACTCCTT | SEQ ID NO: 94 |
| T | |
| ATGGATAAAAAAATACAGCGTTTTTCATGTACAACTATACTATTTTGAG | SEQ ID NO: 95 |
| CGCTCAAAAAATGCTT | |
| ATGGATAAAAAAATACAGCGTTTTTCATGTACAACTATACTAGTTTTGA | SEQ ID NO: 96 |
| GCGCTCAAAAATGCTT | |
| ATGGATAAAAAAATACAGCGTTTTTCATGTACAACTATACTAGTTGTTT | SEQ ID NO: 97 |
| TGAGCGCTCAAAACTT | |
| ATGGATAAAAAAATACAGCGTTTTTCATGTACAACTATACTAGTTGTTT | SEQ ID NO: 98 |
| TTGAGCGCTCAAAATT | |
| CCCTATTTTGAGCGCTCAAAACTCTCCTGGGAGCGTTGACAACTTGCGC | SEQ ID NO: 99 |
| ACCCTGATCTG | |
| TTTTTGTATGTTAGTTGTGTCACTGGGTAGACCTAAATAGTGTTTTGAG | SEQ ID NO: 100 |
| CGCTCAAAAAATTTAA | |

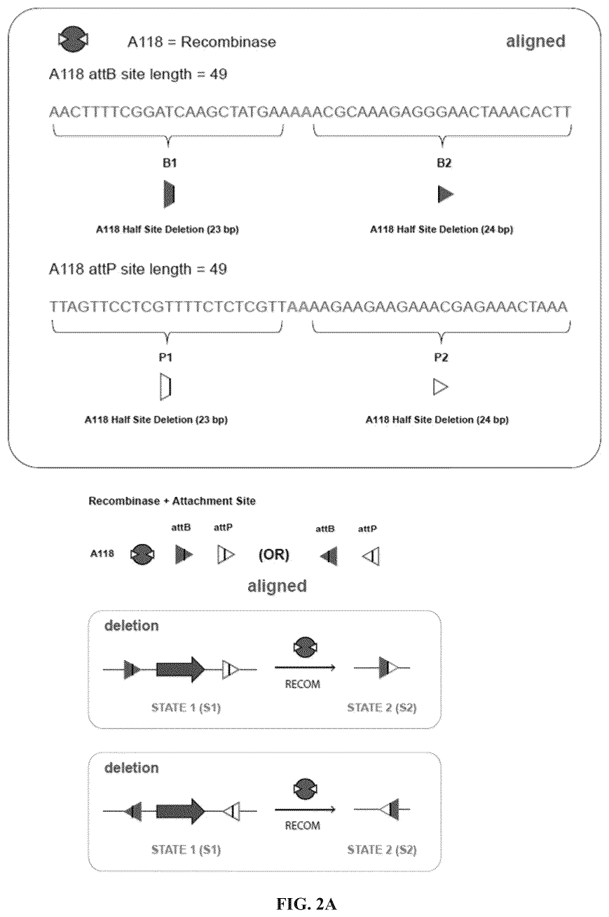

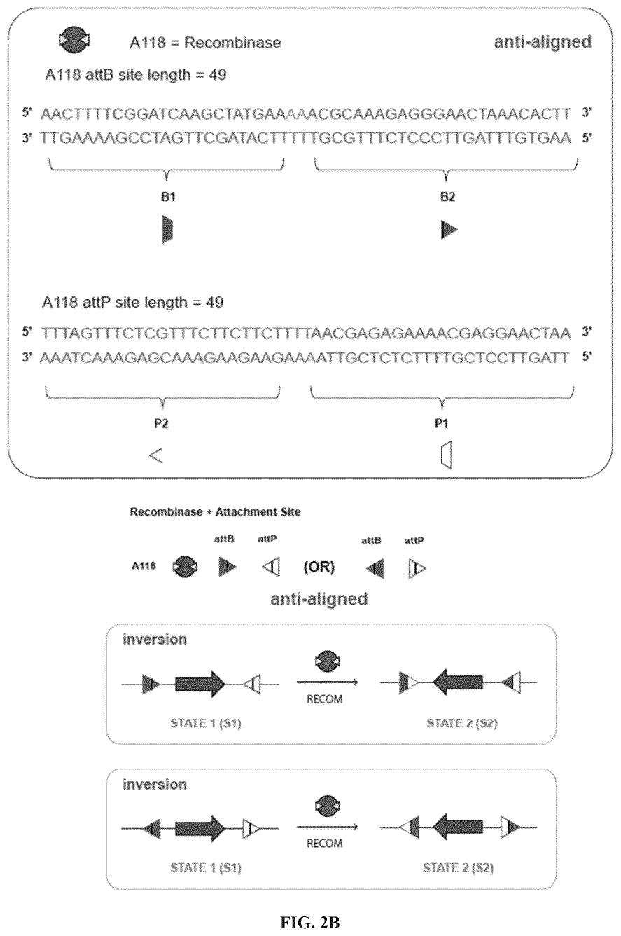

FIGS. 20A-20D depict the interception synthetic memory facilitating controlled DNA inversions. FIGS. 20A-20D show promoter inversions controlled by interception demonstrated for four different recombinases, FIG. 20A shows A118, FIG. 20B shows Int3, FIG. 20C shows Int8, and FIG. 20D shows Int12. For each subfigure FIGS. 20A-20D, diagram (1) at top left shows a schematic of the corresponding promoter inversion circuit. In this state, the promoter is in the opposite orientation to transcribe GFP, corresponding to reduced fluorescence in the fluorescence assay data (shown in the right corner). As shown in diagram (2), adding I⁺_{HQN} repressor protects the circuit from recombinase-mediated inversion via binding to the O^{ttg} operator at position P+1. As shown in diagram (3), when IPTG is added to induce I⁺_{HQN} to unbind from the operator, the recombinase (variable) can bind and recombine the att sites, inverting the promoter resulting in the expression of GFP. To demonstrate that repressor binding to the inverted circuit does not impede GFP expression once the inducer is absent, cells in state (3) were diluted 1:200 in fresh minimal media and grown for 20 additional hours, then assayed again at state (4). General antialignment configurations are given in FIGS. 2A-2B. Data in FIGS. 20A-20D represent the average of n=6 biological replicates. Error bars correspond to the SEM of these measurements.

FIG. 21 depicts interception performance with different central dinucleotides. At left, assay data is shown for different TFs intercepting two A118 deletion circuits, one with CA central dinucleotides and an O^{ttg} operator at the P+1 position (data shown in pink) and one with AA central dinucleotides and an O^{agg} operator at the P+1 position (data shown in gray). I⁺_{HQN}, E⁺_{HQN}, and R^{+HQN} were tested for interception of the CA circuit at O^{ttg} both with and without inducer in the presence of constitutive A118 expression. I⁺_{KSL}, E⁺_{KSL}, and R⁺_{KSL} were tested for interception of the AA circuit at O^{agg} both with and without inducer in the presence of constitutive A118 expression. (inset) Modular TF components used in this study. Data represents the average of n=6 biological replicates. Error bars correspond to the SEM of these measurements.

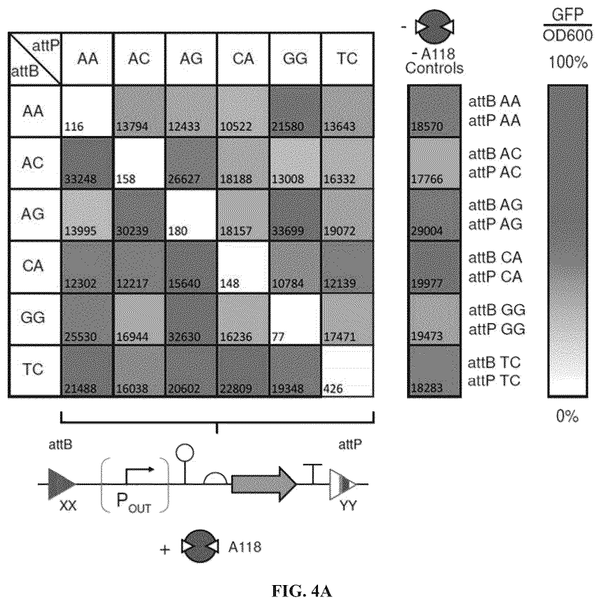

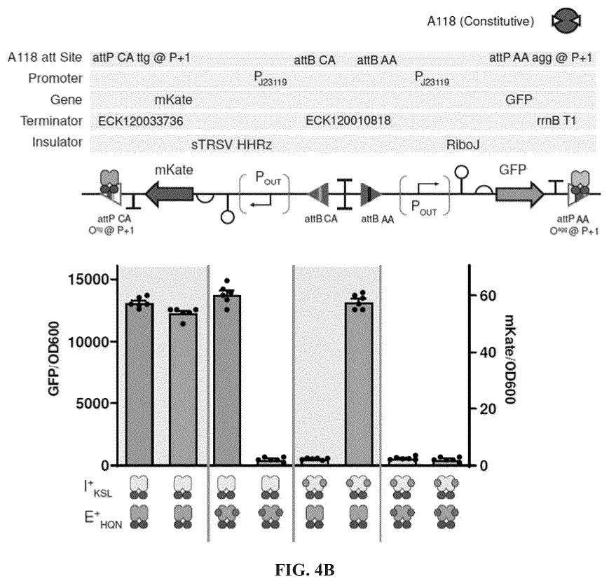

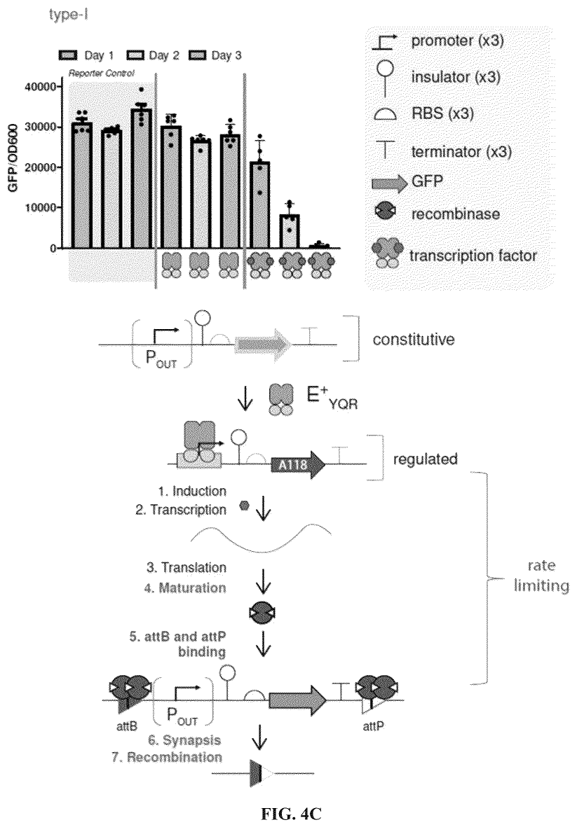

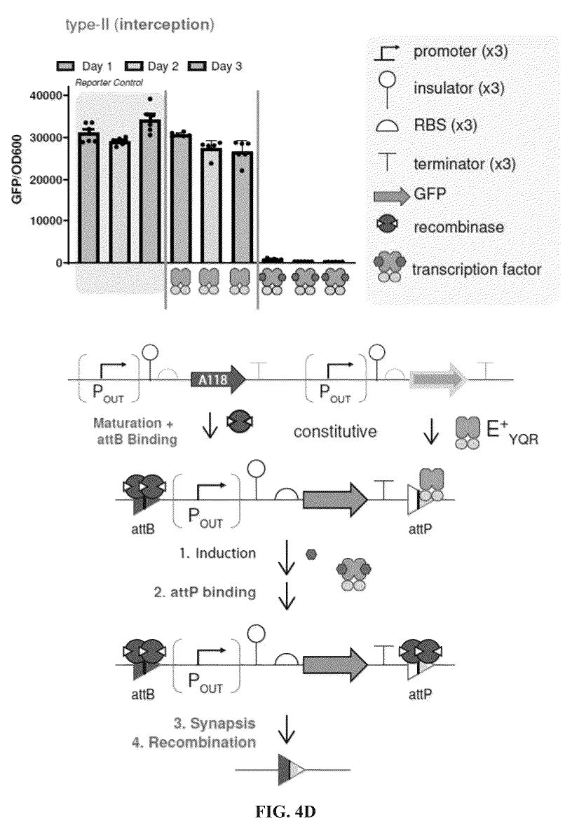

FIGS. 22A-22F depict a double-layer deletion circuit with two orthogonal attachment sites. Recombinase activities are measured with varying the central conserved region of the attachment sites. FIG. 22A shows interception was tested on reporter circuits shown in FIG. 4A for central conserved regions AA, CA, GA, AC, and TT with O^{ttg} operator inserted at the P+1 position. Each pair of bars on the plot show results for a different repressor (I⁺_{HQN}, E⁺_{HQN}, and R⁺_{HQN}) intercepting the circuit at the O^{ttg} operator in position P+1. On the left is the circuit with no ligand added (TFs are intercepting the recombinase from recombination), and on the right is the circuit with ligand added (TFs are detached from the operator, allowing recombination). A118 recombinase is constitutively expressed in all cases. FIG. 22B shows a schematic of a double-layer deletion circuit with two orthogonal attachment sites containing orthogonal O^{ttg} and O^{agg} DNA operators, enabling selective recombination: each attachment site pair with matching central conserved regions can recombine only when the ligand corresponding to their intercepting repressor is present. Repressor symbols are shown to demonstrate that these modular parts are combined in different ways to generate the data shown in FIGS. 22C-22F. FIGS. 22C-22F shows assay data for different sets of repressors targeted at the circuit shown in FIG. 22B: FIG. 22C shows E⁺_{HQN} and I⁺_{KSL}, FIG. 22D shows E⁺_{KSL} and R⁺_{HQN}, FIG. 22E shows E⁺_{HQN} and R⁺_{KSL}, and FIG. 22F shows E⁺_{KSL} and I⁺_{HQN}. Data in FIG. 22A and FIGS. 22C-22F represent the average of n=6 biological replicates. Error bars correspond to the SEM of these measurements.

FIGS. 23A-23B depict flow cytometry of a two-output circuit. FIG. 23A is a diagram showing the genetic states corresponding to the four quadrants of each plot shown in FIG. 23B. At top right, the circuit is fully protected by the intercepting TFs E⁺_{HQN} and I⁺_{KSL}. E⁺_{HQN} binds at O^{ttg} in attP site with CA central dinucleotide, protecting the red channel (mKate) deletion, and I⁺_{KSL} binds at O^{agg} in attP site with AA central dinucleotide, protecting the green channel (sfGFP) deletion. At top left, I⁺_{KSL} has been induced and sfGFP has been deleted. At bottom right, E⁺_{HQN} has been induced and mKate has been deleted. At bottom left, both TFs have been induced and both fluorescent proteins have been deleted. FIG. 23B shows flow cytometry data for each of the four inducer states, at top left no inducer, at top right+IPTG, at bottom left+cellobiose, and at bottom right+both IPTG and cellobiose. See FIGS. 11A-11H for the individual performances of I⁺_{KSL} and E⁺_{HQN} when intercepting a single-output deletion circuit as quantified by flow cytometry.

FIGS. 24A-24G depict a comparison of type-I memory and type-II memory kinetics. FIG. 24A inset at top shows a schematic of the type-I memory circuit in which A118 transcription is regulated by TFs (I⁺_{YQR}, R⁺_{YQR}, or E⁺_{YQR}) binding at O^{gtg} operators placed at the distal and proximal promoter positions. Inducing the repressing TF enables recombinase transcription and reporter circuit deletion. For kinetic assays, each TF is transformed with the A118-expression plasmid and reporter plasmid having O^{agg} at the P+1 position (O^{agg} is orthogonal to O^{gtg}, so the TFs are not expected to intercept at that operator). Below, kinetic assay data over three days is shown for this type-I memory circuit being repressed by (at left) I⁺_{YQR}, (at center) R⁺_{YQR}, and (at right) E⁺_{YQR}. On each plot, control data for cells containing only the GFP reporter plasmid is shown at left boxed in gray. The center three bars on each plot represent the circuit with no inducer added and correspond to A118 transcription being repressed over three days. The three bars at right represent the circuit with inducer added and correspond to A118 transcription being on for three days. FIG. 24B inset at top shows a schematic of the type-II memory circuit in which A118 function is intercepted by TFs (I⁺_{YQR}, R⁺_{YQR}, or E⁺_{YQR}) binding at an O^{gtg} operator placed in the P+1 position. For kinetic assays, each TF is transformed with the A118-expression plasmid and reporter plasmid. Inducing the intercepting TF enables recombinase access to the attachment sites and reporter circuit deletion. Below, kinetic assay data over three days is shown for this type-II memory circuit being intercepted by (at left) I⁺_{YQR}, (at center) R⁺_{YQR}, and (at right) E⁺_{YQR}. On each plot, control data for cells containing only the GFP reporter plasmid is shown at left boxed in gray. The center three bars on each plot represent the circuit with no inducer added and correspond to A118 function being intercepted over three days. The three bars at right represent the circuit with inducer added and correspond to A118 being unintercepted for three days. FIGS. 24C-24D display identical schematics to FIGS. 24A-24B, and the constructs are subjected to equivalent experimental conditions on day 1. Following day 1, the cells are passaged as described in PART 1 but no inducers are added, preserving the memory state written on day 1. FIGS. 24E-24G show plate reader kinetic data for type-I and type-II memory. FIG. 24E at top shows schematics for the type-I memory circuits used, addressed in different experiments by I⁺_{YQR}⁺, R⁺_{YQR}, or E⁺_{YQR}. Cells were transformed with the relevant TF and an orthogonal deletion circuit (with O^{agg} at P+1) and assayed for fluorescence every 10 minutes. FIG. 24F at top shows schematics for the type-II memory circuits used, addressed in different experiments by I⁺_{YQR}, R⁺_{YQR}, or E⁺_{YQR}. Cells were transformed with the relevant TF and a constitutive pSK001 A118-expression and assayed for fluorescence every 10 minutes. Data represents the average of n=6 biological replicates. Error bars correspond to the SEM of these measurements.

FIG. 25 depicts interception via TetR. At top, a schematic is shown for TetR intercepting a GFP deletion circuit, and the TetO operator added to A118's P+1 position is shown. At bottom, assay data for the above circuit. The leftmost bar is a control with reporter plasmid and no A118 or TetR expression. The other bars display data for all three functional plasmids being present and varying concentrations of TetR's inducer, aTc. Source data are provided as a Source Data file. Data represents the average of n=6 biological replicates. Error bars correspond to the SEM of these measurements. Sequences in FIG. 25:

(SEQ ID NO: 101)
TTAGTTCCTCGTTTTCTCTCGTTAAtccctatcagtgatagagaCTAAAC
tccctatcagtgatagaga.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The term “biocompatible” generally refers to a material and any metabolites or degradation products thereof that are generally non-toxic to the recipient and do not cause significant adverse effects to the subject.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

A “control” is an alternative subject or sample used in an experiment for comparison purposes. A control can be “positive” or “negative.”

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Encoding” refers to the inherent property of specific sequences of nucleotides in a polynucleotide, such as a gene, a cDNA, or an mRNA, to serve as templates for the synthesis of other polymers and macromolecules in biological processes having either a defined sequence of nucleotides (i.e., rRNA, tRNA, and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional properties, such as to remove or add amino acids capable of disulfide bonding, to increase its bio-longevity, to alter its secretory characteristics, etc. In any case, the fragment must possess a bioactive property, such as regulating the transcription of the target gene.

As used herein, the term “functional fragment” refers to any partial segment of a protein or nucleic acid sequence which at least partially retains the capability to perform a function or a part of a function of the full protein or full nucleic acid sequence. The functional fragment can be capable of performing multiple functions of the full protein or full nucleic acid sequence, a single function of the full protein or full nucleic acid sequence, or a part of one or more functions of the full protein or full nucleic acid sequence.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence, as referred to herein, may be used interchangeably with the term “gene”, or may include any coding sequence, a non-coding sequence or control sequence, fragments thereof, and combinations thereof. The term “gene” or “gene sequence” includes, for example, control sequences upstream of the coding sequence (for example, the ribosome binding site).

The term “increased” or “increase” as used herein generally means an increase by a statically significant amount; for example, “increased” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced,” “reduce,” “reduction,” or “decrease,” as used herein, generally means a decrease by a statistically significant amount. However, for the avoidance of doubt, “reduced” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared, can be determined by known methods.

For sequence comparisons, typically, one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high-scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

The term “polypeptide” refers to a compound made up of a single chain of D- or L-amino acids or a mixture of D- and L-amino acids joined by peptide bonds.

The term “polynucleotide” refers to a single or double-stranded polymer composed of nucleotide monomers.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

A “transcription factor” refers to a sequence-specific DNA-binding protein that controls the rate of transcription of genetic information from DNA to messenger RNA, by binding to a specific DNA sequence.

As used herein, a “transcription terminator” or a “terminator” refers to a segment of a nucleic acid sequence that marks the end of gene in genomic DNA during the transcription process, or gene expression. This sequence mediates or signals the end of transcription by providing signaling nucleotides in newly synthesized RNA transcripts that trigger an RNA polymerase to release the DNA and newly synthesized RNA.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer, or during prevention or mitigation of cancer relapse. Prophylactic administration can occur for several minutes to months prior to the manifestation of an infection.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules that comprise a Rec3 clamp, as described below.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain-relative (either internally, upstream (5′), downstream (3′), or both internally 5′ and 3′, or any combination thereof) to those domains typical of a Cas endonuclease.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (an RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracer mate sequence that hybridizes to a tracrRNA), fused to a tracrRNA (trans-activating CRISPR RNA).

### Synthetic Memory System and Methods of Use

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

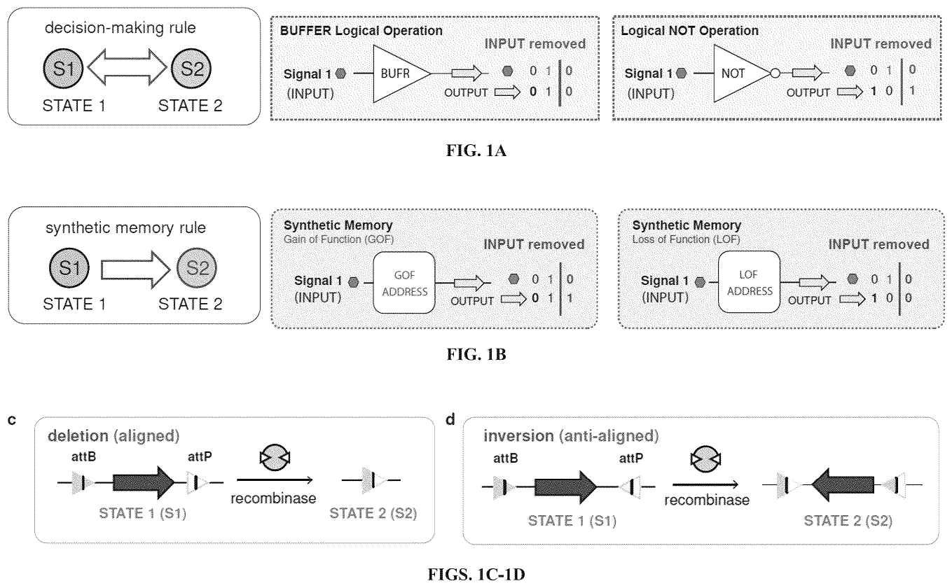

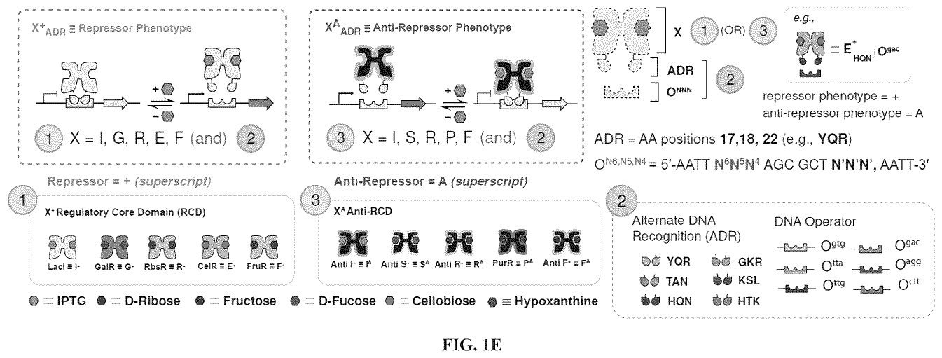

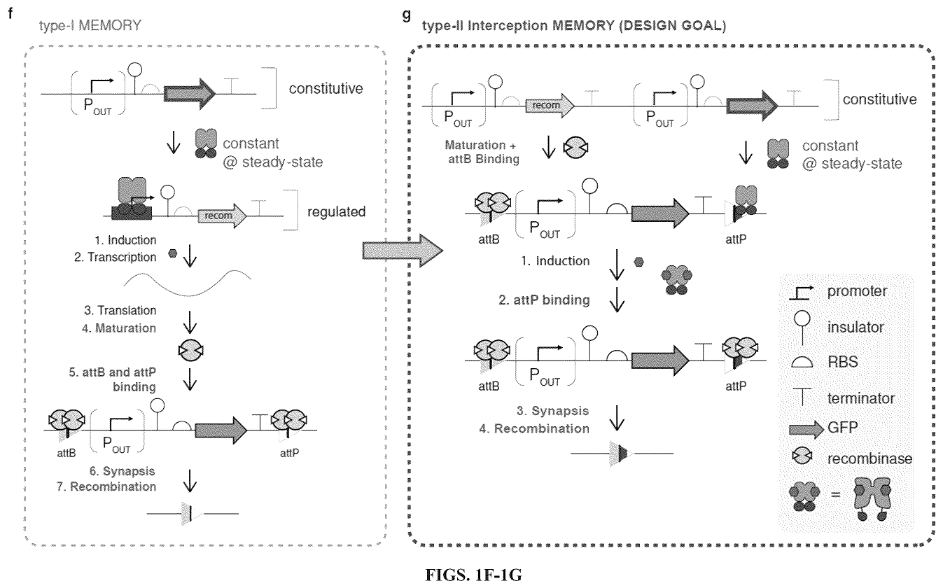

Serine integrase-based memory can be strategically paired with Transcriptional Programming (i.e., a version of decision-making) to facilitate the development of intelligent chassis cells. An intelligent biotic system is defined as one or more chassis cells capable of (i) decision-making, (ii) coupled memory development, (iii) and—for advanced systems—communication between chassis cells and/or the host. Briefly, Transcriptional Programming leverages a system of synthetic transcription factors and cognate DNA operators that can be paired with promoter elements to form inducible systems, FIG. 1E. The synthetic transcription factors are unique in that two phenotypes can be engineered (repressors denoted as superscript+ and anti-repressors denoted as superscript A), and all transcription factors can be networked via shared DNA binding functions. Each synthetic transcription factor can be modularly designed from two fundamental parts: (i) a regulatory core domain (RCD), and (ii) an engineered DNA binding domain—i.e., alternate DNA recognition (ADR) motif, FIG. 1E. Each RCD can be abbreviated using a single letter via the nomenclature defined in Swint-Kruse et al. [24], e.g., LacI=I, GalR=G, RbsR=R, CelR=E, FruR=F. Each ADR is cognate to an engineered operator DNA element. The engineered DNA binding functions are predicated on ADR position 17, 18 and 22 (e.g., residues Y17, Q18, and R22 are abbreviated as YQR) being concurrently varied and paired with one or more putative symmetric operator DNA variant(s) with substitutions at positions 6, 5, and 4 in the left half-site of the DNA operator. A putative DNA operator variant is defined as 5′-AATT N⁶N⁵N⁴ AGCGCT N′N′N′ AATT-3′ (SEQ ID NO: 1) where N^{#} is any nucleotide and N′ represents the nucleotide required to make the operator fully symmetric²⁵. Therefore, the engineered operator can be abbreviated as O^{N6,N5,N4}, see FIG. 1E. Accordingly, DNA binding domain TAN pairs with operator DNA element O^{tta}—whereas HQN pairs with O^{ttg} and so forth. To simplify the interpretation of the pairing of an engineered ADR with a DNA operator all cognate sets have been color-coded, see FIG. 1E.

Canonical synthetic memory (type-I) is achieved by way of the regulation of a given recombinase, which is typically induced by a small molecule. Once matured (folded and assembled) the recombinase attaches to DNA elements attB and attP and results in reconfiguration of DNA, see FIG. 1F. Disclosed herein is a post-translational strategy for controlling recombinase function, termed interception (type-II), that expands the utility of recombinases for synthetic memory operations. Interception is defined as the controlled blocking of any protein-DNA interaction (other than RNA polymerase) via a transcription factor (TF) that interacts with a cognate DNA operator pair in situ. In recombinase systems, interception can be achieved via strategically replacing a small segment of a recombinase attachment site with a DNA operator—see FIG. 1G. Mechanistically this would result in the TF—when bound to operator DNA—sterically hindering a given recombinase from binding to a cognate attachment site. Correspondingly, under conditions in which the TF becomes unbound (i.e., induced) the said recombinase can attach to the DNA element and catalyze the reconfiguration of cognate DNA elements (e.g., resulting in deletion or inversion—illustration and iconography given in FIGS. 1C-1D and FIGS. 2A-2B). Accordingly, this iteration of synthetic memory requires two parts: (i) an operation that regulates recombinase attachment post-translation, and (ii) a genetic address to define the memory function—i.e., the orientation and positioning of recombinase attachment sites attB and attP.

In an aspect, provided is a synthetic memory system, including: a recombinase; a modified transcription factor; and a first nucleic acid including a first attachment site, a second attachment site, and a target gene between said first attachment site and said second attachment site; wherein at least one of the first attachment site and the second attachment site includes a modified DNA operator to which the modified transcription factor can reversibly bind; and wherein, when the modified transcription factor is bound to the modified DNA operator, the recombinase is blocked from binding to the attachment site including said modified DNA operator.

A “recombinase,” as used herein, is a site-specific enzyme that mediates the recombination of DNA between recombinase recognition sequences, which results in the excision, integration, inversion, or exchange (e.g., translocation) of DNA fragments between the recombinase recognition sequences (e.g., attachment sites). Recombinases can be classified into two distinct families: serine recombinases and tyrosine recombinases. In some aspects, the recombinase can be a serine integrase. For example, in some such aspects, the recombinase can be A118, TP901, Bxb1, Int2, Int3, Int5, Int8, or Int12. In other aspects, the recombinase can be a tyrosine integrase. For example, in some such aspects, the recombinase can be Cre, FLP, R, Lambda, HK101, HK022, or pSAM2. In some aspects, a functional fragment of a recombinase can be used.

In some aspects, the modified transcription factor can include at least one DNA binding domain and at least one regulatory core domain. For example, in some aspects, the modified transcription factor can include any of the regulatory core domains, DNA binding domains, and/or combinations thereof as described in U.S. Patent Application Publication No. 2024/0229021 A9, which is hereby incorporated by reference in its entirety. In some aspects, the at least one DNA binding domain can include about 80% identity or more (e.g., about 81% identity or more, about 82% identity or more, about 83% identity or more, about 84% identity or more, about 85% identity or more, about 86% identity or more, about 87% identity or more, about 88% identity or more, about 89% identity or more, about 90% identity or more, about 91% identity or more, about 92% identity or more, about 93% identity or more, about 94% identity or more, about 95% identity or more, about 96% identity or more, about 97% identity or more, about 98% identity or more, about 99% identity or more) to any one of SEQ ID NO: 71, 73, 75, 77, 79, 81, or 91. In some aspects, the at least one DNA binding domain can include any one of SEQ ID NO: 71, 73, 75, 77, 79, 81, or 91.

TABLE 6
Example DNA binding domains.

| Sequence | SEQ ID NO |
|---|---|
| AEYAGVSYQTVSRVVNQASHV | 71 |
| AEYAGVSGKTVSRVVNQASHV | 73 |
| AEYAGVSTATVSNVVNQASHV | 75 |
| AEYAGVSHQTVSNVVNQASHV | 77 |
| AEYAGVSHTTVSKVVNQASHV | 79 |
| AEYAGVSKSTVSLVVNQASHV | 81 |
| AEYAGVSFKTVSRVVNQASHV | 91 |

In some aspects, the modified transcription factor can be encoded by a nucleic acid including about 80% identity or more (e.g., about 81% identity or more, about 82% identity or more, about 83% identity or more, about 84% identity or more, about 85% identity or more, about 86% identity or more, about 87% identity or more, about 88% identity or more, about 89% identity or more, about 90% identity or more, about 91% identity or more, about 92% identity or more, about 93% identity or more, about 94% identity or more, about 95% identity or more, about 96% identity or more, about 97% identity or more, about 98% identity or more, about 99% identity or more) to any one of SEQ ID NOs: 102-111. In some aspects, the modified transcription factor can be encoded by a nucleic acid including any one of SEQ ID NOs. 102-111. In some such aspects, the DNA binding domain of the transcription factor can be modified by altering residues 49-54 and 64-66 of any one of SEQ ID NOs: 102-111 (shown in bold in TABLE 7).

TABLE 7
Example modified transcription factors.

| Name | Sequence | SEQ ID NO |
|---|---|---|
| LacI | GTGAAACCAGTAACGTTATACGATGTCGCAGAGTATGCCGGTGTC | 102 |
| | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | GAATTACATTCCCAACCGCGTGGCACAACAACTGGCGGGCAAACA | |
| | GTCGTTGCTGATTGGCGTTGCCACCTCCAGTCTGGCCCTGCACGCG | |
| | CCGTCGCAAATTGTCGCGGCGATTAAATCTCGCGCCGATCAACTGG | |
| | GTGCCAGCGTGGTGGTGTCGATGGTAGAACGAAGCGGCGTCGAAG | |
| | CCTGTAAAACGGCGGTGCACAATCTTCTCGCGCAACGCGTCAGTG | |
| | GGCTGATCATTAACTATCCGCTGGATGACCAGGATGCCATTGCTGT | |
| | GGAAGCTGCCTGCACTAATGTTCCGGCGTTATTTCTTGATGTCTCT | |
| | GACCAGACACCCATCAACAGTATTATTTTCTCCCATGAAGACGGTA | |
| | CGCGACTGGGCGTGGAGCATCTGGTCGCATTGGGTCACCAGCAAA | |
| | TCGCGCTGTTAGCGGGCCCATTAAGTTCTGTCTCGGCGCGTCTGCG | |
| | TCTGGCTGGCTGGCATAAATATCTCACTCGCAATCAAATTCAGCCG | |
| | ATAGCGGAACGGGAAGGCGACTGGAGTGCCATGTCCGGTTTTCAA | |
| | CAAACCATGCAAATGCTGAATGAGGGCATCGTTCCCACTGCGATG | |
| | CTGGTTGCCAACGATCAGATGGCGCTGGGCGCAATGCGCGCCATT | |
| | ACCGAGTCCGGGCTGCGCGTTGGTGCGGATATCTCGGTAGTGGGA | |
| | TACGACGATACCGAAGACAGCTCATGTTATATCCCGCCGTTAACCA | |
| | CCATCAAACAGGATTTTCGCCTGCTGGGGCAAACCAGCGTGGACC | |
| | GCTTGCTGCAACTCTCTCAGGGCCAGGCGGTGAAGGGCAATCAGC | |
| | TGTTGCCCGTCTCACTGGTGAAAAGAAAAACCACCCTGGCGCCCA | |
| | ATACGCAAACCGCCTCTCCCCGCGCGTTGGCCGATTCATTAATGCA | |
| | GCTGGCACGACAGGTTTCCCGACTGGAAAGCGGGCAGTGA | |
| CelR | GTGAAACCAGTAACGTTATACGATGTCGCAGAGTATGCCGGTGTC | 103 |
| | TCTTATCAGACCGTTTCCCGCGTGGTGAACCAGGCCAGCCACGTT | |
| | TCTGCGAAAACGCGGGAAAAAGTGGAAGCGGCGATCAAAGAGCT | |
| | GGGCTACGTGCCGAACCGCGCAGCCCGCACCCTGGTCACCCGACG | |
| | TACCGACACCGTAGCCCTGGTGGTGTCGGAAAACAACCAGAAGCT | |
| | CTTCGCCGAACCCTTCTATGCCGGGATCGTGCTCGGCGTGGGGGTT | |
| | GCTCTGTCCGAACGGGGATTCCAGTTCGTCCTGGCCACGGGCCGCT | |
| | CCGGGATAGAGCATGAGCGGCTGGGCGGCTACCTGGCCGGACAGC | |
| | ACGTCGACGGGGTCCTCCTGCTGTCGCTCCACCGCGACGACCCGCT | |
| | GCCGCAGATGCTGGACGAGGCCGGGGTGCCGTACGTCTACGGCGG | |
| | CCGTCCGCTCGGCGTCCCCGAAGAACAGGTGTCCTATGTCGATATC | |
| | GACAACATCGGCGGGGGACGCCAGGCCACCCAGCGGCTGATCGAG | |
| | ACCGGGCACCGGCGGATCGCTACGATCGCGGGCCCGCAGGACATG | |
| | GTCGCTGGTGTGGAACGCCTCCAGGGGTATCGCGAAGCACTGCTC | |
| | GCCGCGGGGATGGAGTACGACGAGACGCTGGTGAGCTACGGTGAC | |
| | TTCACCTACGACAGCGGGGTGGCCGCGATGCGGGAGCTGCTGGAT | |
| | CGGGCCCCCGACGTGGACGCCGTGTTCGCGGCCTCCGACTTGATG | |
| | GGGCTGGCCGCGCTGCGGGTGCTGCGTGCTTCGGGACGCCGCGTG | |
| | CCCGAGGATGTGGCGGTGGTCGGCTACGACGACTCGACCGTAGCC | |
| | GAGCACGCCGAACCGCCGATGACCAGCGTCAACCAGCCCACCGAG | |
| | CTGATGGGCCGGGAGATGGCCCGGCTGCTCGTGGACCGGATCACC | |
| | GGGGAGACCACCGAACCGGTGCGGCTGGTGCTGGAGACCCATTTG | |
| | ATGGTGCGGGAATCCGGGTGA | |
| RbsR | GTGAAACCAGTAACGTTATACGATGTCGCAGAGTATGCCGGTGTC | 104 |
| | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CAATTACATTCCCAACCGCGTGGCACAACAACTGGCGGGCAAAGC | |
| | GtcgCATacaATTGGCATGTTGATCACTGCCAGTACCAATCCTTTCTAT | |
| | TCAGAACTGGTGCGTGGCGTTGAACGCAGCTGCTTCGAACGCGGT | |
| | TATAGTCTCGTCCTTTGCAATACCGAAGGCGATGAACAGCGGATG | |
| | AATCGCAATCTGGAAACGCTGATGCAAAAACGCGTTGATGGCTTG | |
| | CTGTTACTGTGCACCGAAACGCATCAACCTTCGCGTGAAATCATGC | |
| | AACGTTATCCGACAGTGCCTACTGTGATGATGGACTGGGCTCCGTT | |
| | CGATGGCGACAGCGATCTTATTCAGGATAACTCGTTGCTGGGCGG | |
| | AGACTTAGCAACGCAATATCTGATCGATAAAGGTCATACCCGTAT | |
| | CGCCTGTATTACCGGCCCGCTGGATAAAACTCCGGCGCGCCTGCG | |
| | GTTGGAAGGTTATCGGGCGGCGATGAAACGTGCGGGTCTCAACAT | |
| | TCCTGATGGCTATGAAGTCACTGGTGATTTTGAATTTAACGGCGGG | |
| | TTTGACGCTATGCGCCAACTGCTATCACATCCGCTGCGTCCTCAGG | |
| | CCGTCTTTACCGGAAATGACGCTATGGCTGTTGGCGTTTACCAGGC | |
| | GTTATATCAGGCAGAGTTACAGGTTCCGCAGGATATCGCGGTGATT | |
| | GGCTATGACGATATCGAACTGGCAAGCTTTATGACGCCACCATTA | |
| | ACCACTATCCACCAACCGAAAGATGAACTGGGGGAGCTGGCGATT | |
| | GATGTACTCATCCATCGGATAACCCAGCCGACCCTTCAGCAACAA | |
| | CGATTACAACTTACTCCGATTCTGATGGAACGCGGTTCGGCTTAG | |
| GalR | GTGAAACCAGTAACGTTATACGATGTCGCAGAGTATGCCGGTGTC | 105 |
| | TCTTATCAGACCGTTTCCCGCGTGGTGAACCAGGCCAGCCACGTT | |
| | TCTGCGAAAACGCGGGAAAAAGTGGAAGCGGCGATGGAGTCTCTT | |
| | AGCTATCACCCGAACGCCAACGCCCGTGCGCTGGCGCAGCAGACC | |
| | ACTGAAACGGTCGGTCTGGTCGTTGGTGATGTTTCCGATCCGTTTT | |
| | TCGGTGCAATGGTGAAAGCGGTCGAACAGGTGGCTTATCACACCG | |
| | GTAATTTTTTATTGATTGGCAACGGTTACCACAACGAACAAAAAG | |
| | AGCGTCAGGCCATTGAGCAACTGATCCGCCATCGCTGTGCTGCGTT | |
| | GGTCGTCCATGCCAAAATGATCCCGGATGCTGATTTAGCCTCATTA | |
| | ATGAAACAAATGCCCGGTATGGTGCTGATCAACCGTATCCTGCCTG | |
| | GCTTTGAAAACCGTTGTATTGCTCTGGACGATCGTTACGGTGCCTG | |
| | GCTGGCAACGCGTCATTTAATTCAGCAAGGTCATACCCGCATTGGT | |
| | TATCTGTGCTCTAACCACTCTATTTCTGACGCCGAAGATCGTCTGC | |
| | AAGGGTATTACGATGCCCTTGCTGAAAGTGGTATTGCGGCCAATG | |
| | ACCGGCTGGTGACATTTGGCGAACCAGACGAAAGCGGCGGCGAAC | |
| | AGGCAATGACCGAGCTTTTGGGACGAGGAAGAAATTTCACTGCGG | |
| | TAGCCTGTTATAACGATTCAATGGCGGCGGGTGCGATGGGCGTTCT | |
| | CAATGATAATGGTATTGATGTACCGGGTGAGATTTCGTTAATTGGC | |
| | TTTGATGATGTGCTGGTGTCACGCTATGTGCGTCCGCGCCTGACCA | |
| | CCGTGCGTTACCCAATCGTGACGATGGCGACCCAGGCTGCCGAAC | |
| | TGGCTTTGGCGCTGGCGGATAATCGCCCTCTCCCGGAAATCACTAA | |
| | TGTCTTTAGTCCGACGCTGGTACGTCGTCATTCAGTGTCAACTCCG | |
| | TCGCTGGAGGCAAGTCATCATGCAACCAGCGACTAA | |
| FruR | GTGAAACCAGTAACGTTATACGATGTCGCAGAGTATGCCGGTGTC | 106 |
| | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CAATTACATTCCCAACCGCGTGGCACAACAACTGGCGGGCAAAAG | |
| | TAGCCGTTCTATTGGTCTTGTGATCCCCGATCTGGAGAACACCAGC | |
| | TATACCCGCATCGCTAACTATCTTGAACGCCAGGCGCGGCAACGG | |
| | GGTTATCAACTGCTGATTGCCTGCTCAGAAGATCAGCCAGACAAC | |
| | GAAATGCGGTGCATTGAGCACCTTTTACAGCGTCAGGTTGATGCCA | |
| | TTATTGTTTCGACGTCGTTGCCTCCTGAGCATCCTTTTTATCAACGC | |
| | TGGGCTAACGACCCGTTCCCGATTGTCGCGCTGGACCGCGCCCTCG | |
| | ATCGTGAACACTTCACCAGCGTGGTTGGTGCCGATCAGGATGATG | |
| | CCGAAATGCTGGCGGAAGAGTTACGTAAGTTTCCCGCCGAGACGG | |
| | TGCTTTATCTTGGTGCGCTACCGGAGCTTTCTGTCAGCTTCCTGCGT | |
| | GAACAAGGTTTCCGTACTGCCTGGAAAGATGATCCGCGCGAAGTG | |
| | CATTTCCTGTATGCCAACAGCTATGAGCGGGAGGCGGCTGCCCAG | |
| | TTATTCGAAAAATGGCTGGAAACGCATCCGATGCCGCAGGCGCTG | |
| | TTCACAACGTCGTTTGCGTTGTTGCAAGGAGTGATGGATGTCACGC | |
| | TGCGTCGCGACGGCAAACTGCCTTCTGACCTGGCAATTGCCACCTT | |
| | TGGCGATAACGAACTGCTCGACTTCTTACAGTGTCCGGTGCTGGCA | |
| | GTGGCTCAACGTCACCGCGATGTCGCAGAGCGTGTGCTGGAGATT | |
| | GTCCTGGCAAGCCTGGACGAACCGCGTAAGCCAAAACCTGGTTTA | |
| | ACGCGCATTAAACGTAATCTCTATCGCCGCGGCGTGCTCAGCCGTA | |
| | GCTAA | |
| Anti- | GTGAAACCAGTAACGTTATACGATGTCGCAGAGTATGCCGGTGTC | 102 |
| LacI | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | GAATTACATTCCCAACCGCGTGGCACAACAACTGGCGGGCAAACA | |
| | GTCGTTGCTGATTGGCGTTGCCACCTCCAGTCTGGCCCTGCACGCG | |
| | CCGTCGCAAATTGTCGCGGCGATTAAATCTCGCGCCGATCAACTGG | |
| | GTGCCAGCGTGGTGGTGTCGATGGTAGAACGAAGCGGCGTCGAAG | |
| | CCTGTAAAACGGCGGTGCACAATCTTCTCGCGCAACGCGTCAGTG | |
| | GGCTGATCATTAACTATCCGCTGGATGACCAGGATGCCATTGCTGT | |
| | GGAAGCTGCCTGCACTAATGTTCCGGCGTTATTTCTTGATGTCTCT | |
| | GACCAGACACCCATCAACAGTATTATTTTCTCCCATGAAGACGGTA | |
| | CGCGACTGGGCGTGGAGCATCTGGTCGCATTGGGTCACCAGCAAA | |
| | TCGCGCTGTTAGCGGGCCCATTAAGTTCTGTCTCGGCGCGTCTGCG | |
| | TCTGGCTGGCTGGCATAAATATCTCACTCGCAATCAAATTCAGCCG | |
| | ATAGCGGAACGGGAAGGCGACTGGAGTGCCATGTCCGGTTTTCAA | |
| | CAAACCATGCAAATGCTGAATGAGGGCATCGTTCCCACTGCGATG | |
| | CTGGTTGCCAACGATCAGATGGCGCTGGGCGCAATGCGCGCCATT | |
| | ACCGAGTCCGGGCTGCGCGTTGGTGCGGATATCTCGGTAGTGGGA | |
| | TACGACGATACCGAAGACAGCTCATGTTATATCCCGCCGTTAACCA | |
| | CCATCAAACAGGATTTTCGCCTGCTGGGGCAAACCAGCGTGGACC | |
| | GCTTGCTGCAACTCTCTCAGGGCCAGGCGGTGAAGGGCAATCAGC | |
| | TGTTGCCCGTCTCACTGGTGAAAAGAAAAACCACCCTGGCGCCCA | |
| | ATACGCAAACCGCCTCTCCCCGCGCGTTGGCCGATTCATTAATGCA | |
| | GCTGGCACGACAGGTTTCCCGACTGGAAAGCGGGCAGTGA | |
| Anti- | GTGAAACCAGTAACGTTATACGATGTCGCAGAGTATGCCGGTGTC | 107 |
| RbsRA | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CAATTACATTCCCAACCGCGTGGCACAACAACTGGCGGGCAAAGC | |
| | GTCGCATACCATTGGCATGTTGATCACTGCCAGTACCAATCCTTTC | |
| | TATTCAGAACTGGTGCGTGGCGTTGAACGCAGCTGCTTCGAACGC | |
| | GGTTATAGTCTCGATCTTTGCAATACCGAAGGCGATGAACAGCGG | |
| | ATGAATCGCAATCTGGAAACGCTGATGCAAAAACGCGTTGATGGC | |
| | TTGCTGTTACTGTGCACCGAAACGCATCAACCTTCGCGTGAAATCA | |
| | TGCAACGTTATCCGACAGTGCCTACTGTGATGATGGACTGGGCTCC | |
| | GTTCGATGGCGACAGCGATCTTATTCAGGATAACTCGTTGCTGGGC | |
| | GGAGACTTAGCAACGCAATATCTGATCGATAAAGGTCATACCCGT | |
| | ATCGCCTGTATTACCGGCCCGCTGGATAAAACTCCGGCGCGCCTGC | |
| | GGTTGGAAGGTTATCGGGCGGCGATGAAACGTGCGGGTCTCAACA | |
| | TTCCTGATGGCTATGAAGTCACTGGTGATTTTGAATTTAACGGCGG | |
| | GTTTGACGCTATGCGCCAACTGCTATCACATCCGCTGCGTCCTCAG | |
| | GCCGTCTTTACCGGAAATGACGCTATGGCTGTTGGCGTTTACCAGG | |
| | CGTTATATCAGGCAGAGTTACAGGTTCCGCAGGATATCGCGGTGA | |
| | TTGGCTATGACGATATCGAACTGGCAAGCTTTATGACGCCACCATT | |
| | AACCACTATCCACCAACCGAAAGATGAACTGGGGGAGCTGGCGAT | |
| | TGATGTACTCATCCATCGGATAACCCAGCCGACCCTTCAGCAACAA | |
| | CGATTACAACTTACTCCGATTCTGATGGAACGCGGTTCGGCTTAG | |
| Anti- | GTGAAACCAGTAACGTTATACGATGTCGCAGAGTATGCCGGTGTC | 108 |
| FruR | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CAATTACATTCCCAACCGCGTGGCACAACAACTGGCGGGCAAAAG | |
| | TTCGCGTTCTATTGGCCTTGTGATCCCCGATCTGGAGAACACCAGC | |
| | TATTCCCGCTTCGCTAACTATCTCGAACGCCAGGCGCGGCAACGGG | |
| | GTTATCAACTGAAAATTGCCTGCTCAGAAGATCAGCCAGACAACG | |
| | AAATGCGGTGCATTGAGCACCTTTTACAGCGTCAGGTTGATGCCAT | |
| | TATTGTTTCGACGTCGTTGCCTCCTGAGCATCCTTTTTATCAACGCT | |
| | GGGCTAACGACCCGTTCCCGATTGTCGCGCTGGACCGCGCCCTCGA | |
| | TCGTGAACACTTCACCAGCGTGGTTGGTGCCGATCAGGATGATGCC | |
| | GAAATGCTGGCGGAAGAGTTACGTAAGTTTCCCGCCGAGACGGTG | |
| | CTTTATCTTGGTGCGCTACCGGAGCTTTCTGTCAGCTTCCTGCGTGA | |
| | ACAAGGTTTCCGTACTGCCTGGAAAGATGATCCGCGCGAAGTGCA | |
| | TTTCCTGTATGCCAACAGCTATGAGCGGGAGGCGGCTGCCCAGTTA | |
| | TTCGAAAAATGGCTGGAAACGCATCCGATGCCGCAGGCGCTGTTC | |
| | ACAACGTCGTTTGCGTTGTTGCAAGGAGTGATGGATGTCACGCTGC | |
| | GTCGCGACGGCAAACTGCCTTCTGACCTGGCAATTGCCACCTTTGG | |
| | CCAGAACGAACTGCTCGACTTCTTACAGTGTCCGGTGCTGGCAGTG | |
| | GCTCAACGTCACCGCGATGTCGCAGAGCGTGTGCTGGAGATTGTC | |
| | CTGGCAAGCCTGGACGAACCGCGTAAGCCAAAACCTGGTTTAACG | |
| | CGCATTAAACGTAATCTCTATCGCCGCGGCGTGCTCAGCCGTAGCT | |
| | AA | |
| Anti- | GTGAAACCAGTAACGTTATACGATGTCGCAGAGTATGCCGGTGTC | 109 |
| GalS | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CAATTACATTCCCAACCGCGTGGCAGCCCAACTGGCGGGCAAACA | |
| | GTCGGACACCATAGGCGTGGTGGTGATGGACGTTACTGATGCGTTT | |
| | TTCGGCGCGCTGGTAAAAGCGGTGGATCAGGTCGCTCAGCAGCAT | |
| | CAGAAATACGTGGCAATCGGCAATAGCTATCATGAAGCGGAAAAA | |
| | GAGCGTCACGCCATTGAGGTGTTAATTCGCCAGCGTTGTAATGCGT | |
| | TGATTGTTCACTCAAAAGCATTGAGTGACGATGAACTGGCGCAATT | |
| | TATGGATAACATTCCCGGTATGGTGTTAATCAACCGCGTTGTGCCG | |
| | GGGTACGCCCATCGTTGCGTTTGTCTGGATAATCTCAGCGGTGCCC | |
| | GAATGGCGACGCGCATGTTGCTGAATAACGGTCATCAACGTATTG | |
| | GTTATCTTTCTTCCAGCCACGGCATTGAAGATGACGCCATGCGTAA | |
| | AGCAGGCTGGATGAGTGCGTTGAAAGAGCAGGATATTATTCCGCC | |
| | GGAAAGCTGGATTGGCGCTGGTACGCCGGACATGCCGGGCGGTGA | |
| | GGCGGCGTTGGTTAAACTGCTGGGGCGCAATCTACAACTTACCGCT | |
| | GTATTTGCTTATAACGACAATATGGCCGCTGGCGCACTGACAGCAT | |
| | TAAAAGATAATGGCATTGCGATTCCGTTACATCTCTCAATCATCGG | |
| | GTTCGATGATATTCCCATCGCCCGTTACACCGACCCGCAATTAACG | |
| | ACCGTGCGTTATCCCATTGCTTCAATGGCGAAATTAGCCACCGAAC | |
| | TGGCCTTGCAGGGGGCAGCAGGCAATATTGATCCTCGTGCCAGCC | |
| | ACTGTTTTATGCCGACGTTAGTGCGTCGCCATTCTGTCGCAACACG | |
| | CCAGAATGCGGCGGCGATCACTAACTCAACAAATCAGGCGATGTA | |
| | A | |
| PurR | GTGAAACCAGTAACGTTATACGATGTCGCAGAGTATGCCGGTGTC | 110 |
| | TCTTATCAGACCGTTTCCCGCGTGGTGAACCAGGCCAGCCACGTT | |
| | TCTGCGAAAACGCGGGAAAAAGTGGAAGCGGCGATGGCGGAGCT | |
| | CCACTACATTCCTAGCGCGGTGGCGCGTAGCCTGGCGGGCAAACA | |
| | GTCGAAGTCTATCGGTTTGCTGGCGACCAGCAGCGAAGCGGCCTA | |
| | TTTTGCCGAGATCATTGAAGCAGTTGAAAAAAATTGCTTCCAGAA | |
| | AGGTTACACCCTGATTCTGGGCAATGCGTGGAACAATCTTGAGAA | |
| | ACAGCGGGCTTATCTGTCGATGATGGCGCAAAAACGCGTCGATGG | |
| | TCTGCTGGTGATGTGTTCTGAGTACCCAGAGCCGTTGCTGGCGATG | |
| | CTGGAAGAGTATCGCCATATCCCAATGGTGGTCATGGACTGGGGT | |
| | GAAGCAAAAGCTGACTTCACCGATGCGGTCATTGATAACGCGTTC | |
| | GAAGGCGGCTACATGGCCGGGCGTTATCTGATTGAACGCGGTCAC | |
| | CGCGAAATCGGCGTCATCCCCGGCCCGCTGGAACGTAACACCGGC | |
| | GCAGGCCGCCTTGCCGGTTTTATGAAGGCGATGGAAGAAGCGATG | |
| | ATCAAGGTGCCGGAAAGCTGGATTGTGCAGGGTGACTTTGAACCT | |
| | GAATCCGGTTATCGCGCCATGCAGCAAATCCTGTCGCAGCCGCATC | |
| | GCCCTACTGCCGTCTTCTGTGGTGGCGATATCATGGCAATGGGCGC | |
| | ACTTTGTGCTGCTGATGAAATGGGCCTGCGCGTCCCGCAGGATGTT | |
| | TCGCTGATCGGTTATGATAACGTGCGCAACGCGCGCTATTTTACGC | |
| | CGGCGCTGACCACGATCCATCAGCCAAAAGATTCGCTGGGTGAAA | |
| | CAGCGTTCAACATGCTGTTGGATCGTATCGTCAACAAACGTGAAG | |
| | AACCGCAGTCTATTGAAGTGCATCCGCGCTTGATTGAACGCCGCTC | |
| | CGTGGCTGACGGCCCGTTCCGCGACTATCGTCGTTAA | |
| TetR | ATGTCCAGATTAGATAAAAGTAAAGTGATTAACAGCGCATTAGAG | 111 |
| | CTGCTTAATGAGGTCGGAATCGAAGGTTTAACAACCCGTAAACTC | |
| | GCCCAGAAGCTAGGTGTAGAGCAGCCTACATTGTATTGGCATGTA | |
| | AAAAATAAGCGGGCTTTGCTCGACGCCTTAGCCATTGAGATGTTA | |
| | GATAGGCACCATACTCACTTTTGCCCTTTAGAAGGGGAAAGCTGG | |
| | CAAGATTTTTTACGTAATAACGCTAAAAGTTTTAGATGTGCTTTAC | |
| | TAAGTCATCGCGATGGAGCAAAAGTACATTTAGGTACACGGCCTA | |
| | CAGAAAAACAGTATGAAACTCTCGAAAATCAATTAGCCTTTTTATG | |
| | CCAACAAGGTTTTTCACTAGAGAATGCATTATATGCACTCAGCGCT | |
| | GTGGGGCATTTTACTTTAGGTTGCGTATTGGAAGATCAAGAGCATC | |
| | AAGTCGCTAAAGAAGAAAGGGAAACACCTACTACTGATAGTATGC | |
| | CGCCATTATTACGACAAGCTATCGAATTATTTGATCACCAAGGTGC | |
| | AGAGCCAGCCTTCTTATTCGGCCTTGAATTGATCATATGCGGATTA | |
| | GAAAAACAACTTAAATGTGAAAGTGGGTCCTAA | |

In some aspects, the modified transcription factor can respond to addition or removal of an input by modifying binding of said transcription factor. For example, in some aspects, adding the input can cause the modified transcription factor to reversibly bind to the modified DNA operator and removing the input can cause the modified transcription factor to release from the modified DNA operator (e.g., anti-repressor phenotype). In other aspects, adding the input can cause the modified transcription factor to release from the modified DNA operator and removing the input can cause the modified transcription factor to reversibly bind to the modified DNA operator (e.g., repressor phenotype).

In some aspects, the input can be a small molecule. For example, in some such aspects, the input can be a sugar, such as isopropyl-β-D-thiogalactopyrano side, D-ribose, fructose, D-fucose, cellobiose, hypoxanthine, or any combination thereof.

In some aspects, each of the first attachment site and the second attachment site can include a conserved region and two half-sites on either side of said conserved region.

In some aspects, the conserved region can be at least 2 nucleotides (e.g., at least 3 nucleotides, at least 4 nucleotides, at least 5 nucleotides, at least 6 nucleotides, at least 7 nucleotides, at least 8 nucleotides, at least 9 nucleotides, at least 10 nucleotides) in length. In some aspects, the conserved region can be up to 10 nucleotides (e.g., up to 9 nucleotides, up to 8 nucleotides, up to 7 nucleotides, up to 6 nucleotides, up to 5 nucleotides, up to 4 nucleotides, up to 3 nucleotides, up to 2 nucleotides) in length.

It is considered that the conserved region can range in length from any of the minimum values described above to any of the maximum values described above. For example, in some aspects, the conserved region can be from 2 nucleotides to 10 nucleotides (e.g., from 3 nucleotides to 9 nucleotides, from 4 nucleotides to 8 nucleotides, from 5 nucleotides to 7 nucleotides, from 2 nucleotides to 6 nucleotides, from 3 nucleotides to 5 nucleotides, from 6 nucleotides to 10 nucleotides, from 7 nucleotides to 9 nucleotides) in length.

In some aspects, each half-site can independently be at least 20 nucleotides (e.g., at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, at least 36 nucleotides, at least 37 nucleotides, at least 38 nucleotides, at least 39 nucleotides, at least 40 nucleotides) in length. In some aspects, each half-site can independently be up to 40 nucleotides (e.g., up to 39 nucleotides, up to 38 nucleotides, up to 37 nucleotides, up to 36 nucleotides, up to 35 nucleotides, up to 34 nucleotides, up to 33 nucleotides, up to 32 nucleotides, up to 31 nucleotides, up to 30 nucleotides, up to 29 nucleotides, up to 28 nucleotides, up to 27 nucleotides, up to 26 nucleotides, up to 25 nucleotides, up to 24 nucleotides, up to 23 nucleotides, up to 22 nucleotides, up to 21 nucleotides, up to 20 nucleotides) in length.

It is considered that each half-site can independently range in length from any of the minimum values described above to any of the maximum values described above. For example, in some aspects, each half-site can independently be from 20 nucleotides to 40 nucleotides (e.g., from 21 nucleotides to 39 nucleotides, from 22 nucleotides to 38 nucleotides, from 23 nucleotides to 37 nucleotides, from 24 nucleotides to 36 nucleotides, from 25 nucleotides to 35 nucleotides, from 26 nucleotides to 34 nucleotides, from 27 nucleotides to 33 nucleotides, from 28 nucleotides to 32 nucleotides, from 29 nucleotides to 31 nucleotides, from 20 nucleotides to 30 nucleotides, from 21 nucleotides to 29 nucleotides, from 22 nucleotides to 28 nucleotides, from 23 nucleotides to 27 nucleotides, from 24 nucleotides to 26 nucleotides, from 30 nucleotides to 40 nucleotides, from 31 nucleotides to 39 nucleotides, from 32 nucleotides to 38 nucleotides, from 33 nucleotides to 37 nucleotides, from 34 nucleotides to 36 nucleotides) in length.

In some aspects, each half-site (e.g., before addition of the modified DNA operator) can be the same. In other aspects, each half-site (e.g., before addition of the modified DNA operator) can be different. For example, in some such aspects, each half-site can be about 80% palindromic or more (e.g., about 81% palindromic or more about 82% palindromic or more, about 83% palindromic or more, about 84% palindromic or more, about 85% palindromic or more, about 86% palindromic or more, about 87% palindromic or more, about 88% palindromic or more, about 89% palindromic or more, about 90% palindromic or more, about 91% palindromic or more, about 92% palindromic or more, about 93% palindromic or more, about 94% palindromic or more, about 95% palindromic or more, about 96% palindromic or more, about 97% palindromic or more, about 98% palindromic or more, about 99% palindromic or more, 100% palindromic).

In some aspects, the modified DNA operator can be upstream of the conserved region. In other aspects, the modified DNA operator can be downstream of the conserved region. In some aspects, the modified DNA operator can be spaced from the conserved region by up to 10 nucleotides (e.g., up to 9 nucleotides, up to 8 nucleotides, up to 7 nucleotides, up to 6 nucleotides, up to 5 nucleotides, up to 4 nucleotides, up to 3 nucleotides, up to 2 nucleotides, up to 1 nucleotide, 0 nucleotides). In some aspects, the modified DNA operator can be inserted between residues of the half-site. In other aspects, the modified DNA operator can overwrite residues of the half-site corresponding to some or all of the length of the modified DNA operator.

In some aspects, the modified DNA operator can be at least 10 nucleotides (e.g., at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides) in length. In some aspects, the modified DNA operator can be up to 20 nucleotides (e.g., up to 19 nucleotides, up to 18 nucleotides, up to 17 nucleotides, up to 16 nucleotides, up to 15 nucleotides, up to 14 nucleotides, up to 13 nucleotides, up to 12 nucleotides, up to 11 nucleotides, up to 10 nucleotides) in length.

It is considered that the modified DNA operator can range in length from any of the minimum values described above to any of the maximum values described above. For example, in some aspects, the modified DNA operator can be from 10 nucleotides to 20 nucleotides (e.g., from 11 nucleotides to 19 nucleotides, from 12 nucleotides to 18 nucleotides, from 13 nucleotides to 17 nucleotides, from 14 nucleotides to 16 nucleotides, from 10 nucleotides to 15 nucleotides, from 11 nucleotides to 14 nucleotides, from 12 nucleotides to 13 nucleotides, from 15 nucleotides to 20 nucleotides, from 16 nucleotides to 19 nucleotides, from 17 nucleotides to 18 nucleotides) in length.

In some aspects, the modified DNA operator can include about 80% identity or more (e.g., about 81% identity or more, about 82% identity or more, about 83% identity or more, about 84% identity or more, about 85% identity or more, about 86% identity or more, about 87% identity or more, about 88% identity or more, about 89% identity or more, about 90% identity or more, about 91% identity or more, about 92% identity or more, about 93% identity or more, about 94% identity or more, about 95% identity or more, about 96% identity or more, about 97% identity or more, about 98% identity or more, about 99% identity or more) to any one of SEQ ID NOs: 53, 70, 72, 74, 76, 78, or 80. In some aspects, the modified DNA operator can include any one of SEQ ID NOs: 53, 70, 72, 74, 76, 78, or 80.

TABLE 8
Example modified DNA operators.

| Sequence | SEQ ID NO |
|---|---|
| TTTTGAGCGCTCAAAA | 53 |
| ATTGTGAGCGCTCACAATT | 70 |
| ATTGACAGCGCTGTCAATT | 72 |
| ATTTTAAGCGCTTAAAATT | 74 |
| ATTTTGAGCGCTCAAAATT | 76 |
| ATTCTTAGCGCTAAGAATT | 78 |
| ATTAGGAGCGCTCCTAATT | 80 |

In some aspects, the first attachment site can be an attB site, and the second attachment site can be an attP site. In other aspects, the first attachment site can be an attP site, and the second attachment site can be an attB site. In some aspects, the first attachment site can include the modified DNA operator. In other aspects, the second attachment site can include the modified DNA operator. In yet other aspects, the first attachment site and the second attachment site can each include modified DNA operators (e.g., same or different), and two modified transcription factors (e.g., same or different) can be used. It is understood that, as described herein for consistency, the first attachment site is upstream of the second attachment site.

In some aspects, recombination can occur when a first recombinase binds to the first attachment site and a second recombinase binds to the second attachment site. In some such aspects, the first recombinase and the second recombinase can be the same or different. In some aspects, the first attachment site and the second attachment site can be aligned such that, upon recombination, the target gene is deleted. In other aspects, the first attachment site and the second attachment site can be anti-aligned such that, upon recombination, the target gene is inverted.

In some aspects, each of the first attachment site and the second attachment site can be selected based on the recombinase used. For example, in some such aspects, the wildtype (e.g., before addition of the modified DNA operator) attB sites and wildtype attP sites corresponding to several recombinases are given in TABLE 9.

TABLE 9
Example wildtype attB and attP sites for given recombinases.

| Recombinase | attB Site | attP Site |
|---|---|---|
| A118 | AACTTTTCGGATCAAGCTATGAA | TTAGTTCCTCGTTTTCTCTCGTTAA |
| | AAACGCAAAGAGGGAACTAAAC | AAGAAGAAGAAACGAGAAACTAA |
| | ACTT (SEQ ID NO: 5) | A (SEQ ID NO: 6) |
| TP901 | ATGCCAACACAATTAACATCTCA | AGCGAGTTTTTATTTCGTTTATTTC |
| | ATCAAGGTAAATGCTTTTTGCTTT | AATTAAGGTAACTAAAAAACTCCT |
| | TTTTGC (SEQ ID NO: 13) | TT (SEQ ID NO: 16) |
| Int3 | GTTTGTAAAGGAGACTGATAATG | ATGGATAAAAAAATACAGCGTTTT |
| | GCATGTACAACTATACTCGTCGG | TCATGTACAACTATACTAGTTGTAG |
| | TAAAAAGGCATCTTAT (SEQ ID | TGCCTAAATAATGCTT (SEQ ID NO: |
| | NO: 19) | 22) |
| Int5 | GAGCGCCGGATCAGGGAGTGGAC | CCCTAATACGCAAGTCGATAACTCT |
| | GGCCTGGGAGCGCTACACGCTGT | CCTGGGAGCGTTGACAACTTGCGC |
| | GGCTGCGGTCGGTGC (SEQ ID NO: | ACCCTGATCTG (SEQ ID NO: 28) |
| | 25) | |
| Int8 | CAATCATCAGATAACTATGGCGG | TTAATAAACTATGGAAGTATGTAC |
| | CACGTGCATTAACCACGGTTGTA | AGTCTTGCAATGTTGAGTGAACAA |
| | TCCCGTCTAAAGTACTCGT (SEQ | ACTTCCATAATAAAAT (SEQ ID NO: |
| | ID NO: 31) | 34) |
| Int12 | GTTCGTGGTAACTATGGGTGGTA | TTTTTGTATGTTAGTTGTGTCACTG |
| | CAGGTGCCACATTAGTTGTACCA | GGTAGACCTAAATAGTGACACAAC |
| | TTTATGTTTATGTGGTTAAC (SEQ | TGCTATTAAAATTTAA (SEQ ID NO: |
| | ID NO: 36) | 39) |
| Bxb1 | CGGCCGGCTTGTCGACGACGGCG | GTCGTGGTTTGTCTGGTCAACCACC |
| | GTCTCCGTCGTCAGGATCATCCG | GCGGTCTCAGTGGTGTACGGTACA |
| | GGC (SEQ ID NO: 42) | AACCCCGAC (SEQ ID NO: 44) |
| Int2 | GGACGGCGCAGAAGGGGAGTAG | GCTCATGTATGTGTCTACGCGAGAT |
| | CTCTTCGCCGGACCGTCGACATA | TCTCGCCCGAGAACTTCTGCAAGG |
| | CTGCTCAGCTCGTC (SEQ ID NO: | CACTGCTCTTGGCT (SEQ ID NO: 50) |
| | 47) | |

In some aspects, the first attachment site can include about 80% identity or more (e.g., about 81% identity or more, about 82% identity or more, about 83% identity or more, about 84% identity or more, about 85% identity or more, about 86% identity or more, about 87% identity or more, about 88% identity or more, about 89% identity or more, about 90% identity or more, about 91% identity or more, about 92% identity or more, about 93% identity or more, about 94% identity or more, about 95% identity or more, about 96% identity or more, about 97% identity or more, about 98% identity or more, about 99% identity or more) to any one of SEQ ID NOs: 54-60, 65-69, 82-90, or 92-100. In some aspects, the first attachment site can include any one of SEQ ID NOs: 54-60, 65-69, 82-90, or 92-100.

In some aspects, the second attachment site can include about 80% identity or more (e.g., about 81% identity or more, about 82% identity or more, about 83% identity or more, about 84% identity or more, about 85% identity or more, about 86% identity or more, about 87% identity or more, about 88% identity or more, about 89% identity or more, about 90% identity or more, about 91% identity or more, about 92% identity or more, about 93% identity or more, about 94% identity or more, about 95% identity or more, about 96% identity or more, about 97% identity or more, about 98% identity or more, about 99% identity or more) to any one of SEQ ID NOs: 54-60, 65-69, 82-90, or 92-100. In some aspects, the second attachment site can include any one of SEQ ID NOs: 54-60, 65-69, 82-90, or 92-100.

TABLE 10
Example attachment sites.

| Sequence | SEQ ID NO |
|---|---|
| TTAGTTCCTCGTTTTCTCTCGTTAATTTTGAGCGCTCAAAAAAACTAAA | 54 |
| TTTTGAGCGCTCAAAATCTCGTTAAAAGAAGAAGAAACGAGAAACTAAA | 55 |
| TTAGTTTTTTGAGCGCTCAAAATAAAAGAAGAAGAAACGAGAAACTAAA | 56 |
| TTAGTTCCTTTTTGAGCGCTCAAAAAAGAAGAAGAAACGAGAAACTAAA | 57 |
| TTAGTTCCTCTTTTGAGCGCTCAAAAAGAAGAAGAAACGAGAAACTAAA | 58 |
| TTAGTTCCTCGTTTTCTCTCGTTAAAATTTTGAGCGCTCAAAAACTAAA | 59 |
| TTAGTTCCTCGTTTTCTCTCGTTAAAAGTTTTGAGCGCTCAAAACTAAA | 60 |
| TTAGTTCCTCGTTTTCTCTCGTTAATTCTTAGCGCTAAGAAAAACTAAA | 65 |
| TTAGTTCCTCGTTTTCTCTCGTTAATTGACAGCGCTGTCAAAAACTAAA | 66 |
| TTAGTTCCTCGTTTTCTCTCGTTAATTAGGAGCGCTCCTAAAAACTAAA | 67 |
| TTAGTTCCTCGTTTTCTCTCGTTAATTTTAAGCGCTTAAAAAAACTAAA | 68 |
| TTAGTTCCTCGTTTTCTCTCGTTAATTGTGAGCGCTCACAAAAACTAAA | 69 |
| GCTCATGTATGTGTCTACGCGAGATTCTCGCCTTGTGAGCGCTCACAAGCAC | 82 |
| TGCTCTTGGCT | |
| CCCTAATACGCAAGTCGATAACTCTCCTGGGAGCGTTTTGAGCGCTCAAAAC | 83 |
| CTGATCTG | |
| TTTTTGTATGTTAGTTGTGTCACTGGGTAGACTTGTGAGCGCTCACAACTGC | 84 |
| TATTAAAATTTAA | |
| GTCGTGGTTTGTCTGGTCAACCACCGCGGTTTTTGAGCGCTCAAAAACAAAC | 85 |
| CCCGAC | |
| AGCGAGTTTTTATTTCGTTTATTTCTTTTGAGCGCTCAAAAAAACTCCTTT | 86 |
| GTTTGTAAAGGAGACTGATAATGGCATGTACAACTATACTTTGTGAGCGCTC | 87 |
| ACAATCTTAT | |
| ATGGATAAAAAAATACAGCGTTTTTCATGTACAACTATACTTTGTGAGCGCT | 88 |
| CACAATAATGCTT | |
| TTAATAAACTATGGAAGTATGTACAGTCTTGCAATTGTGAGCGCTCACAATT | 89 |
| CCATAATAAAAT | |
| TTAATAAACTATGGAATTGTGAGCGCTCACAAAATGTTGAGTGAACAAACT | 90 |
| TCCATAATAAAAT | |
| AGTTTTGAGCGCTCAAAATTATTTCAATTAAGGTAACTAAAAAACTCCTTT | 92 |
| AGCGAGTTTTTGAGCGCTCAAAATCAATTAAGGTAACTAAAAAACTCCTTT | 93 |
| AGCGAGTTTTTATTTCGTTTATTTCAATTTTGAGCGCTCAAAAACTCCTTT | 94 |
| ATGGATAAAAAAATACAGCGTTTTTCATGTACAACTATACTATTTTGAGCGC | 95 |
| TCAAAAAATGCTT | |
| ATGGATAAAAAAATACAGCGTTTTTCATGTACAACTATACTAGTTTTGAGCG | 96 |
| CTCAAAAATGCTT | |
| ATGGATAAAAAAATACAGCGTTTTTCATGTACAACTATACTAGTTGTTTTGA | 97 |
| GCGCTCAAAACTT | |
| ATGGATAAAAAAATACAGCGTTTTTCATGTACAACTATACTAGTTGTTTTTG | 98 |
| AGCGCTCAAAATT | |
| CCCTATTTTGAGCGCTCAAAACTCTCCTGGGAGCGTTGACAACTTGCGCACC | 99 |
| CTGATCTG | |
| TTTTTGTATGTTAGTTGTGTCACTGGGTAGACCTAAATAGTGTTTTGAGCGCT | 100 |
| CAAAAAATTTAA | |

In some aspects, a first modified transcription factor and a second modified transcription factor can simultaneously reversibly bind to the modified DNA operator, and, when at least one of the first modified transcription factor and the second modified transcription factor is bound to the modified DNA operator, the recombinase can be blocked from binding to the attachment site including said modified DNA operator. In some aspects, the first modified transcription factor and the second modified transcription factor can be the same and/or respond to the same input. In other aspects, the first modified transcription factor and the second modified transcription factor can be different and/or respond to different inputs. In some aspects, both the first modified transcription factor and the second modified transcription factor can have the same phenotype (e.g., repressor or anti-repressor). In other aspects, the first modified transcription factor and the second modified transcription factor can have different phenotypes. It is understood that, in some aspects, more than two modified transcription factors can simultaneously reversibly bind to the modified DNA operator, for example, as described in U.S. Patent Application Publication No. 2024/0229021 A9.

In some aspects, the synthetic memory system can further include a second nucleic acid encoding the recombinase and a third nucleic acid encoding the modified transcription factor. In some aspects, any combination of the first nucleic acid, the second nucleic acid, and the third nucleic acid can be linked together.

In another aspect, provided is a method of executing a memory operation, the method including: a) providing a synthetic memory system including a recombinase; a modified transcription factor; and a first nucleic acid including a first attachment site, a second attachment site, and a target gene between said first attachment site and said second attachment site; wherein at least one of the first attachment site and the second attachment site includes a modified DNA operator to which the modified transcription factor can reversibly bind; and wherein, when the modified transcription factor is bound to the modified DNA operator, the recombinase is blocked from binding to the attachment site including said modified DNA operator; and b) adding or removing an input; wherein the modified transcription factor responds to the addition or removal of said input by modifying binding of said transcription factor and triggering the memory operation.

In some aspects, the synthetic memory system can be any of the disclosed synthetic memory systems.

In some aspects, adding the input can cause the modified transcription factor to reversibly bind to the modified DNA operator and removing the input can cause the modified transcription factor to release from the modified DNA operator (e.g., anti-repressor phenotype). In other aspects, adding the input can cause the modified transcription factor to release from the modified DNA operator and removing the input can cause the modified transcription factor to reversibly bind to the modified DNA operator (e.g., repressor phenotype).

In some aspects, the memory operation can be a loss-of-function (e.g., deletion) of the target gene. In other aspects, the memory operation can be a gain-of-function (e.g., inversion) of the target gene.

### Examples

### Example 1: Methods

Strains and media: Standard DNA cloning was performed via chemical transformation in NEB DH5-α Chemically Competent Escherichia coli (huA2 Δ(argF⁻lacZ)U169 phoA glnV44φ80Δ(lacZ) M15 gyrA96 recA1 relA1 endA1 thi-1 hsdR17; New England Biolabs (NEB)), and assay experiments were performed via chemical transformation in E. coli strain 3.32 (lacZ13(Oc) lacI22λ⁻ el4-relA1 spoT thiE1; Yale CGSC #5237). For transformations, cells were grown in SOC medium (Fisher Scientific); media for growing anti-repressor constructs required supplementation with ligands as described below. For precultures prior to assays, cells were recovered in Luria Broth (LB) Miller Medium (Fisher Scientific) supplemented with appropriate antibiotics; again, media for growing antirepressor constructs was supplemented with appropriate ligands. Assays were performed in 1× M9 Minimal Medium (6.8 g L⁻¹ Na₂HPO₄, 3.0 g L⁻¹ KH₂PO₄, 0.5 g L⁻¹ NaCl, 1.0 g L⁻¹ NH₄Cl, 2 mM MgSO₄, 100 M CaCl₂; Millipore Sigma) supplemented with 0.2% (w/v) casamino acids (VWR Life Sciences), 1 mM thiamine HCl (Alfa Aesar), and 0.4% (w/v) glucose. LB Miller Agar (Fisher Scientific) was used for selection during cloning. Antibiotics and ligands were used where appropriate. Antibiotics used were: chloramphenicol (25 g mL⁻¹; VWR Life Sciences), kanamycin (35 g mL⁻¹; VWR Life Sciences), and carbenicillin (100 g mL⁻¹; Teknova). Ligands used were: adenine (as a precursor to the ligand for PurR, hypoxanthine [36], Acros Organics), cellobiose (Arcos Organics), D-fucose (Carbosynth), D-ribose (Arcos Organics), D-fructose (Arcos Organics), and isopropyl-β-D-thiogalactoside (IPTG; Millipore Sigma). Adenine (hypoxanthine) was supplemented in assay and antirepressor growth media (including SOC for transformations and LB Miller agar selection plates) at 1 mM concentration; all other ligands were added to assay media at 10 mM concentration.

Cloning and plasmid construction: For all cloning experiments, oligomer and genestrand synthesis and DNA sequencing were performed by Eurofins Genomics. All DNA plasmids were purified via miniprep (Omega Bio-Tek) and sequenced to verify correct assembly. A Plasmid Editor (ApE) and SnapGene software were used to facilitate primer design and sequence alignments. Polymerase chain reactions (PCR) were performed using Phusion High-Fidelity PCR Master Mix with HF Buffer or GC Buffer (NEB), or using Q5 Polymerase Master Mix (NEB) on a C1000 Touch Thermal Cycler (Bio-Rad). BsaI-HF™v2 restriction enzyme (NEB) was used to construct operator position libraries, and BfuAI restriction enzyme (NEB) was used to construct three-TF plasmids as described below. AvrII restriction enzyme (NEB), AatII restriction enzyme (NEB), NdeI restriction enzyme (NEB), and PacI restriction enzyme (NEB) were used to tune the expression of E⁺ and I⁺ on the two-TF plasmids used for nested transcriptional logic circuits as described below.

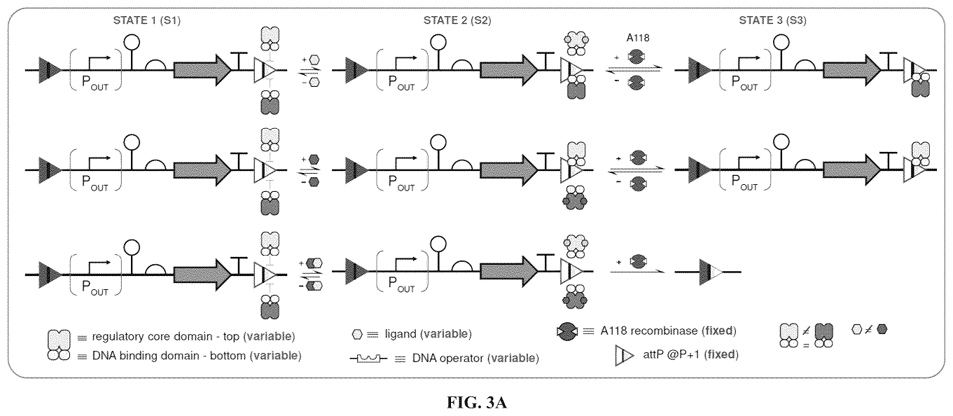

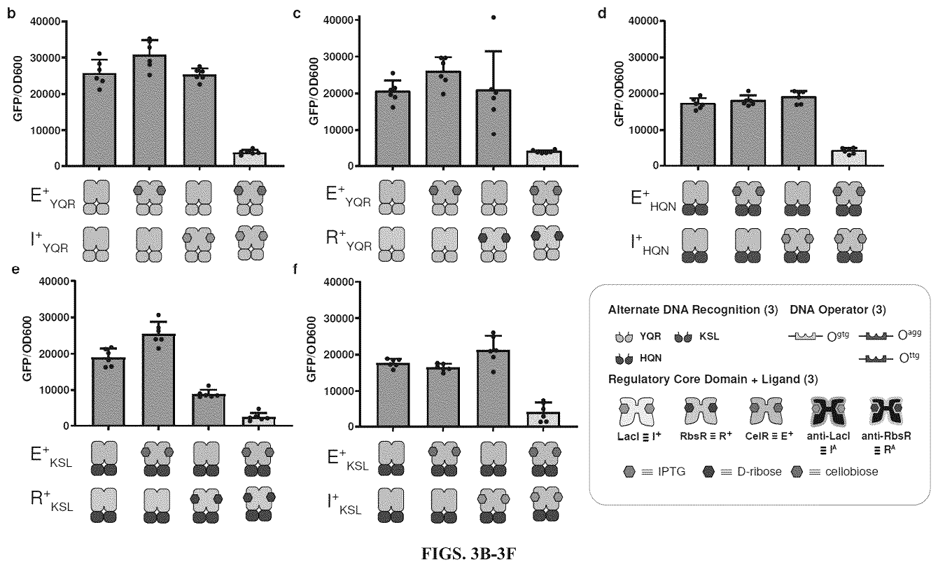

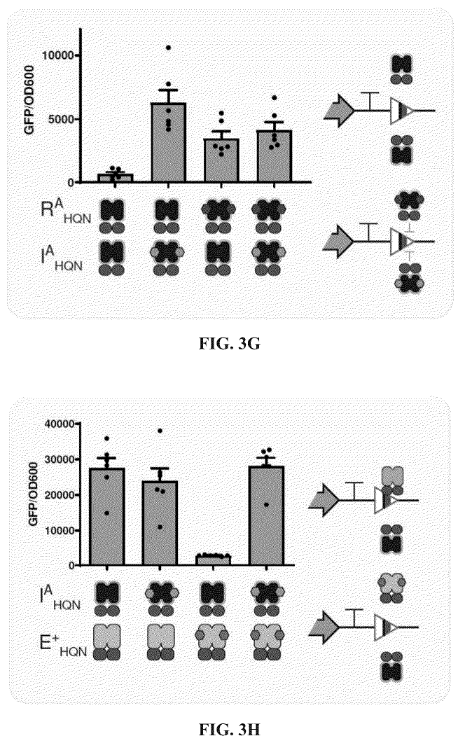

Reporter plasmid constructs: The reporter plasmids containing the deletion circuit architecture (shown in FIG. 1A) were constructed starting with the pZS*22-sfGFP plasmid reported in Richards et. al., featuring a low-copy-number pSC101* origin of replication and kanamycin resistance. The J23119 promoter and RiboJ segments were ordered as gene fragments from Eurofins Genomics and used to replace the LacIQ promoter on pZS*22 via Gibson assembly (NEB HIFI enzymes). Similarly, the two attachment sites (attB and attP) complementary to each recombinase tested were ordered as gene fragments from Eurofins Genomics and added upstream and downstream of sfGFP via Gibson assembly (NEB HIFI enzymes). As described in FIGS. 1A-1G, minimal 16 bp LacI-family operators were added in the place of attachment site DNA via site-directed mutagenesis using Phusion DNA polymerase with HF buffer (NEB), facilitated by the NEBuilder software followed by treatment with KLD Enzyme Mix (NEB). Attachment half-site deletions were performed via PCR followed by treatment with KLD enzyme mix. For half-sites “B2” and “P1” (see FIGS. 1A-1G), random DNA spacers were added in place of the B2 and P1 attachment half-sites; these were designed using UCR's Random Sequence Generator Tool online. To construct the “nested-logic” reporter construct (shown in FIGS. 3A-3H), the reporter plasmid containing the deletion circuit architecture for the A118 recombinase described in “Recombinase-expression constructs” was linearized to delete the J23119 promoter. Alternative promoter regions based on the pTrc promoter but modified to include the O_{sym} YQR operator in either the “core” or “proximal” position were amplified from plasmids available in the lab, developed for Rondon et al. [3]. These alternative promoters were added to the reporter plasmid via Gibson assembly (NEB HIFI) and sequence verified.

Recombinase-expression constructs: Recombinase-expression plasmids were constructed using a pLacI (Novagen) backbone featuring a medium-copy-number p15A origin and chloramphenicol resistance. A118, Bxb1, and TP901 recombinases were independently amplified for Gibson assembly (NEB HIFI) from the pNR220 plasmid (a gift from the Lu lab at MIT), int2, int5, and int8 were amplified for Gibson assembly from the pCis_2+7+8+5 plasmid (Addgene 60588), int3 was amplified from pIntegrase_3 (Addgene 60575), and int12 was amplified from pIntegrase_12 (Addgene 60583) [12]. The expression level of recombinases was tuned using RBS tuning and promoter tuning. RBS tuning was facilitated by the Salis lab's RBS Library Calculator [38], [39], and performed using site-directed mutagenesis with variable-codon primer tails (Eurofins Genomics). Variable-strength promoters were taken from Wang et. al., ordered as genestrands from Eurofins genomics, and added via Gibson assembly.

Transcription-factor-expression constructs: Transcription-factor-expression plasmids were constructed using a pTB146 backbone (a gift from the Xiong lab at Yale University) containing a medium-high copy number ColE1 origin and M13 origin (with copy number suppressed by the Rop protein) and ampicillin resistance. Chimeric transcription factors were sourced in-house from plasmids developed by Rondon et al. [3], [36] and Groseclose et al. [2], [35] and added downstream of a constitutive LacI promoter via Gibson assembly (NEB HIFI). F⁺ and F^{A(1)} were placed downstream of the constitutive LacIQ promoter to increase the transcription rate about 10 fold. Mutations to the DNA-binding domains were introduced as needed via site-directed mutagenesis using Phusion DNA polymerase with HF buffer (NEB) targeting nucleotides outlined in Rondon et. al. Plasmids containing two TFs were constructed via Gibson assembly (NEB HIFI). Plasmids containing three TFs were constructed by amplifying individual TFs using primers containing BfuAI recognition sites (NEB) from single-TF plasmids, subcloning these in a pUC19 cloning vector (NEB N3041S) via Gibson assembly (NEB HIFI), and assembling the three parts into one destination vector on the pTB146 backbone through Golden Gate Cloning.

Microwell plate assay: The protocol for the fluorescence-based microwell plate assay was taken from Richards et al. Each relevant plasmid was chemically transformed into 3.32 E. coli cells. Transformants were selected on LB Miller agar (Fisher Scientific) plates containing the corresponding antibiotics (and inducers at 1 mM or 10 mM, for antirepressor experiments), then six replicates were grown for 8 hours in a 96-well clear, flat-bottomed assay plate (Costar) sealed with a Breathe-Easier membrane (Midwest Scientific), shaking at 300 RPM at 37° C. in LB Miller in a Fisher shaker (Fisher Scientific MaxQ400) with appropriate antibiotics (and relevant inducers, for antirepressor experiments). 1 μL from each of the six culture replicates was then diluted in 200 μL of supplemented 1× M9 Minimal Media (containing the relevant set of antibiotics and inducers) in a 96-well clear, flat-bottomed culture plate (Costar). Plates were sealed with Breathe-Easyy membranes (Midwest Scientific) to prevent evaporation and grown for 16 hours, shaking at 300 RPM at 37° C. in a Fisher shaker (Fisher Scientific MaxQ400). 150 μL of cells from each well were transferred to a 96-well black-sided, clear-bottomed assay plate (Costar). Fluorescence and optical density (OD₆₀₀) were measured via plate reader (Molecular Devices SpectraMax M2e) using an excitation wavelength of 485 nm and an emission wavelength of 510 nm (for sfGFP reporters). Data was collected with SoftMax Pro Software (Molecular Devices). Wells containing M9 Minimal Media and relevant antibiotics and inducers with no cell inoculations were used as blanks; the average fluorescence of the six blanks for each condition was subtracted from each sample-fluorescence-intensity reading. Similarly, the average optical density of the six blanks for each condition was subtracted from each sample-optical-density reading. Blank-compensated fluorescence data was then normalized to blank-compensated optical density data for each sample, with an aim to quantify average fluorescence per cell. Data analysis was performed in Microsoft Excel and GraphPad Prism.

Two-output circuit assay: The mKate/GFP two-output circuit shown in FIG. 4B was assayed following an additional 24-hour media passage with no inducers to demonstrate maintenance of the memory state and allow the degradation of excess fluorescent proteins from cells following fluorescent protein circuit deletion. Briefly, variants were subjected to the same growth conditions given in the Microwell Plate Assay section. After the 16-hour growth step in minimal media with and without the relevant inducers, replicates were diluted 1:200 in LB Miller with appropriate antibiotics and no inducers and shaken for an additional 8 hours in a 96-well clear, flat-bottomed assay plate (Costar) sealed with a Breathe-Easier membrane (Midwest Scientific), shaking at 300 RPM at 37° C. 1 μL from each of the six culture replicates was then diluted in 200 L of supplemented 1× M9 Minimal Media (containing the relevant set of antibiotics and no inducers) in a 96-well clear, flat-bottomed culture plate (Costar). Plates were sealed with Breathe-Easy membranes (Midwest Scientific) to prevent evaporation and grown for 16 hours, shaking at 300 RPM at 37° C. in a Fisher shaker (Fisher Scientific MaxQ400). 150 μL of cells from each well were transferred to a 96-well black-sided, clear-bottomed assay plate (Costar). Fluorescence and optical density (OD₆₀₀) were measured via plate reader (Molecular Devices SpectraMax M2e) using an excitation wavelength of 485 nm and an emission wavelength of 510 nm (for sfGFP), and an excitation wavelength of 588 nm and an emission wavelength of 635 nm (for mKate).

Recombinase three-day kinetic assays: For testing the kinetics of transcriptionally-regulated A118 expression, 3.32 E. coli cells were transformed with the pSK012 transcriptionally regulated A118 plasmid along with the relevant TF-expression plasmids and the pSK153 reporter plasmid (having the O^{agg} operator, which is orthogonal to the TF's YQR DNA-binding domains, at the P+1 position). For testing the kinetics of interception-regulated A118 function, 3.32 E. coli cells were transformed with the pSK001 constitutive A118 expression plasmid along with the relevant TF-expression plasmids and the pSK148 plasmid (having the OSYM operator, which the TF's YQR DNA-binding domains bind to for interception, at the P+1 position). In both cases, data collection was performed at three timepoints labeled “Day 1”, “Day 2”, and “Day 3.” For the Day 1 timepoint, cells were precultured and assayed as described in “Microwell plate assay.” For the Day 2 and Day 3 timepoints, the same preculture and assay conditions were applied, except the cells were transferred to preculture (being diluted 1:200) in LB Miller from the previous assay plate rather than from the initial petri dishes.

Recombinase plate reader kinetic assays: For testing the kinetics of transcriptionally-regulated A118 expression, 3.32 E. coli cells were transformed with the pSK012 transcriptionally regulated A118 plasmid along with the relevant TF-expression plasmids and the pSK153 reporter plasmid (having the O^{agg} operator, which is orthogonal to the TF's YQR DNA-binding domains, at the P+1 position). For testing the kinetics of interception-regulated A118 function, 3.32 E. coli cells were transformed with the pSK001 constitutive A118 expression plasmid along with the relevant TF-expression plasmids and the pSK148 plasmid (having the OSYM operator, which the TF's YQR DNA-binding domains bind to for interception, at the P+1 position). In both cases, transformants were precultured and diluted in minimal media conditions as outlined in the “Microwell plate assay” section. Assay plates were then placed in a plate reader (Molecular Devices SpectraMax M2e) and subjected to the following kinetic assay protocol: (1) set and hold temperature at 37° C., (2) shake for 4 minutes, (3) read optical density (OD₆₀₀), (4) read fluorescence using an excitation wavelength of 485 nm and an emission wavelength of 510 nm, (5) wait 3 minutes, (6) repeat (for a total of 1,000 cycles).

Recombinase RBS library construction: Recombinase RBS libraries designed in Salis lab's RBS Library Calculator were chemically transformed in NEB DH5-α Chemically Competent Escherichia coli, and scraped from plates and collectively miniprepped, yielding one DNA solution of RBS library. Then recombinase RBS library DNA was transformed in E. coli strain 3.32 with reporter plasmids and transcription factor plasmids. To cover the variants, 96 transformants were picked and inoculated in a 96-well preculture plate (Costar) containing LB Miller with appropriate antibiotics, and shaken at 300 RPM at 37° C. in a Fisher shaker (Fisher Scientific MaxQ400) for 8 hours. A 96-pin replicator (Boekel Sci) was used to inoculate these samples in two 96-well black-sided, clear-bottomed assay plates (Costar), each containing 1× M9 Minimal Media and antibiotics, and one containing the appropriate ligand. Assay plates were sealed with Breathe-Easy membranes (Midwest Scientific) to prevent evaporation and grown for 20 hours, shaken at 300 RPM at 37° C. To characterize the performance of the recombinases, the assay plates with and without ligand were compared and analyzed. Normalized fluorescence data, as described in “Microwell plate assay,” was used to analyze performance. Recombinase plasmids were then extracted by PCR (Phusion High-Fidelity PCR Master Mix with HF Buffer (NEB)), followed by treatment with KLD Enzyme Mix (NEB). Extracted RBS variants were then sequenced and analyzed through Salis lab's RBS Library Calculator. Finally, recombinases with tuned RBS were assayed again in sixplicate to confirm the tuned recombinase performances.

Library construction for testing operator position within attP sites: Operator libraries were designed in Excel to include a unique 4 bp barcode downstream of the attachment site and ordered as oligopools from IDT. Oligopools were amplified with primers containing BsaI recognition sites to enable Golden Gate library assembly; BsaI-HF™v2 restriction enzyme (NEB) was used. The set of plasmids thus constructed was transformed into NEB 5-α Chemically Competent cells and selected on LB agar supplemented with kanamycin. Transformants were counted to quantify library coverage using equation 2 from Patrick et. al. [40]; all libraries used had expected coverage >95%. Transformants were then scraped from plates and collectively miniprepped, yielding one DNA solution containing the library. The cells were grown as described in “Recombinase RBS library construction.” EasyFluorescence measurements were recorded as described in the “Microwell plate assay” section. Samples exhibiting target phenotypes were individually grown in LB Miller and sequenced; the barcode was used to identify operator position. Positions thus identified were assayed in sixplicate as described in the “Microwell plate assay” section.

PCR and gel-electrophoretic genotyping: Colony PCR was conducted to confirm the genetic deletion of recombinase-mediated deletion circuits. Cells containing deletion circuits for recombinases A118, Bxb1, Int2, Int3, Int5, Int8, Int12, and TP901 (shown in FIGS. 5A-5C) transformed both with and without the corresponding recombinase-expression plasmids were precultured and assayed as described in the “Microwell plate assay” subsection. Colony PCR was performed directly from the assay plates following fluorescence reading; 1 μL of cells diluted in L of DI H₂O was used as template for these reactions. Primers were designed to bind upstream and downstream of the deletion region such that an undeleted circuit would generate a PCR product of ˜1,300 bp, and a deleted circuit would generate a PCR product of ˜200 bp (with slightly different spacing depending on the length of each recombinase's attachment sites). PCRs were carried out with a C1000 Touch Thermal Cycler (Bio-Rad) and preceded by a 10-minute time period at 98° C. to lyse cells. The PCR products were analyzed by electrophoresis on a 2.0% agarose gel. The sequences of the colony PCR primers are as follows: GenotypeF 62: CATCCAGTTTACTTTGCAGGG (SEQ ID NO: 2), GenotypeR 62: GATAACAAACTAGCAACACCAGAAC (SEQ ID NO: 3).

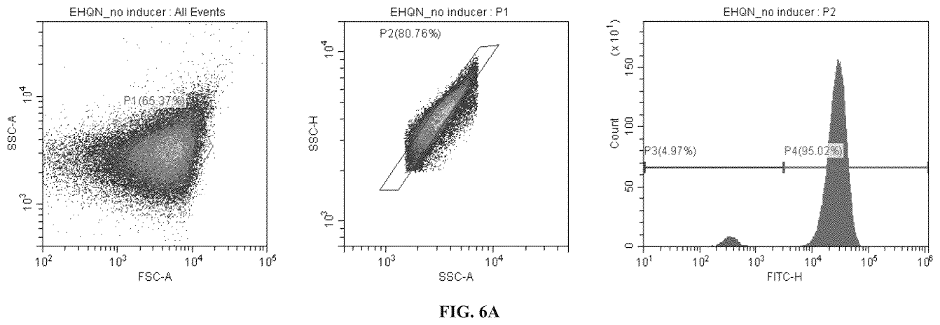

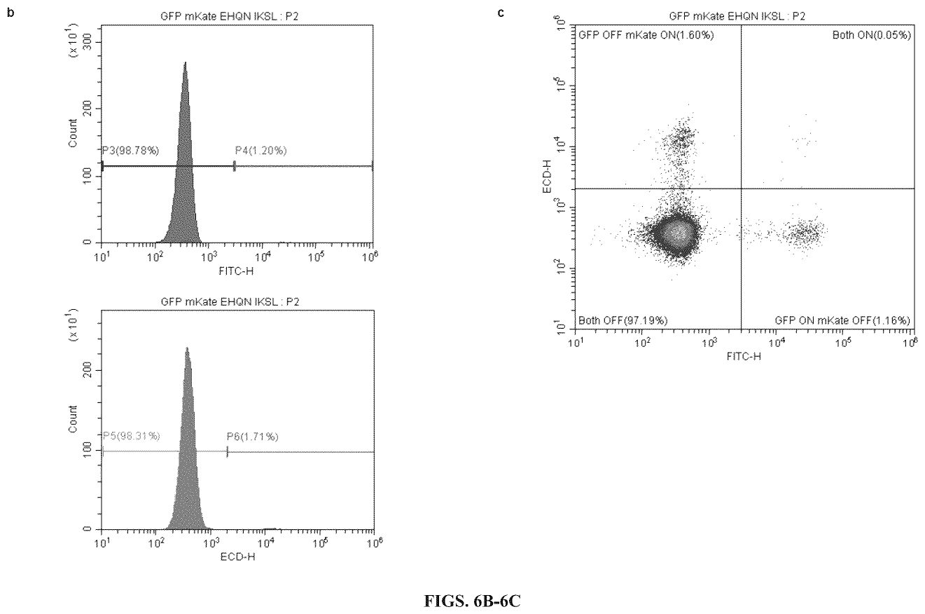

Flow cytometry: Fluorescence analysis was performed with a Beckman Coulter Cytoflex S flow cytometer. Cells were grown as described in “Microwell plate assay,” then passaged for an additional 24 hours with no inducer as described in “Recombinase three-day kinetic assays.” Cells were then diluted 1:19 into PBS with 2 mg/ml kanamycin and incubated for at least 1 hour at room temperature to inhibit further protein synthesis. Cells were processed at 10-L/min and monitored through the FITC channel for GFP expression and (where applicable) the ECD channel for mKate expression. Events were gated by forward scatter area vs. side scatter area to eliminate debris and then gated by side scatter height vs. side scatter area to discriminate doublets. More than 10,000 events were collected for final analysis. The cytometry gain and the gating procedure is shown in FIGS. 6A-6C.

### Example 2: Engineering a Deletion Circuit with Post-Translation Control—Defining a Next-Generation Memory Circuit

Recombinase attachment sites are sets of DNA elements (attB and attP), that can be partitioned—relative to a central conserved region—into 4 half-sites ˜25-35 bp each. When half-site DNA elements are combined, this results in full attachment sites on the order of ˜50-70 bp per attB or attP, forming a pseudopalindrome relative to the central motif, see FIGS. 2A-2B, FIGS. 7A-7H.

Structural information implies that the minimum unit for a given recombinase-DNA complex is 2 half-sites and 2 recombinases [29]. In addition, studies have demonstrated that in many cases, each half-site is tolerant to mutation [30], [31], [32]. Accordingly, the study posited that a given half-site could be omitted or modified and retain specific recombinase activity. To test this assertion, a deletion circuit was constructed in which the att sites corresponding to recombinase A118 are placed in the same orientation (i.e., aligned)—see FIG. 1C and FIG. 2A—flanking a reading-frame encoding green fluorescent protein (GFP) and a constitutive promoter contained within an Escherichia coli (E. coli) chassis cell, see FIG. 8A. Next, each of the half-sites (B1, B2, P1, P2) was omitted, and each circuit was exposed to the cognate A118 recombinase (see FIG. 8B, FIG. 7A). Congruent with the study's supposition, all four half-sites were amenable to omission as each variant resulted in reduced GFP fluorescence. Next, 7 additional omission circuits with attachment sites corresponding to recombinases TP901, Int2, Int3, Int12, Bxb1, Int5, and Int8 (see FIGS. 8C-8I) were built and tested. This experiment evaluated recombinase functions under moderate conditions—opposed to designing optimized circuits—accordingly, the promoter strength, RBS strength, and plasmid copy numbers were fixed. At the outset, the study was interested in evaluating recombinase functions under moderate conditions—opposed to designing optimized circuits—accordingly the promoter strength, RBS strength, and plasmid copy numbers were fixed. Namely, all recombinase plasmids (pSK001-pSK012) have a medium copy number p15 origin of replication, and the promoter and RBS upstream of each recombinase is fixed (see FIGS. 9A-9H). In all cases half-site omission was tolerated at 1 or more position(s) in each of the aforementioned systems—except in the case of Bxb1. Ghosh et al. demonstrated that the recombinase Bxb1 cannot recombine a partial attB site with an attP site based on a gel shift assay [33], accordingly Bxb1 can be regarded as a negative control.

In general, the study's supposition with regard to half-site omission was correct. Accordingly, the study posited that a given half-site could also support a substitution with a ˜16 bp DNA operator, see FIG. 1C—cognate to a given transcription factor that was engineered in a previous study [2], [3]. Correspondingly, the study supposed that the modified attachment site could facilitate post-translational regulation of recombinase function, see FIGS. 8J-8K. Given, that the A118 system displayed a high tolerance to half-site omission (FIG. 8B), a coarse-grained scan of operator substitution across the attP site was conducted without modification to the central conserved dinucleotide AA (see FIGS. 8J-8L). The justification for initially focusing on the attP attachment site for substitution with operator DNA—configured as a deletion (aligned) memory circuit—was predicated on this iteration of the memory circuit facilitating the evaluation of recombinase interception as a simple function, as opposed to a compound function that would involve gene regulation.

Given, that the A118 system displayed a high tolerance to half-site omission (FIG. 8A), a coarse-grained scan of operator substitution across the attP site was conducted, without modification to the central conserved dinucleotide AA. While recombinases may require identical central conserved sites in both the attB and attP for recombination, thus varying the central conserved region of the attP attachment site can generate a non-functional set. The justification for focusing on the attP attachment site was two-fold: (i) this design maintained deconvoluted regulation of the promoter, and (ii) this design facilitated independent recombinase interception. In the second criterion, placing an operator in proximity to the promoter could result in the regulation of the output gene via blocking the RNA polymerase. Accordingly, in this iteration of the synthetic memory circuit modification of the attachment site proximal to the promoter (i.e., attB) was avoided. In the scanning experiment given in FIG. 8L, the O^{ttg} operator was placed at 7 disparate positions (P−24, P−18, P−15, P−14, P+1, P+3, and P+4—i.e., without changing the central dinucleotide) and paired with the E⁺_{HQN} transcription factor, with constitutive A118 recombinase expression. The downstream numbering convention is based on the position of the first base pair of the operator relative to the first base pair of the central motif. Once the complete operator is beyond the central motif, the numbering is reset to 1 relative to the last base pair of the conserved region.

In the presented scanning experiment, the O^{ttg} operator was placed at 7 disparate positions (P−24, P−18, P−15, P−14, P+1, P+3, and P+4—i.e., without changing the central dinucleotide), and paired with the constitutive expression of the E⁺_{HQN} transcription factor and A118 recombinase. Notably, only 3 out of 7 positions supported interception of recombinase function, and maintenance of TF induction, under the conditions tested. Namely, operator positions P−18, P−15, and P+1 disrupted recombination in the presence of the E⁺_{HQN} transcription factor without inducer, and phenotypes were confirmed by complementary qualitative genotype experiments (see FIGS. 5A-5C). Upon the addition of the cellobiose ligand the TF was induced, and the attachment site was de-protected and allowed the recombinase to delete the GFP circuit. The putative mechanism for interception is given in FIG. 8K. The P+1 iteration of this circuit exhibited the best performance. These results allowed the study to glean a first design rule, inferring that the position of an operator can impact interception performance.

### Example 3: Engineering Next-Generation Memory Circuits Using Transcription Factors with Alternate DNA Binding

Given the high performance of the P+1 iteration of the interception memory circuit—composed of the A118 attP attachment site substituted with the O^{ttg} operator and of E⁺_{HQN} regulator—the study posited that the DNA operator element could be systematically varied at this fixed position (see FIG. 10A). Here the CelR (E⁺) regulatory core domain was maintained, while varying the DNA binding function using 5 additional alternate and orthogonal DNA operator elements (i.e., O^{gtg}, O^{gac}, O^{tta}, O^{ctt}, O^{agg}), see FIG. 10B. In this iteration of the experiment, the promoter strength, RBS strength, and plasmid copy numbers were fixed—as the study was initially interested in the discovery of recombinase function under moderate conditions, as opposed to designing an optimized circuit. In all cases (apart from the E⁺_{HTK}|O^{ctt} set), the said interception memory circuits with alternate TF binding were functional. Specifically, the substitution of Ottg with any of the following operators O^{gtg}, O^{gac}, O^{tta}, or O^{agg} and cognate TFs (i.e., E⁺_{YQR}, E⁺_{GKR}, E⁺_{TAN}, or E⁺_{KSL}—respectively) disrupted recombination in the absence of cellobiose—observed as an intact reading frame with GFP expressed. However, upon the addition of cellobiose, each E⁺_{ADR} transcription factor—with alternate DNA recognition (ADR)—was induced and the deletion of the GFP circuit was observed. Initially, the study posited that the E⁺_{HTK}|O^{ctt} at P+1 memory circuit fail due to low DNA binding affinity as both states with and without the ligand were deleted. Assessment of the general binding of each of the TF supports this initial supposition from the vantage-point of dynamic range and the extent of leakiness in the bound state—with few exceptions (see FIGS. 11A-11H). While the general performance of the E⁺_{HTK} transcription factor was the lowest in terms of dynamic range; however, the leakiness was on par with the E⁺_{TAN}|O^{tta} transcription factor. Accordingly, the study revised its supposition and posited that in addition to the transcription factor performance the composition of the attachment site may also impact the performance of an interception circuit. A sequence alignment of the substituted attP sites relative to the wild-type attachment site revealed that the attP with the O^{ctt} substitution and the attP with the O^{gac} substitution had the highest sequence similarity to wild-type attP (see FIGS. 11A-11H). This observation implies that the attP O^{ctt} substituted site—perhaps followed by the attP O^{gac} substituted site—is likely catalytically more efficient with respect to recombination relative to the other modified attP sites, rationalizing the observation for the performance of the E⁺_{HTK}|O^{ctt} interception circuit. Accordingly, the study articulated a second design rule, which purports that transcription factor DNA binding affinity impacts interception efficiency—and this property can be confounded by the variation of the sequence of the attP site, which could impact recombination catalytic efficiency. In addition to evaluating the general performance of regulated synthetic memory circuits, the study evaluated interception over 3 days. Namely, it evaluated 3 systems regulated by (i) E⁺_{HQN}|O^{ttg}, (ii) E⁺_{YQR}|O^{gtg}, and (iii) E⁺_{KSL}|O^{agg} as exemplars of the longitudinal stability of intercepted recombinase function. In all cases, as time increased the stability of the protected circuit was evidenced—see TABLE 11.

TABLE 11

| Transcription | Day 1 Dynamic | Day 2 Dynamic | Day 3 Dynamic |
|---|---|---|---|
| Factor | Range | Range | Range |
| E⁺_{HQN} | 20.63 | 87.93 | 114.83 |
| E⁺_{KSL} | 14.73 | 49.84 | 71.51 |
| E⁺_{YQR} | 20.91 | 85.96 | 141.52 |
| I⁺_{HQN} | 15.43 | 35.23 | 57.65 |
| I⁺_{KSL} | 5.44 | 6.04 | 7.42 |
| I⁺_{YQR} | 9.26 | 21.14 | 29.77 |

### Example 4: Engineering Next-Generation Memory Circuits with Expanded INPUT Processing Capability

Memory circuits with different engineered transcription factors have been designed, built, and tested to facilitate expanded INPUT processing (see FIGS. 10C-10F). Initially, the operator position was fixed (i.e., P+1 with respect to the A118 attP attachment site), and the composition of the operator substitution was fixed (i.e., O^{gtg} DNA element—cognate to the YQR binding domain), while varying the regulatory core domain of the TF to enable processing of 4 additional inputs—i.e., I⁺_{YQR} (IPTG), R⁺_{YQR} (ribose), F⁺_{YQR} (fructose), G⁺_{YQR} (fucose). Qualitatively, all 4 memory circuits performed as expected such that un-induced systems reduced circuit deletion, while induced TFs with cognate ligands de-protected the attP site and resulted in circuit deletion. The study posited that the quantitative differences in deletion between circuits could be attributed to differences in TF-operator affinity—congruent with the second design rule (see FIGS. 11A-11H).

In the next iteration of the memory circuits with expanded INPUT processing, the study varied the DNA operator for each of the given TFs (i.e., O^{gac}, O^{tta}, O^{ttg}, O^{ctt}, and O^{agg}—cognate to ADR domains GKR, TAN, HQN, HTK, and KSL respectively) and evaluated interception. In summary, putative interception memory circuits now contain two variables: (i) the TF and (ii) the DNA operator element (see FIGS. 10C-10F). Congruent with the A118 attachment sites substituted at position P+1 with alternate operators and cognate E⁺_{ADR} regulators (FIG. 10B), the majority of said interception memory circuits with alternate TF and operator binding were functional—except for HTK|O^{ctt} (in all cases). Moreover, the attP O^{gac} substituted site was the second least effective memory circuit even when the general binding metrics would imply a reasonable probability for interception (e.g., I⁺_{GKR}|O^{gac}, and R⁺_{GKR}|O^{gac}—see FIGS. 11A-11H). This observation is consistent with expectation given the purported differences in catalytic efficiency for the attP O^{ctt} substituted and attP O^{gac} substituted attachment sites based on the sequence alignment—even after accounting for the poor binding performance of TFs from the HTK and GKR sets (also see FIGS. 11A-11H). To affirm interception memory at the population level the study used flow cytometry for a subset of best-performing operations, see FIGS. 11A-11H. In general, the study demonstrated that: (i) interception was capable of protecting the attachment site—preventing recombination—with near perfection in many cases, and (ii) deprotected memory circuits resulted in recombination for the entire population of unrecombined cells, in nearly every case. In general, all of the tested interception memory circuits performed according to the supposition posited by the second design rule. Namely, the (i) performance metrics for each TF and (ii) qualitative catalytic efficiency of modified attachment sites dictate the performance of a given synthetic (interception) memory circuit. However, (iii) increased catalytic efficiency of a given attP site can be reduced with sufficient binding function of the TF.

The (i) performance metrics for each TF and (ii) qualitative catalytic efficiency of modified attachment sites dictate the performance of a given synthetic (interception) memory circuit. However, (iii) the increased catalytic efficiency of a given attP site can be mitigated with sufficient binding function of the TF. The (iii) criteria are predicated on the observations made for the GKR|attP-O^{gac}, set in FIGS. 10B-10F. (also see FIGS. 11A-11H). Namely, while the memory circuits that utilize the R⁺_{GKR}, F⁺_{GKR}, G⁺_{GKR}, and I⁺_{GKR} transcription factors all exhibit low interception performance, the E⁺_{GKR} iteration displays relatively strong interception performance. Correspondingly, the E⁺_{GKR} has the best performance metrics in the GKR class of engineered TFs.

To demonstrate that interception memory circuits can be tuned to improve performance three variants were selected from the RbsR set with the poorest performance—i.e., (i) R⁺_{GKR}|O^{gac}, (ii) R⁺_{TAN}|O^{tta}, and (iii) R⁺_{HTK}|O^{ctt}. While all three operator substituted attP sites had the highest sequence similarity to the wild-type attachment site (see FIGS. 11A-11H) the study posited that the circuit performance could be improved via diminishing the apparent recombinase (A118) activity. To accomplish this, the study reduced the RBS strength cognate to A118 production, effectively reducing the amount of catalyst available for recombination—see FIG. 12. In all cases, a marked improvement in circuit performance was observed for all three iterations of synthetic memory circuits—affirming the study's supposition.

### Example 5: Engineering Permissive Interception Memory Circuits—INPUT Processing Via Anti-Repression

A collection of anti-repressors, e.g., anti-LacI (I^{A}) [2], [34], anti-RbsR (R^{A}) [2], anti-FurR (F^{A}) [2], anti-GalS (S^{A}) [35], and PurR (P^{A}) [36], were recently engineered that are phenotypically antithetical (see FIG. 1E) to many of the repressors used in FIGS. 10A-10F. Namely, in the context of gene regulation (e.g., with GFP as the OUTPUT) repressors function as BUFFER operations, whereas anti-repressors function as NOT operations—descriptions given in FIG. 1A and FIG. 1E. Accordingly, the anti-repressor phenotype can be described as permissive in that DNA binding is only permitted in the presents of the INPUT ligand. The study posited that permissive interception memory operations that could retain the reading frame of a given circuit only if the cognate exogenous signal was present could be designed, built, and tested—a general description given in FIG. 13A. Initially, deletion circuits were constructed with the O^{ttg} operator substituted within the A118 attP half-site at the P+1 position, and the circuit was paired with one of the given anti-repressors—i.e., I^{A}_{HQN}|O^{ttg} (FIG. 1B), R^{A}_{HQN}|O^{ttg} (FIG. 1C), F^{A}_{HQN}|O^{ttg} (FIG. 1D), S^{A}_{HQN}|O^{ttg} (FIG. 1E), and P^{A}_{HQN}|O^{ttg} (FIG. 1F). In the absence of ligand, all of the tested anti-repressors allowed the cognate recombinase to delete the circuit (see FIGS. 13A-13F and FIGS. 14A-14E). However, in the presence of the cognate ligands—i.e., IPTG (I^{A}_{HQN}|O^{ttg}), ribose (R^{A}_{HQN}|O^{ttg}), fructose (F^{A}_{HQN}|O^{ttg}), fucose (S^{A}_{HQN}|O^{ttg}), and hypoxanthine (P^{A}_{HQN}|O^{ttg})—memory circuits exhibited induced protection (interception) observed as maintenance of the GFP circuit. To demonstrate generalizability, the O^{ttg} operator was replaced with five additional operators—i.e., O^{gac}, O^{tta}, O^{gtg}, O^{ctt}, O^{agg}, corresponding to binding motifs GKR, TAN, YQR, HTK, and KSL respectively. Note, this is the same set of DNA binding functions used for the repressors tested in FIGS. 10A-10F. In addition to synthetic transcription factors X^{A}_{HQN} (where X=I, R, F, S or P) X^{A}_{YQR} and X^{A}_{KSL} facilitated some degree of permissive protection of the deletion circuit—i.e., in the presents of the given cognate ligands. Moreover, similar to the engineered repressors adapted with the HTK binding motif (i.e., X⁺_{HTK} where X=I, R, F, G or E—see FIGS. 10A-10F) used for type II memory, all engineered X^{A}_{HTK} anti-repressors with the said DNA binding function failed to protect the deletion circuit, see FIGS. 13A-13F. However, in addition to X⁺_{HTK}, anti-repressors adapted with GKR (X^{A}_{GKR}) also failed to permissively protect the deletion circuit. Based on these observations, the study concluded that the permissive maintenance of a deletion circuit via interception is possible. However, given the mechanism of protection (i.e., expression and folding of the TF precedes ligand binding—which is required for interaction with the substituted operator at the attachment site), fewer successful operations were observed. Notably, decreasing the RBS strength to produce less recombinase did not necessarily improve interception synthetic memory (see FIG. 15). Rather, tuning the RBS resulted in overprotected circuits—at best. Accordingly, this observation implies that improving the performance of permissive synthetic memory circuits would likely require additional protein engineering or pre-conditioning, as opposed to circuit optimization. Finally, inducible and permissive memory can be brought together with nested logical operations to form systems capable of decision-making and memory operations—see FIGS. 16A-16J, FIGS. 17A-17D.

### Example 6: Engineering Next-Generation Memory Circuits with Orthogonal Recombinase Functions

The studies described herein have only focused on the development of interception memory circuits using the A118 recombinase and cognate attachment sites. Noting the general performances of half-site omissions for other recombinases (FIGS. 8A-8L) and the A118 exemplars given in FIGS. 10A-10F and FIGS. 13A-13F, a study was conducted which posited that P+1 iterations of interception memory circuit responsive to additional recombinase functions could successfully be constructed—in the context of a deletion memory circuit (general design given in FIG. 18A). First, 7 new circuits were designed, built and tested with attachment sites that corresponded to recombinases TP901, Int2, Int3, Int12, Bxb1, Int5 and Int8 (see FIGS. 18C-18I—gray boxes). For this set of circuit designs, the DNA operator O^{ttg} (cognate to E⁺_{HQN}) was substituted at the P+1 position within the attP site for each of the 7 additional recombinases (illustrated in FIG. 18A). The rationale for selecting the O^{ttg} operator (cognate to the E⁺_{HQN} transcription factor) and the P+1 position was that this iteration of the interception memory circuit had one of the highest performances when tested with the A118 recombinase (see FIGS. 8A-8L, FIGS. 10A-10F, and FIG. 18B). Next, each of the P+1 deletion circuits were evaluated with and without the corresponding recombinase (i.e., unregulated), see FIGS. 18A-18I—gray boxes. Briefly, 5 out of the 7 additional circuits resulted in deletion upon the concurrent production of the cognate recombinase—i.e., excluding Bxb1 and Int2. As expected, the Bxb1 P+1 circuit showed abrogated function upon modification, congruent with the results shown in FIG. 8H. In the case of the Int2 deletion circuit, the study posited that given the performance of the P2 half-site omission (see FIG. 8I), the operator substitution at the P+1 position compromised the recombinase function.

Next, the corresponding regulated memory circuits were constructed with the E⁺_{HQN} transcription factor present, and concurrent expression of a given recombinase (cognate to the attachment site). Briefly, the regulated memory circuits with demonstrated recombinase function displayed the correct qualitative performances (see FIGS. 18A-181—red outlined boxes). Namely, the transcription factor E⁺_{HQN} intercepted recombinase-mediated deletion in all 5 functional circuits—i.e., TP901, Int3, Int12, Int5 and Int8. Upon induction with cellobiose a given circuit was de-protected, and deletion ensued for said functional circuits. Finally, variation in the position of the DNA operator within the attP site was tested (see FIGS. 19A-19F). In general, interception was observed in at least one additional site for at least 5 of the functional systems, based on a coarse-grained scan. However, none of the previously non-functional systems (i.e., Bxb1 and Int2) exhibited recovery. From this set of experiments, it was gleaned that: (i) in general variation of the attachment site is tolerated, (ii) however, TF binding can confound interception outcomes, (iii) and multiple operator positions can be supported for a given attP site and correlated with tolerance to general half-site omission.

### Example 7: Interception with Combinational (2-INPUT) Information Processing

The system of synthetic transcription factors was originally developed to work in collaboration, forming 2-INPUT gene control (decision-making) from fundamental single input operations—facilitated via directing two or more non-synonymous transcription factors to the same DNA element [1], [2], [3]. Likewise, the study posited that 2-INPUT interception memory circuits could be built using similar design principles (see FIG. 3A). In the first iteration of a 2-INPUT memory circuit, the E⁺_{YQR} and I⁺_{YQR} repressors were paired via the O^{gtg} operator element (see FIG. 3A-3B). Congruent with the design goal, the circuit remained intact without ligand or only with one ligand present. Deletion of the circuit ensued only when the system was exposed to both signals concurrently. To demonstrate the generalizability of 2-INPUT interception memory four additional iterations were constructed with variation and ligand response and DNA binding function, see FIGS. 3C-3F. Next, an antithetical interception memory operation was constructed using two anti-repressors that processed disparate inputs, with synonymous DNA binding functions, see FIG. 3G. In the said memory system, deletion was only possible in the absence of both input signals. Finally, a mixed interception operation was constructed in which a repressor was paired with an anti-repressor, see FIG. 3H. In this iteration, deletion of the circuit was only possible in the presence of cellobiose and was intercepted in all other cases.

### Example 8: Interception Synthetic Memory with Inversion Addresses

The study posited that an iteration of interception could be designed in which the attachment site configuration would facilitate inversion (opposed to deletion) upon induction of a cognate repressor. This iteration of interception memory required the anti-alignment of attachment sites; see general designs given in FIG. 2B. The envisioned inversion memory circuit would facilitate regulated and inheritable gain-of-function, in contrast to the regulated loss-of-function facilitated by demonstrated deletion interception circuits. Here the design required that the placement of the DNA operator is taken into consideration to prevent canonical gene regulation. In the first iteration, the operator within the attP site (cognate to the A118 recombinase) was substituted at the P+1 position, such that upon inversion, the operator is distal (far upstream) to the promoter (see FIG. 20A). Noting that final placement of the operator proximal (downstream) of the promoter would likely result in regulation (i.e., blocking in the uninduced state) of RNA polymerase readthrough. Congruent with the circuit design, A118 recombinase activity was intercepted in the absence of a ligand (i.e., the promoter remained inverted in the presence of recombinase). Upon the induction of the I⁺_{HQN} transcription factor the attachment site becomes deprotected, and the promoter was inverted, facilitating the production of GFP. To demonstrate the generalizability of the design, three additional iterations of inversion interception circuits for different recombinases—i.e., Int3 (FIG. 20B), Int8 (FIG. 20C), and Int12 (FIG. 20D)—were built and tested with variation in operator placement or attachment site configuration. All tested inversion interception circuits performed as expected. Moreover, upon the removal of the ligand the inverted circuit maintained the ON-state verifying that the transcription factor was divorced from gene regulation.

### Example 9: Expanding Synthetic Memory Capacity

In addition to the above, a study was conducted which posited that interception synthetic memory could be expanded via modifying the central motif of a given set of attachment sites. This supposition is predicated on pairs of attachment sites that share the same central motif can recombine, whereas mismatched central motifs cannot recombine [17], [37]. In other words, recombinases require identical central conserved sites for recombination, thus varying the central conserved region of the attachment sites purportedly generates orthogonal attachment site pairs. The study posited that the central motif could be varied (to generate orthogonal attachment sites) and an operator within the attP concurrently substituted at position P+1 (to facilitate interception). Combining attachment site orthogonality and non-synonymous interception—in principle—can facilitate the systematic expansion of this iteration of synthetic memory. To test this assertion all 6 putatively orthogonal variations to the central motif of attachment site A118 pairs designed, built, and tested with an O^{ttg} operator at position P+1 (see FIG. 21). Upon testing each of the deletion memory circuits and corresponding mismatches the study affirmed its supposition that interception can be paired with variation in the central motif.

Next, memory circuits were tested with (i) variation in the central dinucleotide for A118 attachment sites paired with (ii) variation in the substituted operator DNA (see FIG. 21 and FIGS. 22A-22F). Here it was illustrated that paired orthogonal DNA elements—i.e., orthogonal A118 attachment sites+orthogonal operators—and disparate transcription factors could regulate interception in putatively orthogonal memory circuits. In turn, this analysis was used to identify the two best performing sets of putatively orthogonal fundamental interception memory operations (see FIG. 21). The testing and analysis revealed that the (i) E⁺_{HQN}|attP A118-CA and (ii) I⁺_{KSL}|attP A118-AA single input circuits had outstanding performance and were putatively orthogonal in terms of input signal and attachment site recombination. The study posited that using this set of fundamental memory operations a 2-OUTPUT interception memory circuit could be constructed such that E⁺_{HQN}|attP A118-CA corresponded to mKate regulated deletion and I⁺_{KSL}|attP A118-AA corresponded to GFP regulated deletion (see FIG. 21). The purpose of this memory circuit was to demonstrate that interception orthogonality was possible with a single recombinase facilitated by orthogonal sets of attachment sites. Congruent with the study's supposition it was demonstrated that each deletion occurred independently and was not confounded by unintended putative states—e.g., inversions or off-target deletions (see FIG. 21 and complementary flow cytometry data given in FIGS. 23A-23B and FIGS. 24A-24G).

### Example 10: Synthetic Memory Kinetics

An experiment was conducted that used the same set of parts to construct type-I and type-II memory circuits (i.e., identical promoters, insulators, RBS, and GFP OUTPUT) to maintain consistent gene expression and putative burden on the chassis cell. The only difference between the memory circuits is the design. Namely, for type-I memory the E⁺_{YQR} transcription factor regulated the expression of the recombinase, whereas the E⁺_{YQR} transcription factor regulated the recombination event directly (post-translation) in type-II memory, FIGS. 22C-22D and FIGS. 24A-24G. In all cases interception memory was significantly faster than the corresponding canonical (type-I) design—i.e., occurring nearly instantaneously opposed to hours or days. The increased rate of type-II memory over type-I memory was attributed to: (i) the maintenance of high levels of mature recombinase at steady-state, which (ii) leads to near instantaneous binding to the attB site. Moreover, the TF and recombinase are in a dynamic equilibrium at the substituted attP site. Once the TF (E⁺_{YQR}) is induced, (iii) A118 recombinase binding and subsequent recombination occur near instantaneously.

### Discussion

Exemplars of decision-making have been demonstrated as Boolean logical operations [1]-[6]. Synthetic memory has been demonstrated in myriad ways, e.g., bistable toggle switches [7]-[9], CRISPR-based editing [10], and recombinase facilitated DNA rearrangements [11]-[17]. Memory operations can also mediated via a subclass of recombinases collectively identified as large serine integrases [18]. Notably, this iteration of memory imparts permanent genetic changes and can be programmed to achieve both gain-of-function (GOF) and loss-of-function (LOF).

The following patents, applications and publications as listed below and throughout this document are hereby incorporated by reference in their entirety herein.

### REFERENCE LIST

[1] Huang, B. D., Groseclose, T. M. & Wilson, C. J. Transcriptional programming in a Bacteroides consortium. Nat Commun 13, 3901 (2022).
[2] Groseclose, T. M., Rondon, R. E., Herde, Z. D., Aldrete, C. A. & Wilson, C. J. Engineered systems of inducible anti-repressors for the next generation of biological programming. Nat Commun 11, 4440 (2020).
[3] Rondon, R. E., Groseclose, T. M., Short, A. E. & Wilson, C. J. Transcriptional programming using engineered systems of transcription factors and genetic architectures. Nat Commun 10, 4784 (2019).
[4] Nielsen, A. A. et al. Genetic circuit design automation. Science 352, aac7341 (2016).
[5] Taketani, M. et al. Genetic circuit design automation for the gut resident species Bacteroides thetaiotaomicron. Nat Biotechnol 38, 962-969 (2020).
[6] Milner, P. T. et al. Performance Prediction of Fundamental Transcriptional Programs. ACS Synth Biol (2023).
[7] Kobayashi, H. et al. Programmable cells: Interfacing natural and engineered gene networks. Proceedings of the National Academy of Sciences of the United States of America 101, 8414-8419 (2004).
[8] Burrill, D. R. & Silver, P. A. Making Cellular Memories. Cell 140, 13-18 (2010).
[9] Becskei, A., Seraphin, B. & Serrano, L. Positive feedback in eukaryotic gene networks: cell differentiation by graded to binary response conversion. Embo Journal 20, 2528-2535 (2001).
[10] Nuñez, J. K. et al. Genome-wide programmable transcriptional memory by CRISPR-based epigenome editing. Cell (2021).
[11] Guo, L. et al. Engineering Escherichia coli lifespan for enhancing chemical production. Nature Catalysis 3, 307-318 (2020).
[12] Yang, L. et al. Permanent genetic memory with >1-byte capacity. Nat Methods 11, 1261-1266 (2014).
[13] Ghosh, P., Kim, A. I. & Hatfull, G. F. The orientation of mycobacteriophage Bxb1 integration is solely dependent on the central dinucleotide of attP and attB. Mol Cell 12, 1101-1111 (2003).
[14] Loessner, M. J., Inman, R. B., Lauer, P. & Calendar, R. Complete nucleotide sequence, molecular analysis and genome structure of bacteriophage A118 of Listeria monocytogenes: implications for phage evolution. Mol Microbiol 35, 324-340 (2000).
[15] Zúñiga, A. et al. Rational programming of history-dependent logic in cellular populations. Nat Commun 11, 4758 (2020).
[16] Ham, T. S., Lee, S. K., Keasling, J. D. & Arkin, A. P. Design and construction of a double inversion recombination switch for heritable sequential genetic memory. PLoS One 3, e2815 (2008).
[17] Smith, M. C. A., Till, R. & Smith, M. C. M. Switching the polarity of a bacteriophage integration system. Molecular Microbiology 51, 1719-1728 (2004).
[18] Merrick, C. A., Zhao, J. & Rosser, S. J. Serine Integrases: Advancing Synthetic Biology. ACS Synth Biol 7, 299-310 (2018).
[19] Lapique, N. & Benenson, Y. Digital switching in a biosensor circuit via programmable timing of gene availability. Nat Chem Biol 10, 1020-1027 (2014).
[20] Durrant, M. G. et al. Systematic discovery of recombinases for efficient integration of large DNA sequences into the human genome. Nat Biotechnol 41, 488-499 (2023).
[21] Siuti, P., Yazbek, J. & Lu, T. K. Synthetic circuits integrating logic and memory in living cells. Nat Biotechnol 31, 448-452 (2013).
[22] Akboga, D., Saltepe, B., Bozkurt, E. U. & Seker, U. O. S. A Recombinase-Based Genetic Circuit for Heavy Metal Monitoring. Biosensors (Basel) 12 (2022).
[23] Bessen, J. L. et al. High-resolution specificity profiling and off-target prediction for site-specific DNA recombinases. Nat Commun 10, 1937 (2019).
[24] Meinhardt, S. et al. Novel insights from hybrid LacI/GalR proteins: family-wide functional attributes and biologically significant variation in transcription repression. Nucleic Acids Research 40, 11139-11154 (2012).
[25] Milk, L., Daber, R. & Lewis, M. Functional rules for lac repressor-operator associations and implications for protein-DNA interactions. Protein Science 19, 1162-1172 (2010).
[26] Zhang, Q., Azarin, S. M. & Sarkar, C. A. Model-guided engineering of DNA sequences with predictable site-specific recombination rates. Nature Communications 13, 4152 (2022).
[27] Weinberg, B. H. et al. High-performance chemical- and light-inducible recombinases in mammalian cells and mice. Nature Communications 10 (2019).
[28] Landry, B. P. & Tabor, J. J. Engineering Diagnostic and Therapeutic Gut Bacteria. Microbiology Spectrum 5 (2017).
[29] Ghosh, P., Pannunzio, N. R. & Hatfull, G. F. Synapsis in phage Bxb1 integration: selection mechanism for the correct pair of recombination sites. J Mol Biol 349, 331-348 (2005).
[30] Li, H., Sharp, R., Rutherford, K., Gupta, K. & Van Duyne, G. D. Serine Integrase attP Binding and Specificity. J Mol Biol 430, 4401-4418 (2018).
[31] Gupta, M., Till, R. & Smith, M. C. Sequences in attB that affect the ability of phiC31 integrase to synapse and to activate DNA cleavage. Nucleic Acids Res 35, 3407-3419 (2007).
[32] Singh, S., Ghosh, P. & Hatfull, G. F. Attachment site selection and identity in Bxb1 serine integrase-mediated site-specific recombination. PLoS Genet 9, e1003490 (2013).
[33] Ghosh, P., Pannunzio, N. R. & Hatfull, G. F. Synapsis in Phage Bxb1 Integration: Selection Mechanism for the Correct Pair of Recombination Sites. Journal of Molecular Biology 349, 331-348 (2005).
[34] Richards, D. H., Meyer, S. & Wilson, C. J. Fourteen Ways to Reroute Cooperative Communication in the Lactose Repressor: Engineering Regulatory Proteins with Alternate Repressive Functions. Acs Synthetic Biology 6, 6-12 (2017).
[35] Groseclose, T. M., Hersey, A. N., Huang, B. D., Realff, M. J. & Wilson, C. J. Biological signal processing filters via engineering allosteric transcription factors. Proc Natl Acad Sci USA 118 (2021).
[36] Rondon, R. & Wilson, C. J. Engineering Alternate Ligand Recognition in the PurR Topology: A System of Novel Caffeine Biosensing Transcriptional Antirepressors. Acs Synthetic Biology 10, 552-565 (2021).
[37] Colloms, S. D. et al. Rapid metabolic pathway assembly and modification using serine integrase site-specific recombination. Nucleic Acids Research 42 (2014).
[38] Reis, A. C. & Salis, H. M. An Automated Model Test System for Systematic Development and Improvement of Gene Expression Models. Acs Synthetic Biology 9, 3145-3156 (2020).
[39] Farasat, I. et al. Efficient search, mapping, and optimization of multi-protein genetic systems in diverse bacteria. Molecular Systems Biology 10 (2014).
[40] Patrick, W. M., Firth, A. E. & Blackburn, J. M. User-friendly algorithms for estimating completeness and diversity in randomized protein-encoding libraries Wayne M. Patrick and Andrew E. Firth contributed equally to this work. Protein Engineering, Design and Selection 16, 451-457 (2003).

SEQUENCES

| Sequence | SEQ ID NO |
|---|---|
| 5′-AATT N⁶N⁵N⁴ AGCGCT N′N′N′ AATT-3′ | 1 |
| CATCCAGTTTACTTTGCAGGG | 2 |
| GATAACAAACTAGCAACACCAGAAC | 3 |
| AAACGCAAAGAGGGAACTAAACACTT | 4 |
| AACTTTTCGGATCAAGCTATGAAAAACGCAAAGAGGGAACTAAACACTT | 5 |
| TTAGTTCCTCGTTTTCTCTCGTTAAAAGAAGAAGAAACGAGAAACTAAA | 6 |
| 3′-TTGAAAAGCCTAGTTCGATACTTTTTGCGTTTCTCCCTTGATTTGTGAA-5′ | 7 |
| 5′-TTTAGTTTCTCGTTTCTTCTTCTTTTAACGAGAGAAAACGAGGAACTAA-3′ | 8 |
| CGACGTCCAACATATGGCACCGG | 9 |
| GGAGGCTTTCGTCTTTTGACAGCT | 10 |
| CTGAGTAGGACAAATCCGCCGCC | 11 |
| CTAGACCTAGGGTACGGGTTTTGC | 12 |
| ATGCCAACACAATTAACATCTCAATCAAGGTAAATGCTTTTTGCTTTTTTTG | 13 |
| C | |
| TGGCAATTCCGACGTCCAAC | 14 |
| GGAGGCTTTCGTCTTTTGACAGCTAGCTCAG | 15 |
| AGCGAGTTTTTATTTCGTTTATTTCAATTAAGGTAACTAAAAAACTCCTTT | 16 |
| ACGCTCTCCTGAGTAGGACAAAT | 17 |
| CTAGACCTAGGGTACGGGTTTTGCTG | 18 |
| GTTTGTAAAGGAGACTGATAATGGCATGTACAACTATACTCGTCGGTAAAA | 19 |
| AGGCATCTTAT | |
| AATTCCGACGTCCAACATATGGCACCGGGTT | 20 |
| TTAACGGAGGCTTTCTCTTTTG | 21 |
| ATGGATAAAAAAATACAGCGTTTTTCATGTACAACTATACTAGTTGTAGTGC | 22 |
| CTAAATAATGCTT | |
| CTCTCCTGAGTAGGACAAATCCGCCGCCTTTT | 23 |
| TAACTAGACCTAGGGTACGGGTTT | 24 |
| GAGCGCCGGATCAGGGAGTGGACGGCCTGGGAGCGCTACACGCTGTGGCTG | 25 |
| CGGTCGGTGC | |
| TGCAGGGCTTCCCAACCTTACCAGA | 26 |
| TTGGAGGCTTTCGTCTTTTGACAGCT | 27 |
| CCCTAATACGCAAGTCGATAACTCTCCTGGGAGCGTTGACAACTTGCGCACC | 28 |
| CTGATCTG | |
| TCCTGAGTAGGACAAATCCGCCGCC | 29 |
| CTAGACCTAGGGTACGGGTTTTGCT | 30 |
| CAATCATCAGATAACTATGGCGGCACGTGCATTAACCACGGTTGTATCCCGT | 31 |
| CTAAAGTACTCGT | |
| GCAATTCCGACGTCCAACATATGGCACCGGGTT | 32 |
| TTAACGGAGGCTTTCGTCTTTTGACAGCTA | 33 |
| TTAATAAACTATGGAAGTATGTACAGTCTTGCAATGTTGAGTGAACAAACTT | 34 |
| CCATAATAAAAT | |
| TAACTAGACCTAGGGTACGGGTTTTGCTGC | 35 |
| GTTCGTGGTAACTATGGGTGGTACAGGTGCCACATTAGTTGTACCATTTATG | 36 |
| TTTATGTGGTTAAC | |
| GGCAATTCCGACGTCCAACATATGGCACCGG | 37 |
| GGAGGCTTTCGTCTTTTGACAGCTAGCTCAGTC | 38 |
| TTTTTGTATGTTAGTTGTGTCACTGGGTAGACCTAAATAGTGACACAACTGC | 39 |
| TATTAAAATTTAA | |
| CGCTCTCCTGAGTAGGACAAATCCGCCGCC | 40 |
| CTAGACCTAGGGTACGGGTTTTGCTGCCCGCAA | 41 |
| CGGCCGGCTTGTCGACGACGGCGGTCTCCGTCGTCAGGATCATCCGGGC | 42 |
| GGCTTCCCAACCTTACCAGAGGG | 43 |
| GTCGTGGTTTGTCTGGTCAACCACCGCGGTCTCAGTGGTGTACGGTACAAAC | 44 |
| CCCGAC | |
| CTCTCCTGAGTAGGACAAATCCGCCGCC | 45 |
| CTGTTCTGGTGTTGCTAGTTTGTTATCA | 46 |
| GGACGGCGCAGAAGGGGAGTAGCTCTTCGCCGGACCGTCGACATACTGCTC | 47 |
| AGCTCGTC | |
| CGACGTCCAACATATGGCACCGGGTT | 48 |
| TTAACGGAGGCTTTCGTCTTTTGACAGC | 49 |
| GCTCATGTATGTGTCTACGCGAGATTCTCGCCCGAGAACTTCTGCAAGGCAC | 50 |
| TGCTCTTGGCT | |
| CTGAGTAGGACAAATCCGCCGCCTTTT | 51 |
| TAACTAGACCTAGGGTACGGGTTTTGCTGCC | 52 |
| TTTTGAGCGCTCAAAA | 53 |
| TTAGTTCCTCGTTTTCTCTCGTTAATTTTGAGCGCTCAAAAAAACTAAA | 54 |
| TTTTGAGCGCTCAAAATCTCGTTAAAAGAAGAAGAAACGAGAAACTAAA | 55 |
| TTAGTTTTTTGAGCGCTCAAAATAAAAGAAGAAGAAACGAGAAACTAAA | 56 |
| TTAGTTCCTTTTTGAGCGCTCAAAAAAGAAGAAGAAACGAGAAACTAAA | 57 |
| TTAGTTCCTCTTTTGAGCGCTCAAAAAGAAGAAGAAACGAGAAACTAAA | 58 |
| TTAGTTCCTCGTTTTCTCTCGTTAAAATTTTGAGCGCTCAAAAACTAAA | 59 |
| TTAGTTCCTCGTTTTCTCTCGTTAAAAGTTTTGAGCGCTCAAAACTAAA | 60 |
| ATTXYZAGCGGATAACAAT | 61 |
| AEYAGVSADTVSRVVNQASHV | 62 |
| AATTXYZAGCGGATAACAATT | 63 |
| AAAGAGGAGAAA | 64 |
| TTAGTTCCTCGTTTTCTCTCGTTAATTCTTAGCGCTAAGAAAAACTAAA | 65 |
| TTAGTTCCTCGTTTTCTCTCGTTAATTGACAGCGCTGTCAAAAACTAAA | 66 |
| TTAGTTCCTCGTTTTCTCTCGTTAATTAGGAGCGCTCCTAAAAACTAAA | 67 |
| TTAGTTCCTCGTTTTCTCTCGTTAATTTTAAGCGCTTAAAAAAACTAAA | 68 |
| TTAGTTCCTCGTTTTCTCTCGTTAATTGTGAGCGCTCACAAAAACTAAA | 69 |
| ATTGTGAGCGCTCACAATT | 70 |
| AEYAGVSYQTVSRVVNQASHV | 71 |
| ATTGACAGCGCTGTCAATT | 72 |
| AEYAGVSGKTVSRVVNQASHV | 73 |
| ATTTTAAGCGCTTAAAATT | 74 |
| AEYAGVSTATVSNVVNQASHV | 75 |
| ATTTTGAGCGCTCAAAATT | 76 |
| AEYAGVSHQTVSNVVNQASHV | 77 |
| ATTCTTAGCGCTAAGAATT | 78 |
| AEYAGVSHTTVSKVVNQASHV | 79 |
| ATTAGGAGCGCTCCTAATT | 80 |
| AEYAGVSKSTVSLVVNQASHV | 81 |
| GCTCATGTATGTGTCTACGCGAGATTCTCGCCTTGTGAGCGCTCACAAGCAC | 82 |
| TGCTCTTGGCT | |
| CCCTAATACGCAAGTCGATAACTCTCCTGGGAGCGTTTTGAGCGCTCAAAAC | 83 |
| CTGATCTG | |
| TTTTTGTATGTTAGTTGTGTCACTGGGTAGACTTGTGAGCGCTCACAACTGC | 84 |
| TATTAAAATTTAA | |
| GTCGTGGTTTGTCTGGTCAACCACCGCGGTTTTTGAGCGCTCAAAAACAAAC | 85 |
| CCCGAC | |
| AGCGAGTTTTTATTTCGTTTATTTCTTTTGAGCGCTCAAAAAAACTCCTTT | 86 |
| GTTTGTAAAGGAGACTGATAATGGCATGTACAACTATACTTTGTGAGCGCTC | 87 |
| ACAATCTTAT | |
| ATGGATAAAAAAATACAGCGTTTTTCATGTACAACTATACTTTGTGAGCGCT | 88 |
| CACAATAATGCTT | |
| TTAATAAACTATGGAAGTATGTACAGTCTTGCAATTGTGAGCGCTCACAATT | 89 |
| CCATAATAAAAT | |
| TTAATAAACTATGGAATTGTGAGCGCTCACAAAATGTTGAGTGAACAAACT | 90 |
| TCCATAATAAAAT | |
| AEYAGVSFKTVSRVVNQASHV | 91 |
| AGTTTTGAGCGCTCAAAATTATTTCAATTAAGGTAACTAAAAAACTCCTTT | 92 |
| AGCGAGTTTTTGAGCGCTCAAAATCAATTAAGGTAACTAAAAAACTCCTTT | 93 |
| AGCGAGTTTTTATTTCGTTTATTTCAATTTTGAGCGCTCAAAAACTCCTTT | 94 |
| ATGGATAAAAAAATACAGCGTTTTTCATGTACAACTATACTATTTTGAGCGC | 95 |
| TCAAAAAATGCTT | |
| ATGGATAAAAAAATACAGCGTTTTTCATGTACAACTATACTAGTTTTGAGCG | 96 |
| CTCAAAAATGCTT | |
| ATGGATAAAAAAATACAGCGTTTTTCATGTACAACTATACTAGTTGTTTTGA | 97 |
| GCGCTCAAAACTT | |
| ATGGATAAAAAAATACAGCGTTTTTCATGTACAACTATACTAGTTGTTTTTG | 98 |
| AGCGCTCAAAATT | |
| CCCTATTTTGAGCGCTCAAAACTCTCCTGGGAGCGTTGACAACTTGCGCACC | 99 |
| CTGATCTG | |
| TTTTTGTATGTTAGTTGTGTCACTGGGTAGACCTAAATAGTGTTTTGAGCGCT | 100 |
| CAAAAAATTTAA | |
| TTAGTTCCTCGTTTTCTCTCGTTAAtccctatcagtgatagagaCTAAACtccctatcagtgataga | 101 |
| ga | |
| GTGAAACCAGTAACGTTATACGATGTCGCAGAGTATGCCGGTGTCTCTTAT | 102 |
| CAGACCGTTTCCCGCGTGGTGAACCAGGCCAGCCACGTTTCTGCGAAAACG | |
| CGGGAAAAAGTGGAAGCGGCGATGGCGGAGCTGAATTACATTCCCAACCGC | |
| GTGGCACAACAACTGGCGGGCAAACAGTCGTTGCTGATTGGCGTTGCCACC | |
| TCCAGTCTGGCCCTGCACGCGCCGTCGCAAATTGTCGCGGCGATTAAATCTC | |
| GCGCCGATCAACTGGGTGCCAGCGTGGTGGTGTCGATGGTAGAACGAAGCG | |
| GCGTCGAAGCCTGTAAAACGGCGGTGCACAATCTTCTCGCGCAACGCGTCA | |
| GTGGGCTGATCATTAACTATCCGCTGGATGACCAGGATGCCATTGCTGTGGA | |
| AGCTGCCTGCACTAATGTTCCGGCGTTATTTCTTGATGTCTCTGACCAGACA | |
| CCCATCAACAGTATTATTTTCTCCCATGAAGACGGTACGCGACTGGGCGTGG | |
| AGCATCTGGTCGCATTGGGTCACCAGCAAATCGCGCTGTTAGCGGGCCCATT | |
| AAGTTCTGTCTCGGCGCGTCTGCGTCTGGCTGGCTGGCATAAATATCTCACT | |
| CGCAATCAAATTCAGCCGATAGCGGAACGGGAAGGCGACTGGAGTGCCATG | |
| TCCGGTTTTCAACAAACCATGCAAATGCTGAATGAGGGCATCGTTCCCACTG | |
| CGATGCTGGTTGCCAACGATCAGATGGCGCTGGGCGCAATGCGCGCCATTA | |
| CCGAGTCCGGGCTGCGCGTTGGTGCGGATATCTCGGTAGTGGGATACGACG | |
| ATACCGAAGACAGCTCATGTTATATCCCGCCGTTAACCACCATCAAACAGG | |
| ATTTTCGCCTGCTGGGGCAAACCAGCGTGGACCGCTTGCTGCAACTCTCTCA | |
| GGGCCAGGCGGTGAAGGGCAATCAGCTGTTGCCCGTCTCACTGGTGAAAAG | |
| AAAAACCACCCTGGCGCCCAATACGCAAACCGCCTCTCCCCGCGCGTTGGC | |
| CGATTCATTAATGCAGCTGGCACGACAGGTTTCCCGACTGGAAAGCGGGCA | |
| GTGA | |
| GTGAAACCAGTAACGTTATACGATGTCGCAGAGTATGCCGGTGTCTCTTAT | 103 |
| CAGACCGTTTCCCGCGTGGTGAACCAGGCCAGCCACGTTTCTGCGAAAACG | |
| CGGGAAAAAGTGGAAGCGGCGATCAAAGAGCTGGGCTACGTGCCGAACCG | |
| CGCAGCCCGCACCCTGGTCACCCGACGTACCGACACCGTAGCCCTGGTGGT | |
| GTCGGAAAACAACCAGAAGCTCTTCGCCGAACCCTTCTATGCCGGGATCGT | |
| GCTCGGCGTGGGGGTTGCTCTGTCCGAACGGGGATTCCAGTTCGTCCTGGCC | |
| ACGGGCCGCTCCGGGATAGAGCATGAGCGGCTGGGCGGCTACCTGGCCGGA | |
| CAGCACGTCGACGGGGTCCTCCTGCTGTCGCTCCACCGCGACGACCCGCTGC | |
| CGCAGATGCTGGACGAGGCCGGGGTGCCGTACGTCTACGGCGGCCGTCCGC | |
| TCGGCGTCCCCGAAGAACAGGTGTCCTATGTCGATATCGACAACATCGGCG | |
| GGGGACGCCAGGCCACCCAGCGGCTGATCGAGACCGGGCACCGGCGGATC | |
| GCTACGATCGCGGGCCCGCAGGACATGGTCGCTGGTGTGGAACGCCTCCAG | |
| GGGTATCGCGAAGCACTGCTCGCCGCGGGGATGGAGTACGACGAGACGCTG | |
| GTGAGCTACGGTGACTTCACCTACGACAGCGGGGTGGCCGCGATGCGGGAG | |
| CTGCTGGATCGGGCCCCCGACGTGGACGCCGTGTTCGCGGCCTCCGACTTGA | |
| TGGGGCTGGCCGCGCTGCGGGTGCTGCGTGCTTCGGGACGCCGCGTGCCCG | |
| AGGATGTGGCGGTGGTCGGCTACGACGACTCGACCGTAGCCGAGCACGCCG | |
| AACCGCCGATGACCAGCGTCAACCAGCCCACCGAGCTGATGGGCCGGGAGA | |
| TGGCCCGGCTGCTCGTGGACCGGATCACCGGGGAGACCACCGAACCGGTGC | |
| GGCTGGTGCTGGAGACCCATTTGATGGTGCGGGAATCCGGGTGA | |
| GTGAAACCAGTAACGTTATACGATGTCGCAGAGTATGCCGGTGTCTCTTAT | 104 |
| CAGACCGTTTCCCGCGTGGTGAACCAGGCCAGCCACGTTTCTGCGAAAACG | |
| CGGGAAAAAGTGGAAGCGGCGATGGCGGAGCTCAATTACATTCCCAACCGC | |
| GTGGCACAACAACTGGCGGGCAAAGCGtcgCATacaATTGGCATGTTGATCAC | |
| TGCCAGTACCAATCCTTTCTATTCAGAACTGGTGCGTGGCGTTGAACGCAGC | |
| TGCTTCGAACGCGGTTATAGTCTCGTCCTTTGCAATACCGAAGGCGATGAAC | |
| AGCGGATGAATCGCAATCTGGAAACGCTGATGCAAAAACGCGTTGATGGCT | |
| TGCTGTTACTGTGCACCGAAACGCATCAACCTTCGCGTGAAATCATGCAACG | |
| TTATCCGACAGTGCCTACTGTGATGATGGACTGGGCTCCGTTCGATGGCGAC | |
| AGCGATCTTATTCAGGATAACTCGTTGCTGGGCGGAGACTTAGCAACGCAA | |
| TATCTGATCGATAAAGGTCATACCCGTATCGCCTGTATTACCGGCCCGCTGG | |
| ATAAAACTCCGGCGCGCCTGCGGTTGGAAGGTTATCGGGCGGCGATGAAAC | |
| GTGCGGGTCTCAACATTCCTGATGGCTATGAAGTCACTGGTGATTTTGAATT | |
| TAACGGCGGGTTTGACGCTATGCGCCAACTGCTATCACATCCGCTGCGTCCT | |
| CAGGCCGTCTTTACCGGAAATGACGCTATGGCTGTTGGCGTTTACCAGGCGT | |
| TATATCAGGCAGAGTTACAGGTTCCGCAGGATATCGCGGTGATTGGCTATG | |
| ACGATATCGAACTGGCAAGCTTTATGACGCCACCATTAACCACTATCCACCA | |
| ACCGAAAGATGAACTGGGGGAGCTGGCGATTGATGTACTCATCCATCGGAT | |
| AACCCAGCCGACCCTTCAGCAACAACGATTACAACTTACTCCGATTCTGATG | |
| GAACGCGGTTCGGCTTAG | |
| GTGAAACCAGTAACGTTATACGATGTCGCAGAGTATGCCGGTGTCTCTTAT | 105 |
| CAGACCGTTTCCCGCGTGGTGAACCAGGCCAGCCACGTTTCTGCGAAAACG | |
| CGGGAAAAAGTGGAAGCGGCGATGGAGTCTCTTAGCTATCACCCGAACGCC | |
| AACGCCCGTGCGCTGGCGCAGCAGACCACTGAAACGGTCGGTCTGGTCGTT | |
| GGTGATGTTTCCGATCCGTTTTTCGGTGCAATGGTGAAAGCGGTCGAACAGG | |
| TGGCTTATCACACCGGTAATTTTTTATTGATTGGCAACGGTTACCACAACGA | |
| ACAAAAAGAGCGTCAGGCCATTGAGCAACTGATCCGCCATCGCTGTGCTGC | |
| GTTGGTCGTCCATGCCAAAATGATCCCGGATGCTGATTTAGCCTCATTAATG | |
| AAACAAATGCCCGGTATGGTGCTGATCAACCGTATCCTGCCTGGCTTTGAAA | |
| ACCGTTGTATTGCTCTGGACGATCGTTACGGTGCCTGGCTGGCAACGCGTCA | |
| TTTAATTCAGCAAGGTCATACCCGCATTGGTTATCTGTGCTCTAACCACTCT | |
| ATTTCTGACGCCGAAGATCGTCTGCAAGGGTATTACGATGCCCTTGCTGAAA | |
| GTGGTATTGCGGCCAATGACCGGCTGGTGACATTTGGCGAACCAGACGAAA | |
| GCGGCGGCGAACAGGCAATGACCGAGCTTTTGGGACGAGGAAGAAATTTCA | |
| CTGCGGTAGCCTGTTATAACGATTCAATGGCGGCGGGTGCGATGGGCGTTCT | |
| CAATGATAATGGTATTGATGTACCGGGTGAGATTTCGTTAATTGGCTTTGAT | |
| GATGTGCTGGTGTCACGCTATGTGCGTCCGCGCCTGACCACCGTGCGTTACC | |
| CAATCGTGACGATGGCGACCCAGGCTGCCGAACTGGCTTTGGCGCTGGCGG | |
| ATAATCGCCCTCTCCCGGAAATCACTAATGTCTTTAGTCCGACGCTGGTACG | |
| TCGTCATTCAGTGTCAACTCCGTCGCTGGAGGCAAGTCATCATGCAACCAGC | |
| GACTAA | |
| GTGAAACCAGTAACGTTATACGATGTCGCAGAGTATGCCGGTGTCTCTTAT | 106 |
| CAGACCGTTTCCCGCGTGGTGAACCAGGCCAGCCACGTTTCTGCGAAAACG | |
| CGGGAAAAAGTGGAAGCGGCGATGGCGGAGCTCAATTACATTCCCAACCGC | |
| GTGGCACAACAACTGGCGGGCAAAAGTAGCCGTTCTATTGGTCTTGTGATC | |
| CCCGATCTGGAGAACACCAGCTATACCCGCATCGCTAACTATCTTGAACGCC | |
| AGGCGCGGCAACGGGGTTATCAACTGCTGATTGCCTGCTCAGAAGATCAGC | |
| CAGACAACGAAATGCGGTGCATTGAGCACCTTTTACAGCGTCAGGTTGATG | |
| CCATTATTGTTTCGACGTCGTTGCCTCCTGAGCATCCTTTTTATCAACGCTGG | |
| GCTAACGACCCGTTCCCGATTGTCGCGCTGGACCGCGCCCTCGATCGTGAAC | |
| ACTTCACCAGCGTGGTTGGTGCCGATCAGGATGATGCCGAAATGCTGGCGG | |
| AAGAGTTACGTAAGTTTCCCGCCGAGACGGTGCTTTATCTTGGTGCGCTACC | |
| GGAGCTTTCTGTCAGCTTCCTGCGTGAACAAGGTTTCCGTACTGCCTGGAAA | |
| GATGATCCGCGCGAAGTGCATTTCCTGTATGCCAACAGCTATGAGCGGGAG | |
| GCGGCTGCCCAGTTATTCGAAAAATGGCTGGAAACGCATCCGATGCCGCAG | |
| GCGCTGTTCACAACGTCGTTTGCGTTGTTGCAAGGAGTGATGGATGTCACGC | |
| TGCGTCGCGACGGCAAACTGCCTTCTGACCTGGCAATTGCCACCTTTGGCGA | |
| TAACGAACTGCTCGACTTCTTACAGTGTCCGGTGCTGGCAGTGGCTCAACGT | |
| CACCGCGATGTCGCAGAGCGTGTGCTGGAGATTGTCCTGGCAAGCCTGGAC | |
| GAACCGCGTAAGCCAAAACCTGGTTTAACGCGCATTAAACGTAATCTCTAT | |
| CGCCGCGGCGTGCTCAGCCGTAGCTAA | |
| GTGAAACCAGTAACGTTATACGATGTCGCAGAGTATGCCGGTGTCTCTTAT | |
| CAGACCGTTTCCCGCGTGGTGAACCAGGCCAGCCACGTTTCTGCGAAAACG | 107 |
| CGGGAAAAAGTGGAAGCGGCGATGGCGGAGCTCAATTACATTCCCAACCGC | |
| GTGGCACAACAACTGGCGGGCAAAGCGTCGCATACCATTGGCATGTTGATC | |
| ACTGCCAGTACCAATCCTTTCTATTCAGAACTGGTGCGTGGCGTTGAACGCA | |
| GCTGCTTCGAACGCGGTTATAGTCTCGATCTTTGCAATACCGAAGGCGATGA | |
| ACAGCGGATGAATCGCAATCTGGAAACGCTGATGCAAAAACGCGTTGATGG | |
| CTTGCTGTTACTGTGCACCGAAACGCATCAACCTTCGCGTGAAATCATGCAA | |
| CGTTATCCGACAGTGCCTACTGTGATGATGGACTGGGCTCCGTTCGATGGCG | |
| ACAGCGATCTTATTCAGGATAACTCGTTGCTGGGCGGAGACTTAGCAACGC | |
| AATATCTGATCGATAAAGGTCATACCCGTATCGCCTGTATTACCGGCCCGCT | |
| GGATAAAACTCCGGCGCGCCTGCGGTTGGAAGGTTATCGGGCGGCGATGAA | |
| ACGTGCGGGTCTCAACATTCCTGATGGCTATGAAGTCACTGGTGATTTTGAA | |
| TTTAACGGCGGGTTTGACGCTATGCGCCAACTGCTATCACATCCGCTGCGTC | |
| CTCAGGCCGTCTTTACCGGAAATGACGCTATGGCTGTTGGCGTTTACCAGGC | |
| GTTATATCAGGCAGAGTTACAGGTTCCGCAGGATATCGCGGTGATTGGCTAT | |
| GACGATATCGAACTGGCAAGCTTTATGACGCCACCATTAACCACTATCCACC | |
| AACCGAAAGATGAACTGGGGGAGCTGGCGATTGATGTACTCATCCATCGGA | |
| TAACCCAGCCGACCCTTCAGCAACAACGATTACAACTTACTCCGATTCTGAT | |
| GGAACGCGGTTCGGCTTAG | |
| GTGAAACCAGTAACGTTATACGATGTCGCAGAGTATGCCGGTGTCTCTTAT | 108 |
| CAGACCGTTTCCCGCGTGGTGAACCAGGCCAGCCACGTTTCTGCGAAAACG | |
| CGGGAAAAAGTGGAAGCGGCGATGGCGGAGCTCAATTACATTCCCAACCGC | |
| GTGGCACAACAACTGGCGGGCAAAAGTTCGCGTTCTATTGGCCTTGTGATCC | |
| CCGATCTGGAGAACACCAGCTATTCCCGCTTCGCTAACTATCTCGAACGCCA | |
| GGCGCGGCAACGGGGTTATCAACTGAAAATTGCCTGCTCAGAAGATCAGCC | |
| AGACAACGAAATGCGGTGCATTGAGCACCTTTTACAGCGTCAGGTTGATGC | |
| CATTATTGTTTCGACGTCGTTGCCTCCTGAGCATCCTTTTTATCAACGCTGGG | |
| CTAACGACCCGTTCCCGATTGTCGCGCTGGACCGCGCCCTCGATCGTGAACA | |
| CTTCACCAGCGTGGTTGGTGCCGATCAGGATGATGCCGAAATGCTGGCGGA | |
| AGAGTTACGTAAGTTTCCCGCCGAGACGGTGCTTTATCTTGGTGCGCTACCG | |
| GAGCTTTCTGTCAGCTTCCTGCGTGAACAAGGTTTCCGTACTGCCTGGAAAG | |
| ATGATCCGCGCGAAGTGCATTTCCTGTATGCCAACAGCTATGAGCGGGAGG | |
| CGGCTGCCCAGTTATTCGAAAAATGGCTGGAAACGCATCCGATGCCGCAGG | |
| CGCTGTTCACAACGTCGTTTGCGTTGTTGCAAGGAGTGATGGATGTCACGCT | |
| GCGTCGCGACGGCAAACTGCCTTCTGACCTGGCAATTGCCACCTTTGGCCAG | |
| AACGAACTGCTCGACTTCTTACAGTGTCCGGTGCTGGCAGTGGCTCAACGTC | |
| ACCGCGATGTCGCAGAGCGTGTGCTGGAGATTGTCCTGGCAAGCCTGGACG | |
| AACCGCGTAAGCCAAAACCTGGTTTAACGCGCATTAAACGTAATCTCTATC | |
| GCCGCGGCGTGCTCAGCCGTAGCTAA | |
| GTGAAACCAGTAACGTTATACGATGTCGCAGAGTATGCCGGTGTCTCTTAT | 109 |
| CAGACCGTTTCCCGCGTGGTGAACCAGGCCAGCCACGTTTCTGCGAAAACG | |
| CGGGAAAAAGTGGAAGCGGCGATGGCGGAGCTCAATTACATTCCCAACCGC | |
| GTGGCAGCCCAACTGGCGGGCAAACAGTCGGACACCATAGGCGTGGTGGTG | |
| ATGGACGTTACTGATGCGTTTTTCGGCGCGCTGGTAAAAGCGGTGGATCAG | |
| GTCGCTCAGCAGCATCAGAAATACGTGGCAATCGGCAATAGCTATCATGAA | |
| GCGGAAAAAGAGCGTCACGCCATTGAGGTGTTAATTCGCCAGCGTTGTAAT | |
| GCGTTGATTGTTCACTCAAAAGCATTGAGTGACGATGAACTGGCGCAATTTA | |
| TGGATAACATTCCCGGTATGGTGTTAATCAACCGCGTTGTGCCGGGGTACGC | |
| CCATCGTTGCGTTTGTCTGGATAATCTCAGCGGTGCCCGAATGGCGACGCGC | |
| ATGTTGCTGAATAACGGTCATCAACGTATTGGTTATCTTTCTTCCAGCCACG | |
| GCATTGAAGATGACGCCATGCGTAAAGCAGGCTGGATGAGTGCGTTGAAAG | |
| AGCAGGATATTATTCCGCCGGAAAGCTGGATTGGCGCTGGTACGCCGGACA | |
| TGCCGGGCGGTGAGGCGGCGTTGGTTAAACTGCTGGGGCGCAATCTACAAC | |
| TTACCGCTGTATTTGCTTATAACGACAATATGGCCGCTGGCGCACTGACAGC | |
| ATTAAAAGATAATGGCATTGCGATTCCGTTACATCTCTCAATCATCGGGTTC | |
| GATGATATTCCCATCGCCCGTTACACCGACCCGCAATTAACGACCGTGCGTT | |
| ATCCCATTGCTTCAATGGCGAAATTAGCCACCGAACTGGCCTTGCAGGGGG | |
| CAGCAGGCAATATTGATCCTCGTGCCAGCCACTGTTTTATGCCGACGTTAGT | |
| GCGTCGCCATTCTGTCGCAACACGCCAGAATGCGGCGGCGATCACTAACTC | |
| AACAAATCAGGCGATGTAA | |
| GTGAAACCAGTAACGTTATACGATGTCGCAGAGTATGCCGGTGTCTCTTAT | 110 |
| CAGACCGTTTCCCGCGTGGTGAACCAGGCCAGCCACGTTTCTGCGAAAACG | |
| CGGGAAAAAGTGGAAGCGGCGATGGCGGAGCTCCACTACATTCCTAGCGCG | |
| GTGGCGCGTAGCCTGGCGGGCAAACAGTCGAAGTCTATCGGTTTGCTGGCG | |
| ACCAGCAGCGAAGCGGCCTATTTTGCCGAGATCATTGAAGCAGTTGAAAAA | |
| AATTGCTTCCAGAAAGGTTACACCCTGATTCTGGGCAATGCGTGGAACAAT | |
| CTTGAGAAACAGCGGGCTTATCTGTCGATGATGGCGCAAAAACGCGTCGAT | |
| GGTCTGCTGGTGATGTGTTCTGAGTACCCAGAGCCGTTGCTGGCGATGCTGG | |
| AAGAGTATCGCCATATCCCAATGGTGGTCATGGACTGGGGTGAAGCAAAAG | |
| CTGACTTCACCGATGCGGTCATTGATAACGCGTTCGAAGGCGGCTACATGG | |
| CCGGGCGTTATCTGATTGAACGCGGTCACCGCGAAATCGGCGTCATCCCCG | |
| GCCCGCTGGAACGTAACACCGGCGCAGGCCGCCTTGCCGGTTTTATGAAGG | |
| CGATGGAAGAAGCGATGATCAAGGTGCCGGAAAGCTGGATTGTGCAGGGT | |
| GACTTTGAACCTGAATCCGGTTATCGCGCCATGCAGCAAATCCTGTCGCAGC | |
| CGCATCGCCCTACTGCCGTCTTCTGTGGTGGCGATATCATGGCAATGGGCGC | |
| ACTTTGTGCTGCTGATGAAATGGGCCTGCGCGTCCCGCAGGATGTTTCGCTG | |
| ATCGGTTATGATAACGTGCGCAACGCGCGCTATTTTACGCCGGCGCTGACCA | |
| CGATCCATCAGCCAAAAGATTCGCTGGGTGAAACAGCGTTCAACATGCTGT | |
| TGGATCGTATCGTCAACAAACGTGAAGAACCGCAGTCTATTGAAGTGCATC | |
| CGCGCTTGATTGAACGCCGCTCCGTGGCTGACGGCCCGTTCCGCGACTATCG | |
| TCGTTAA | |
| ATGTCCAGATTAGATAAAAGTAAAGTGATTAACAGCGCATTAGAGCTGCTT | |
| AATGAGGTCGGAATCGAAGGTTTAACAACCCGTAAACTCGCCCAGAAGCTA | 111 |
| GGTGTAGAGCAGCCTACATTGTATTGGCATGTAAAAAATAAGCGGGCTTTG | |
| CTCGACGCCTTAGCCATTGAGATGTTAGATAGGCACCATACTCACTTTTGCC | |
| CTTTAGAAGGGGAAAGCTGGCAAGATTTTTTACGTAATAACGCTAAAAGTT | |
| TTAGATGTGCTTTACTAAGTCATCGCGATGGAGCAAAAGTACATTTAGGTAC | |
| ACGGCCTACAGAAAAACAGTATGAAACTCTCGAAAATCAATTAGCCTTTTT | |
| ATGCCAACAAGGTTTTTCACTAGAGAATGCATTATATGCACTCAGCGCTGTG | |
| GGGCATTTTACTTTAGGTTGCGTATTGGAAGATCAAGAGCATCAAGTCGCTA | |
| AAGAAGAAAGGGAAACACCTACTACTGATAGTATGCCGCCATTATTACGAC | |
| AAGCTATCGAATTATTTGATCACCAAGGTGCAGAGCCAGCCTTCTTATTCGG | |
| CCTTGAATTGATCATATGCGGATTAGAAAAACAACTTAAATGTGAAAGTGG | |
| GTCCTAA | |

What is claimed is:

1. A synthetic memory system, comprising:
a recombinase;
a modified transcription factor; and
a first nucleic acid comprising a first attachment site, a second attachment site, and a target gene between said first attachment site and said second attachment site;
wherein at least one of the first attachment site and the second attachment site comprises a modified DNA operator to which the modified transcription factor can reversibly bind; and
wherein, when the modified transcription factor is bound to the modified DNA operator, the recombinase is blocked from binding to the attachment site comprising said modified DNA operator.

2. The synthetic memory system of claim 1, wherein the recombinase is a serine integrase.

3. The synthetic memory system of claim 1, wherein the modified transcription factor responds to addition or removal of an input by modifying binding of said transcription factor.

4. The synthetic memory system of claim 3, wherein adding the input causes the modified transcription factor to reversibly bind to the modified DNA operator and removing the input causes the modified transcription factor to release from the modified DNA operator.

5. The synthetic memory system of claim 3, wherein adding the input causes the modified transcription factor to release from the modified DNA operator and removing the input causes the modified transcription factor to reversibly bind to the modified DNA operator.

6. The synthetic memory system of claim 3, wherein the input is a small molecule.

7. The synthetic memory system of claim 1, wherein each of the first attachment site and the second attachment site comprises a conserved region and two half-sites on either side of said conserved region.

8. The synthetic memory system of claim 7, wherein the conserved region is from 2 nucleotides to 10 nucleotides in length; and
wherein each half-site is independently from 20 nucleotides to 40 nucleotides in length.

9. The synthetic memory system of claim 7, wherein the modified DNA operator is upstream or downstream of the conserved region; and
wherein the modified DNA operator is spaced from the conserved region by up to 10 nucleotides.

10. The synthetic memory system of claim 1, wherein the modified DNA operator is from 10 nucleotides to 20 nucleotides in length.

11. The synthetic memory system of claim 1, wherein the first attachment site is an attB site, and wherein the second attachment site is an attP site.

12. The synthetic memory system of claim 1, wherein the second attachment site comprises the modified DNA operator.

13. The synthetic memory system of claim 1, wherein each of the first attachment site and the second attachment site independently comprise about 80% identity or more to any one of SEQ ID NOs: 54-60, 65-69, 82-90, or 92-100.

14. The synthetic memory system of claim 1, wherein a first modified transcription factor and a second modified transcription factor can simultaneously reversibly bind to the modified DNA operator; and
wherein, when at least one of the first modified transcription factor and the second modified transcription factor is bound to the modified DNA operator, the recombinase is blocked from binding to the attachment site comprising said modified DNA operator.

15. The synthetic memory system of claim 1, further comprising a second nucleic acid encoding the recombinase and a third nucleic acid encoding the modified transcription factor.

16. A method of executing a memory operation, the method comprising:
a) providing a synthetic memory system comprising:
a recombinase;
a modified transcription factor; and
a first nucleic acid comprising a first attachment site, a second attachment site, and a target gene between said first attachment site and said second attachment site;
wherein at least one of the first attachment site and the second attachment site comprises a modified DNA operator to which the modified transcription factor can reversibly bind; and
wherein, when the modified transcription factor is bound to the modified DNA operator, the recombinase is blocked from binding to the attachment site comprising said modified DNA operator; and
b) adding or removing an input;
wherein the modified transcription factor responds to the addition or removal of said input by modifying binding of said transcription factor and triggering the memory operation.

17. The method of claim 16, wherein adding the input causes the modified transcription factor to reversibly bind to the modified DNA operator and removing the input causes the modified transcription factor to release from the modified DNA operator.

18. The method of claim 16, wherein adding the input causes the modified transcription factor to release from the modified DNA operator and removing the input causes the modified transcription factor to reversibly bind to the modified DNA operator.

19. The method of claim 16, wherein the memory operation is a loss-of-function of the target gene.

20. The method of claim 16, wherein the memory operation is a gain-of-function of the target gene.
